(19) United States
(12) Patent Application Publication
(10) Pub. No.: US 2025/0195487 A1
(43) Pub. Date: Jun. 19, 2025

(54) FORMULATIONS OF A CSF-1R INHIBITOR

(71) Applicant: Deciphera Pharmaceuticals, LLC, Waltham, MA (US)

(72) Inventors: Ehab Hamed, Waltham, MA (US); David P. Pleynet, Waltham, MA (US)

(21) Appl. No.: 18/980,426

(22) Filed: Dec. 13, 2024

Related U.S. Application Data
(60) Provisional application No. 63/610,751, filed on Dec. 15, 2023.

(51) Int. Cl. A61K 31/444; A61K 9/48

(52) U.S. Cl. CPC A61K 31/444; A61K 9/4833; A61K 9/4858; A61K 9/4866

(57) ABSTRACT

Described herein, in part, are pharmaceutically acceptable formulations comprising a compound represented by Formula (I) and methods of preparing and using the formulations:

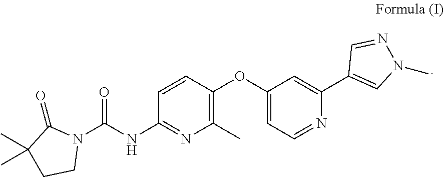

### CROSS-REFERENCE

This application claims priority to U.S. Provisional Application No. 63/610,751 filed Dec. 15, 2023, the contents of which are incorporated herein by reference in their entireties.

The present disclosure relates to pharmaceutically acceptable formulations comprising the compound represented by Formula (I), oral dosage forms comprising the compound represented by Formula (I), such as pharmaceutically acceptable capsules comprising the compound represented by Formula (I), and methods for their preparation and their use.

### BACKGROUND

Colony-stimulating factor 1 receptor (CSF-1R) and its ligand, colony stimulating factor 1 (CSF-1) together form a lineage dependency for normal macrophage development and differentiation from monocytes. As such, tumor-associated macrophages (TAMs) are dependent on CSF-1R (also known as FMS) kinase activity for proliferation, and maintenance of their differentiated state and immunosuppressive phenotype. The role of TAMs in promoting an invasive and immunosuppressive tumor microenvironment is well established. TAMs mediate tumor growth, angiogenesis, invasiveness, metastasis, and immunosuppression through the secretion of and response to a variety of cytokines or other soluble factors. TAMs are educated by tumors to enable escape from immune surveillance by dampening a cytotoxic T cell immune response, thereby shielding the tumor from T cell eradication. For example, TAMs express PD-L1, a known immunosuppressive checkpoint that induces T cell anergy.

Several inhibitors targeting CSF-1R have advanced into the clinic as direct antitumor therapies and potential immunotherapies. Many of these drugs also inhibit the closely related Type III receptor tyrosine kinases KIT, PDGFRα/β and FLT3, which may limit their utility due to off-target toxicity. Antibodies targeting CSF-1R are much more specific yet result in >10,000-fold increases in plasma levels of CSF-1, the ligand for CSF-1R, due to blockade of CSF-1 clearance, among other drawbacks.

Tenosynovial giant cell tumor (TGCT) is a proliferative and inflammatory disease that includes entities formerly known as pigmented villonodular synovitis (PVNS), and giant cell tumor of the tendon sheath (GCTTS), intraarticular or extraarticular. It is a rare neoplasm of the joint or tendon sheath, with destructive proliferation of synovial like mononuclear cells, admixed with multinucleate giant cells, foam cells, siderophages and inflammatory cells. There are two types of TGCT: the local or nodular form (where the tumor involves the tendons that support the joint, or in one area of the joint) and the diffuse form (where the entire lining of the joint is involved). Treatment is surgical excision of the tumor. However, it is often difficult to perform a marginal excision for the diffuse form of TGCT resulting in a high recurrence rate. It can be characterized by overexpression of CSF-1.

Thus, there is a need for selective small-molecule CSF-1R inhibitors and formulations thereof, for example oral dosage formulations, that are useful in the treatment of disorders associated with the proliferation of TAMs including solid tumors of various cancers and treatment of mesenchymal tumors including TGCT and diffuse-type tenosynovial giant cell tumor (DTGCT).

Content uniformity is essential to maintain consistency within a production batch or between production batches. It is an important factor regarding the safety and efficacy of a drug. Content uniformity ensures that a patient receives a reliable dose of and exposure to the active substance, thereby enabling the patient to have a safe and efficacious use of the medicine. In view of these considerations, there is a need for formulations providing content uniformity.

### SUMMARY

Described herein, in part, are pharmaceutically acceptable formulations comprising a compound represented by Formula (I):

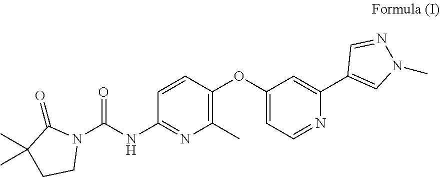

For example, in some embodiments, provided herein is a pharmaceutically acceptable unit formulation comprising: (a) about 1% by weight to about 70% by weight, based on the weight of the pharmaceutically acceptable unit formulation, of a compound of Formula (I), wherein the compound is present in the pharmaceutically acceptable unit formulation as a free base, or a pharmaceutically acceptable salt of the compound, and wherein the free base or pharmaceutically acceptable salt is in anhydrous or hydrate form, and one or more pharmaceutically acceptable excipients, wherein the one or more pharmaceutically acceptable excipients comprise: (b) about 20% by weight to about 97% by weight of one or more fillers based on the weight of the pharmaceutically acceptable unit formulation; (c) about 1% by weight to about 20% by weight of one or more disintegrants based on the weight of the pharmaceutically acceptable unit formulation; and (d) about 0.1% by weight to about 10% by weight of one or more lubricants based on the weight of the pharmaceutically acceptable unit formulation.

Also provided herein, in some embodiments, are oral dosage forms comprising a compound represented by Formula (I), and one or more pharmaceutically acceptable excipients, wherein the compound is present in the oral dosage forms as a free base, or a pharmaceutically acceptable salt of the compound, and wherein the free base or pharmaceutically acceptable salt is in anhydrous or hydrate form.

For example, in some embodiments, provided herein is an oral dosage form comprising: (a) about 1% by weight to about 70% by weight, based on the total weight of the oral dosage form, of a compound of Formula (I), wherein the compound is present in the oral dosage form as a free base, or a pharmaceutically acceptable salt of the compound, and wherein the free base or pharmaceutically acceptable salt is in anhydrous or hydrate form, and one or more pharmaceutically acceptable excipients, wherein the one or more pharmaceutically acceptable excipients comprise: (b) about 20% by weight to about 97% by weight of one or more fillers based on the total weight of the oral dosage form; (c) about 1% by weight to about 20% by weight of one or more disintegrants based on the total weight of the oral dosage form; and (d) about 0.1% by weight to about 10% by weight of one or more lubricants based on the total weight of the oral dosage form.

In some embodiments, provided herein are pharmaceutically acceptable capsules comprising a compound of Formula (I), and one or more pharmaceutically acceptable excipients, wherein the compound is present in the pharmaceutically acceptable capsules as a free base, or a pharmaceutically acceptable salt of the compound, and wherein the free base or pharmaceutically acceptable salt is in anhydrous or hydrate form.

For example, in some embodiments, provided herein is a pharmaceutically acceptable capsule comprising: (a) about 1% by weight to about 70% by weight, based on the total weight of the pharmaceutically acceptable capsule, of a compound of Formula (I), wherein the compound is present in the pharmaceutically acceptable capsule as a free base, a pharmaceutically acceptable salt of the compound, and wherein the free base or pharmaceutically acceptable salt is in anhydrous or hydrate form, and one or more pharmaceutically acceptable excipients, wherein the pharmaceutically acceptable excipients comprise: (b) about 20% by weight to about 97% by weight of one or more fillers based on the total weight of the pharmaceutically acceptable capsule; (c) about 1% by weight to about 20% by weight of one or more disintegrants based on the total weight of the pharmaceutically acceptable capsule; and (d) about 0.1% by weight to about 10% by weight of one or more lubricants based on the total weight of the pharmaceutically acceptable capsule.

Provided herein, in part, are methods of treating diseases and conditions including, but not limited to, a tenosynovial giant cell tumor (TGCT) including diffuse-type tenosynovial giant cell tumor (DTGCT) and localized tenosynovial giant cell tumor. Provided herein, in part, are methods of treating diseases and conditions including, but not limited to, graft versus host disease (GVHD) including chronic graft versus host disease (cGVHD) and acute graft versus host disease (aGVHD). Provided herein, in part, are methods of treating diseases and conditions including, but not limited to, a neurodegenerative diseases or conditions including Parkinson's disease (PD), Alzheimer's Disease (AD), frontotemporal dementia (FTD), amyotrophic lateral sclerosis (ALS), mild cognitive impairment, and Huntington's Disease (HD). Provided herein, in part, are methods of treating diseases and conditions including, but not limited to, solid tumors, acute myeloid leukemia (AML), relapsed/refractory acute myeloid leukemia (AML), relapsed acute myeloid leukemia (AML), refractory acute myeloid leukemia (AML), myelodysplastic syndrome, acute lymphocytic leukemia (ALL), chronic lymphocytic leukemia (CLL), pancreatic ductal adenocarcinoma (PDAC), ovarian cancer, non-small cell lung cancer (NSCLC), prostate cancer, osteosarcoma, breast cancer, colon cancer, and glioblastoma, wherein solid tumors include, but are not limited to, ovarian cancer, pancreatic cancer, prostate cancer, lung cancer, breast cancer, kidney cancer, liver cancer, cervical cancer, bone metastatic cancer, papillary thyroid cancer, non-small cell lung cancer, colon cancer, and gastrointestinal stromal tumors. Provided herein, in part, are methods of treating diseases and conditions including, but not limited to, tumors known to have expression of colony-stimulating factor 1 receptor (CSF-1R) or its ligands, colony stimulating factor-1 (CSF-1), or interleukin (IL)-34 (IL-34). Provided herein, in part, are methods of treating diseases and conditions including, but not limited to, metabolic diseases, rheumatoid arthritis, osteoarthritis, multiple sclerosis, autoimmune nephritis, lupus, Crohn's disease, asthma, chronic obstructive pulmonary diseases, osteoporosis, hypereosinophilic syndromes, mastocytosis, and histiocytosis.

### BRIEF DESCRIPTION OF THE DRAWINGS

FIG. 1 shows an X-ray powder diffraction pattern of the hydrochloride crystalline form I of the compound of the formula (I) of the present invention. The abscissa represents the 2θ value (degrees), and the ordinate represents the peak intensity.

FIG. 2 shows an X-ray powder diffraction pattern of the hydrochloride crystalline form II of the compound of the formula (I) of the present invention. The abscissa represents the 2θ value (degrees), and the ordinate represents the peak intensity.

FIG. 3 shows an X-ray powder diffraction pattern of the hydrochloride crystalline form III of the compound of the formula (I) of the present invention. The abscissa represents the 2θ value (degrees), and the ordinate represents the peak intensity.

FIG. 4 shows an X-ray powder diffraction pattern of the sulfate crystalline form I of the compound of the formula (I) of the present invention. The abscissa represents the 2θ value (degrees), and the ordinate represents the peak intensity.

FIG. 5 shows an X-ray powder diffraction pattern of the phosphate crystalline form I of the compound of the formula (I) of the present invention. The abscissa represents the 2θ value (degrees), and the ordinate represents the peak intensity.

FIG. 6 shows an X-ray powder diffraction pattern of the phosphate crystalline form II of the compound of the formula (I) of the present invention. The abscissa represents the 2θ value (degrees), and the ordinate represents the peak intensity.

FIG. 7 shows an X-ray powder diffraction pattern of the phosphate crystalline form III of the compound of the formula (I) of the present invention. The abscissa represents the 2θ value (degrees), and the ordinate represents the peak intensity.

FIG. 8 shows an X-ray powder diffraction pattern of the mesylate crystalline form I of the compound of the formula (I) of the present invention. The abscissa represents the 2θ value (degrees), and the ordinate represents the peak intensity.

FIG. 9 shows an X-ray powder diffraction pattern of the mesylate crystalline form II of the compound of the formula (I) of the present invention. The abscissa represents the 2θ value (degrees), and the ordinate represents the peak intensity.

FIG. 10 shows an X-ray powder diffraction pattern of the citrate crystalline form I of the compound of the formula (I) of the present invention. The abscissa represents the 2θ value (degrees), and the ordinate represents the peak intensity.

FIG. 11 shows an X-ray powder diffraction pattern of the malate crystalline form I of the compound of the formula (I) of the present invention. The abscissa represents the 2θ value (degrees), and the ordinate represents the peak intensity.

FIG. 12 shows an X-ray powder diffraction pattern of the tartrate crystalline form I of the compound of the formula (I) of the present invention. The abscissa represents the 2θ value (degrees), and the ordinate represents the peak intensity.

FIG. 13 shows an X-ray powder diffraction pattern of the fumarate crystalline form I of the compound of the formula (I) of the present invention. The abscissa represents the 2θ value (degrees), and the ordinate represents the peak intensity.

FIG. 14 shows a DSC/TGA graph of the hydrochloride crystalline form I of the compound of the formula (I) of the present invention. The abscissa represents temperature (° C.), and the ordinate represents heat flow (w/g)/weight (%).

FIG. 15 shows a DVS graph of the hydrochloride crystalline form I of the compound of the formula (I) of the present invention. The abscissa represents the relative humidity (%), and the ordinate represents the weight change (%).

FIG. 16 shows a DSC/TGA graph of the phosphate crystalline form I of the compound of the formula (I) of the present invention. The abscissa represents temperature (° C.), and the ordinate represents heat flow (w/g)/weight (%).

FIG. 17 shows a DVS graph of the phosphate crystalline form I of the compound of formula (I) of the present invention. The abscissa represents the relative humidity (%), and the ordinate represents the weight change (%).

FIG. 18 shows a DSC/TGA graph of the mesylate crystalline form II of the compound of the formula (I) of the present invention. The abscissa represents temperature (° C.), and the ordinate represents heat flow (w/g)/weight (%).

FIG. 19 shows a DVS graph of the mesylate crystalline form II of the compound of the formula (I) of the present invention. The abscissa represents the relative humidity (%), and the ordinate represents the weight change (%).

FIG. 20 shows a DSC/TGA graph of the tartrate crystalline form I of the compound of the formula (I) of the present invention. The abscissa represents temperature (° C.), and the ordinate represents heat flow (w/g)/weight (%).

FIG. 21 shows a DVS graph of the tartrate crystalline form I of the compound of the formula (I) of the present invention. The abscissa represents the relative humidity (%), and the ordinate represents the weight change (%).

FIG. 22 shows a superposed graph of a hydrochloride single-crystal simulated diagram of the compound of the formula (I) of the present invention and an X-ray powder diffraction pattern of the hydrochloride crystalline form I, with the powder-crystal diffraction peak pattern (upper) and the single-crystal simulated diagram (lower). The abscissa represents the 2θ value (degrees), and the ordinate represents the peak intensity.

FIG. 23 shows a single-crystal cell structure of the hydrochloride crystalline form I of the compound of the formula (I) of the present invention.

### DETAILED DESCRIPTION

The features and other details of the disclosure will now be more particularly described. Certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

Terms used in the singular will also include the plural. For example, “a” means one or more unless indicated otherwise.

All ranges recited herein include the endpoints, including those that recite a range “between” two values. The terms “substantially” and “about” are to be construed as modifying a term or value such that it is not an absolute. This includes, at very least, the degree of expected experimental variance, experimental error, technique variance, technique error and instrument variance, instrumental error for a given technique used to measure a value.

As used herein, “about” includes and describes the value or parameter per se. For example, “about x” includes and describes “x” per se. In some embodiments, the term “about” when used in association with a measurement, or used to modify a value, a unit, a constant, or a range of values, refers to variations of 10%. “About” in context of XRPD means±0.2° at 2-theta for XRPD peaks.

As used herein, the term “adding” does not limit the order, method or how the materials being added are combined, unless indicated otherwise. For instance, “adding X to Y” may also describe “adding Y to X.” Furthermore, “adding X and Y to Z” may also describe the various other combinations such as “adding X to Y and Z,” “adding X and Z to Y,” “adding Y to X and Z,” “adding Y and Z to X,” and “adding Z to X and Y.”

As used herein, the term “excipient” refers to a substance that may be beneficial to include in a formulation with an active agent. The term “excipient” includes inert substances as well as functional excipients that may result in beneficial properties of the formulation. Exemplary excipients include but are not limited to polymers, glidants, sugars, lubricants, salts, buffers, fats, fillers, disintegrating agents, binders, surfactants, high surface area substrates, flavorants, carriers, matrix materials, diluents, and so forth.

As used herein, the terms “individual,” “patient,” or “subject” are used interchangeably and include any animal, including mammals, preferably mice, rats, other rodents, rabbits, dogs, cats, swine, cattle, sheep, horses, or primates, and most preferably humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

As used herein, the terms “pharmaceutically acceptable” or “pharmacologically acceptable” includes molecular entities and formulations that do not produce an adverse, allergic or other untoward reaction when administered to an animal, or a human, as appropriate. For human administration, preparations should meet sterility, pyrogenicity, and general safety and purity standards as required by FDA standards.

As used herein, the term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The formulations may also contain other active compounds providing supplemental, additional, or enhanced therapeutic functions.

As used herein, the term “formulation” as used herein refers to a composition comprising at least one compound as disclosed herein formulated together with one or more pharmaceutically acceptable carriers, excipients or diluents.

As used herein, the term “pharmaceutically acceptable salt(s)” refers to salts of acidic or basic groups that may be present in compounds used in the formulations. Compounds included in the present formulations that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

As used herein, the term “therapeutically effective amount” means the amount of the subject compound that will elicit the biological or medical response of a tissue, system or animal, (e.g., mammal or human) that is being sought by the researcher, veterinarian, medical doctor or other clinician. A compound described herein, e.g., the compound represented by Formula (I), is administered in therapeutically effective amounts to treat a condition, e.g., TGCT, GVHD, or neurodegenerative diseases. Alternatively, a therapeutically effective amount of a compound is the quantity required to achieve a desired therapeutic and/or prophylactic effect, such as an amount which results in the prevention of or a decrease in the symptoms associated with the condition.

As used herein and unless otherwise indicated, the terms “treat,” “treating” and “treatment” refer to the alleviation of a disease or disorder and/or at least one of its attendant symptoms, and includes any effect, e.g., lessening, reducing, modulating, or eliminating, that results in the improvement of the condition, disease, disorder and the like.

As used herein and unless otherwise indicated, the terms “prevent,” “preventing” and “prevention” refer to the inhibition of a symptom of a disease or disorder or the disease itself.

As used herein, the term “active agent” means a drug, medicament, pharmaceutical, therapeutic agent, for example, the compound represented by Formula (I), as described herein.

As used herein, “TAM” refers to tumor-associated macrophage.

As used herein, “TGCT” refers to tenosynovial giant cell tumor.

As used herein, “DTGCT” refers to diffuse or diffuse-type tenosynovial giant cell tumor.

As used herein, “GCTTS” refers to giant cell tumor of the tendon sheath.

As used herein, “PVNS” refers to pigmented villonodular synovitis.

As used herein, “GVHD” refers to graft versus host disease.

As used herein, “AD” refers to Alzheimer's Disease.

As used herein, “PD” refers to Parkinson's Disease.

As used herein, “HD” refers to Huntington's Disease.

As used herein, “FTD” refers to frontotemporal dementia.

As used herein, “ALS” refers to amyotrophic lateral sclerosis.

As used herein and unless otherwise indicated, the terms “polymorph” and “polymorphic form” refer to solid crystalline forms of a compound or complex. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability).

As used herein, a polymorphic form may be described by reference to patterns, spectra, or other graphical data as “substantially” shown or depicted in a figure, or by one or more data points. It will be appreciated that patterns, spectra, and other graphical data can be shifted in their positions, relative intensities, or other values due to a number of factors known to those of skill in the art. For example, in the crystallographic and powder X-ray diffraction arts, shifts in peak positions or the relative intensities of one or more peaks of a pattern can occur because of, without limitation, the equipment used, the sample preparation protocol, preferred packing and orientations, the radiation source, operator error, method and length of data collection, or the like. However, those of ordinary skill in the art will be able to compare the figures herein with patterns, etc. generated for an unknown form of, in this case, the compound represented by Formula (I), and confirm its identity with the forms disclosed herein. The same holds true for other techniques which may be reported herein.

The occurrence of different polymorphs is possible for some compounds. A single compound may give rise to a variety of solids having distinct physical properties, such as X-ray diffraction patterns, infrared absorption spectra, and NMR spectra. This variation in solid forms may be significant and may result in differences with respect to bioavailability, stability, and other differences for formulated pharmaceutical products. Because polymorphic forms can vary in their physical properties, regulatory authorities require that efforts shall be made to identify all polymorphic forms, e.g., crystalline, amorphous, solvate, hydrate, etc., of new drug substances.

While the existence and possible numbers of polymorphic forms for a given pharmaceutical compound cannot be predicted, different polymorphs can possess different properties such as stability, solubility, melting point, or compressibility. As a result, new forms of a pharmaceutically useful compound may provide an opportunity to improve its characteristics, and ultimately its performance. Further, discovery of additional polymorphic forms, including solvate polymorphs, may help in the identification of the polymorphic content of a batch of an active pharmaceutical ingredient. For example, in some cases, different polymorphs of the same drug can exhibit very different solubility and different dissolution rates.

Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

Polymorphs of a molecule can be obtained by a number of methods known in the art.

Such methods include, but are not limited to, melt recrystallization, melt cooling, solvent recrystallization, desolvation, rapid evaporation, rapid cooling, slow cooling, vapor diffusion and sublimation. Polymorphs can be detected, identified, classified and characterized using well-known techniques such as, but not limited to, differential scanning calorimetry (DSC), thermogravimetry (TGA), X-ray powder diffractometry (XRPD), single crystal X-ray diffractometry, vibrational spectroscopy, solution calorimetry, solid-state nuclear magnetic resonance (NMR), infrared (IR) spectroscopy, Raman spectroscopy, hot stage optical microscopy, scanning electron microscopy (SEM), electron crystallography and quantitative analysis, particle size analysis (PSA), surface area analysis, solubility, and rate of dissolution.

Different solid-state forms can be characterized by scattering techniques, e.g., x-ray powder diffraction (XRPD) pattern, by spectroscopic methods, e.g., infrared absorption fingerprint, Raman absorption fingerprint, nuclear magnetic resonance (e.g., NMR, solid-state NMR) spectroscopy (e.g., ¹H, ¹³C, ¹⁹F), and by thermal techniques, e.g., differential scanning calorimetry (DSC) or differential thermal analysis (TGA). Described herein are polymorphs (solid-state forms) of the compound represented by Formula (I). These solid-state forms and their distinct crystal structures and physical properties are characterized by TGA, DSC (measurement of melting point and thermal behavior), XRPD, and NMR such as proton NMR spectrum (¹H NMR) and solid-state NMR spectrum (e.g., ¹³C ssNMR).

As used herein to refer to the spectra or data presented in graphical form (e.g., XRPD, IR, Raman and NMR spectra), and unless otherwise indicated, the term “peak” refers to a peak or other special feature that one skilled in the art would recognize as not attributable to background noise.

Generally, a diffraction angle (2θ, “2 theta”) in X-ray powder diffractometry may have a variation in the range of ±0.2°. Therefore, the aforementioned diffraction angle values should be understood as including values in the range of about ±0.2°. Accordingly, the solid-state forms described here includes not only crystals whose peak diffraction angles in powder X-ray diffractometry completely coincide with each other, but also crystals whose peak diffraction angles coincide with each other with an error of about ±0.0.2°. Therefore, in the present specification, the phrase “having a diffraction peak at a diffraction angle (2θ±0.2°) of 7.9°” means “having a diffraction peak at a diffraction angle (2θ) of 7.7° to 8.1°.” Although the intensities of peaks in the x-ray powder diffraction patterns of different batches of a compound may vary slightly, the peak locations are characteristic for a specific polymorphic form. Alternatively, the term “about” means within an acceptable standard error of the mean, when considered by one of ordinary skill in the art. The relative intensities of the XRPD peaks can vary depending on the sample preparation technique, crystal size distribution, various filters used, the sample mounting procedure, and the particular instrument employed. Moreover, instrument variation and other factors can affect the 2-theta values. Therefore, the term “substantially” in the context of XRPD is meant to encompass that peak assignments can vary by plus or minus about 0.2 degree. Moreover, new peaks may be observed, or existing peaks may disappear, depending on the type of the machine or the settings (for example, whether a Ni filter is used or not).

In general, provided herein are solid-state forms of the compound represented by Formula (I) that are substantially free of any other solid-state forms whether as individual forms or mixtures of other forms, unless indicated otherwise. For example, a anhydrous or hydrate form of a hydrochloride salt of the compound represented by Formula (I) will be substantially free of other forms of compound of formula (I) which may include the crystalline anhydrous form or other solid-state forms or mixtures thereof. As used herein, “substantially free of any solid-state forms” means that the solid-state form of the compound represented by Formula (I) contains about 20% or less, about 10% or less, about 5% or less, about 2% or less, or about 1% or less, of any other solid-state form of the compound represented by Formula (I) as measured, for example, by XRPD. In some embodiments, the solid-state form of the compound represented by Formula (I) contains less than about 20%, less than about 10%, less than about 5%, less than about 4%, less than about 3%, less than about 2%, or less than about 1% of any other solid-state forms of the compound represented by Formula (I) as measured, for example, by XRPD. Thus, a solid-state form of the compound represented by Formula (I) described herein as substantially free of any other solid-state forms would be understood to contain greater than 80% (w/w), greater than 90% (w/w), greater than 95% (w/w), greater than 98% (w/w), or greater than 99% (w/w) of the said solid-state forms of the compound represented by Formula (I). Accordingly, in some embodiments, the described solid-state forms of the compound represented by Formula (I) may contain from 1% to 20% (w/w), from 5% to 20% (w/w), or from 5% to 10% (w/w) of one or more other solid-state forms of the compound represented by Formula (I).

As used herein, “substantially free” means that the solid-state forms of the present disclosure contain 20% (w/w) or less of other polymorphs, or, alternatively, of a specified polymorph of the compound represented by Formula (I). According to some embodiments, the solid-state forms of the present disclosure contain 10% (w/w) or less, 5% (w/w) or less, 2% (w/w), 1% (w/w) or less of other polymorphs, or specified polymorphs of the compound represented by Formula (I). In other embodiments, solid-state forms of the compound represented by Formula (I) of the present disclosure contain from 1% to 20% (w/w), from 5% to 20% (w/w), or from 5% to 10% (w/w) of other solid-state forms, or of a specified polymorph of the compound represented by Formula (I).

As used herein and unless otherwise indicated, the term “substantially pure” when used to describe a polymorph of a compound means a solid form of the compound that comprises that polymorph and is substantially free of other polymorphs of the compound. For example, a anhydrous or hydrate form of a hydrochloride salt of the compound represented by Formula (I) will be substantially pure of other forms of compound of formula (I) which may include the crystalline anhydrous form or other solid-state forms or mixtures thereof. A representative substantially pure polymorph comprises greater than about 80% by weight of one polymorphic form of the compound and less than about 20% by weight of other polymorphic forms of the compound. In some embodiments, a representative substantially pure polymorph comprises greater than about 90% by weight of one polymorphic form of the compound and less than about 10% by weight of the other polymorphic forms of the compound. In some embodiments, a representative substantially pure polymorph comprises greater than about 95% by weight of one polymorphic form of the compound and less than about 5% by weight of the other polymorphic forms of the compound. In some embodiments, a representative substantially pure polymorph comprises greater than about 97% by weight of one polymorphic form of the compound and less than about 3% by weight of the other polymorphic forms of the compound. In some embodiments, a representative substantially pure polymorph comprises greater than about 98% by weight of one polymorphic form of the compound and less than about 2% by weight of the other polymorphic forms of the compound. In some embodiments, a representative substantially pure polymorph comprises greater than about 99% by weight of one polymorphic form of the compound and less than about 1% by weight of the other polymorphic forms of the compound.

The content of solid-state forms is typically measured by any suitable method appreciated by a skilled person in the art, for example XRPD, solid-state NMR, IR, Raman, or DSC.

Crystalline and partially crystalline solid forms may be prepared by a variety of methods including, but not limited to, for example, crystallization or recrystallization from a suitable solvent mixture; sublimation; growth from a melt; solid-state transformation from another phase; crystallization from a supercritical fluid; antisolvent addition; slurrying at various temperatures (e.g., at room temperature, at 10° C., at 15° C., at 40° C., at 50° C., at 70° C.); solid vapor diffusion; liquid vapor diffusion; evaporation; slow cooling, polymer induced crystallization; milling; spray freezing; spray congealing; lyophilization; and humidity induced crystallization. Techniques for crystallization or recrystallization of crystalline and partially crystalline solid forms of a solvent mixture include, but are not limited to, for example, evaporation of the solvent; decreasing the temperature of the solvent mixture; crystal seeding of a supersaturated solvent mixture of the compound and/or salt thereof; crystal seeding a supersaturated solvent mixture of the compound and/or a salt from thereof; freeze drying the solvent mixture; temperature cycling; and adding anti-solvents (countersolvents) to the solvent mixture. As used herein, the term “anti-solvent” refers to a liquid that, when combined with a solution of the compound represented by Formula (I), reduces solubility of the compound represented by Formula (I) in the solution, causing crystallization or precipitation in some instances spontaneously, and in other instances with additional steps, such as seeding, cooling, scratching and/or concentrating. Crystals of drugs, including polymorphs, methods of preparation, and characterization of drug crystals, are discussed in Solid-State Chemistry of Drugs, S. R. Byrn, R. R. Pfeiffer, and J. G. Stowell, 2^{nd} Edition, SSCI, West Lafayette, Ind. (1999). In a crystallization technique in which solvent is employed, the solvent(s) are typically chosen based on one or more factors including, but not limited to, for example, solubility of the compound; crystallization technique utilized; and vapor pressure of the solvent. Combinations of solvents may be employed. For example, the compound may be solubilized in a first solvent to afford a solution to which antisolvent is then added to decrease the solubility of the compound represented by Formula (I) in the solution and precipitate the formation of crystals. An antisolvent is a solvent in which a compound has low solubility. In one method that can be used in preparing crystals, a compound can be suspended and/or stirred in a suitable solvent to afford a slurry, which may be heated to promote dissolution. A cooled crystallization mixture may be filtered under vacuum and the isolated solid product washed with a suitable solvent, such as, for example, cold recrystallization solvent. After being washed, the product may be dried under a nitrogen purge to afford the desired solid form. After being washed, the product may be dried under vacuum to afford the desired solid form.

As used herein, the term “based on the total weight of the pharmaceutically acceptable capsule” refers to the total weight of the compound of Formula (I), as a free base or a pharmaceutically acceptable salt thereof, or wherein the free base or a pharmaceutically acceptable salt thereof is in anhydrous or hydrate form (e.g., a crystalline hydrate or crystalline anhydrous form of the hydrochloride salt of the compound of Formula (I)), and one or more pharmaceutically acceptable excipients (e.g., one or more fillers, and optionally one or more disintegrants and/or one or more lubricants), and does not include the weight of the empty capsule shell.

“Twice a week” and “twice weekly” as used herein interchangeably.

### Formulations

The present disclosure encompasses formulations comprising a compound represented by Formula (I)

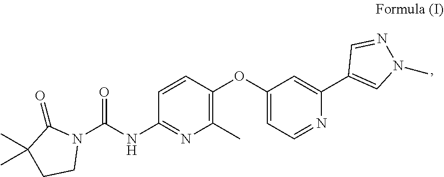

and one or more pharmaceutically acceptable excipients, wherein the compound is present in the pharmaceutically acceptable formulation as a free base, or a pharmaceutically acceptable salt of the compound.

The formulations provided herein can contain one or more fillers, which are added, for example, to increase the bulk weight of the blend resulting in a practical size for encapsulation or compression. Fillers that may be used include, but are not limited to, calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc.

A disintegrant may be present in an amount necessary to expedite dissolution (e.g., increase the rate of tablet or capsule disintegration). The term “disintegrant” as used herein refers to an excipient which can oppose the physical forces of particle bonding in a tablet or capsule when the oral formulation is placed in an aqueous environment. Disintegrants include, but are not limited to, sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharide.

The formulations can also include a lubricant. The term “lubricant” as used herein is typically added to prevent the tablet or capsule materials from sticking to punches or pins, minimize friction during tablet compression or encapsulation, and to allow for removal of the compressed tablet from the die or improve flowability of blends in capsules for improved processing properties. Examples of lubricants include, but are not limited to, colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, copolymer comprising poly(ethylene oxide) and poly(propylene oxide) (such as poloxomer 188), copolymer comprising polypropylene glycol and polyethylene glycol (such as poloxomer 407), sodium lauryl sulfate, magnesium stearate, aluminum stearate, calcium stearate, sodium stearyl fumarate, stearic acid, magnesium lauryl stearate, mixtures of magnesium stearate with sodium lauryl sulfate.

Flavoring agents and flavor enhancers may also be added for the dosage form to be more palatable to the patient. Common flavoring agents and flavor enhancers for pharmaceutical products that can be included in the formulation of the present disclosure include maltol, vanillin, ethyl vanillin, menthol, citric acid, fumaric acid, ethyl maltol, and tartaric acid.

Solid and liquid formulations can also be dyed using any pharmaceutically acceptable colorant to improve their appearance and/or facilitate patient identification of the product and unit dosage level.

In liquid formulations of the present disclosure, the active ingredient and any other solid excipients may be dissolved or suspended in a liquid carrier such as water, vegetable oil, alcohol, polyethylene glycol, propylene glycol, or glycerin.

Liquid formulations can contain emulsifying agents to disperse uniformly throughout the formulation an active ingredient or other excipient that is not soluble in the liquid carrier. Emulsifying agents that can be useful in liquid formulations of the present disclosure include, for example, gelatin, egg yolk, casein, cholesterol, acacia, tragacanth, chondrus, pectin, methyl cellulose, carbomer, cetostearyl alcohol, and cetyl alcohol.

Liquid formulations of the present disclosure can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid formulation can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid formulations of the present disclosure include powders, granulates, aggregates, and compacted formulations. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the present disclosure is oral.

Diluents increase the bulk of a solid formulation and can make a pharmaceutical dosage form containing the formulation easier for the patient and caregiver to handle. Diluents for solid formulations include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid formulations that are compacted into a dosage form, such as a tablet, can include excipients whose functions include helping to bind the active ingredient and other excipients together after compression. Binders for solid formulations include acacia, alginic acid, carbomer (e.g., Carbopol), carboxymethylcellulose sodium, dextrin, ethyl cellulose, gelatin, guar gum, hydrogenated vegetable oil, hydroxyethyl cellulose, hydroxypropyl cellulose (e.g., Klucel®), hydroxypropyl methyl cellulose (e.g., Methocel®), liquid glucose, magnesium aluminum silicate, maltodextrin, methylcellulose, polymethacrylates, povidone (e.g., Kollidon®, Plasdone®), pregelatinized starch, sodium alginate, and starch.

The dissolution rate of a compacted solid formulation in the patient's stomach can be increased by the addition of a disintegrant to the formulation. Disintegrants include alginic acid, carboxymethylcellulose calcium, carboxymethylcellulose sodium (e.g., Ac-Di-Sol®, Primellose®), colloidal silicon dioxide, croscarmellose sodium, Crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid formulation and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered formulation, the formulation is subjected to pressure from a punch and dye. Some excipients and active ingredients tend to adhere to the surfaces of the punch and dye, which can cause the product to have pitting and other surface irregularities. A lubricant can be added to the formulation to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

A formulation for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting formulation can be prepared conventionally by dry blending. For example, the blended formulation of the actives and excipients can be compacted into a slug or a sheet and then comminuted into compacted granules. The compacted granules can subsequently be compressed into a tablet.

As an alternative to dry granulation, a blended formulation can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

Dosage forms include solid dosage forms like tablets, powders, capsules, suppositories, sachets, troches, and lozenges, as well as liquid syrups, suspensions, and elixirs.

The dosage form of the present disclosure can be a capsule containing the formulation, such as a powdered or granulated solid formulation of the disclosure, within either a hard or soft shell. The shell can be made from gelatin and optionally contain a plasticizer such as glycerin and sorbitol, and an opacifying agent or colorant.

In further embodiments, a pharmaceutical formulation of the compound represented by Formula (I) is formulated for administration to a mammal, such as a human. The compound represented by Formula (I) can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringe ability), fluidity, boiling point, miscibility, and purity. Suitable solvents include alcohol USP (United States Pharmacopeia), benzyl alcohol NF, benzyl benzoate USP, and Castor oil USP. Additional substances can be added to the formulation such as buffers, solubilizers, and antioxidants, among others. Ansel et al, Pharmaceutical Dosage Forms and Drug Delivery Systems, 7^{th} ed.

In some embodiments, provided herein are pharmaceutically acceptable formulations comprising a compound of Formula (I):

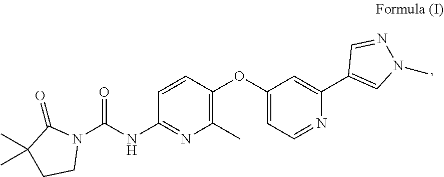

and one or more pharmaceutically acceptable excipients, wherein the compound is present in the pharmaceutically acceptable formulations as a free base, or a pharmaceutically acceptable salt of the compound, and wherein the free base or pharmaceutically acceptable salt is in anhydrous or hydrate form.

In some embodiments, the compound of Formula (I) is present in the pharmaceutically acceptable formulation as a hydrate form (the compound of Formula (I)·nH₂O). For example, the hydrate form is a hemihydrate form, a monohydrate form, a sesquihydrate form, a dihydrate form, a trihydrate form, a tetrahydrate form, a pentahydrate form, a hexahydrate form, a heptahydrate form, an octahydrate form, a nonahydrate form, or a decahydrate form. In some embodiments, the hydrate form is a hemihydrate form, a monohydrate form, a sesquihydrate form, a dihydrate form, a trihydrate form, or a tetrahydrate form. In some embodiments, the hydrate form is a hemihydrate form. In some embodiments, the hydrate form is a monohydrate form. In some embodiments, the hydrate form is a dihydrate form. In some embodiments, the compound of Formula (I) is present in the pharmaceutically acceptable formulation as a dihydrate form. In some embodiments, the compound of Formula (I) is present in the pharmaceutically acceptable formulation as an anhydrous form.

In some embodiments, a pharmaceutically acceptable salt of the compound of Formula (I) (e.g., hydrochloride salt) is present in the pharmaceutically acceptable formulation as an anhydrous form. In some embodiments, a pharmaceutically acceptable salt of the compound of Formula (I) (e.g., hydrochloride salt) is present in the pharmaceutically acceptable formulation as a hydrate form. For example, the hydrate form is a hemihydrate form, a monohydrate form, a sesquihydrate form, a dihydrate form, a trihydrate form, a tetrahydrate form, a pentahydrate form, a hexahydrate form, a heptahydrate form, an octahydrate form, a nonahydrate form, or a decahydrate form. In some embodiments, the hydrate form is a hemihydrate form, a monohydrate form, a sesquihydrate form, a dihydrate form, a trihydrate form, or a tetrahydrate form. In some embodiments, the hydrate form is a hemihydrate form. In some embodiments, the hydrate form is a monohydrate form. In some embodiments, the hydrate form is a dihydrate form. In some embodiments, the compound of Formula (I) is present in the pharmaceutically acceptable formulation as a dihydrate form. In some embodiments, the compound of Formula (I) is present in the pharmaceutically acceptable formulation as an anhydrous form.

In some embodiments, the one or more pharmaceutically acceptable excipients is a filler. For example, the one or more pharmaceutically acceptable excipients is lactose, such as lactose monohydrate.

In some embodiments, the pharmaceutically acceptable formulations further comprise one or more disintegrants, one or more lubricants, or a combination thereof. For example, in some embodiments, the pharmaceutically acceptable formulations further comprise one or more disintegrants. In some embodiments, the pharmaceutically acceptable formulations further comprise one or more lubricants.

In some embodiments, provided herein are pharmaceutically acceptable unit formulations comprising a compound of Formula (I):

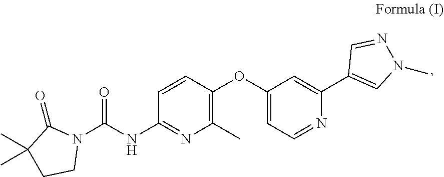

and one or more pharmaceutically acceptable excipients, wherein the compound is present in the pharmaceutically acceptable unit formulations as a free base, or a pharmaceutically acceptable salt of the compound, and wherein the free base or pharmaceutically acceptable salt is in anhydrous or hydrate form.

In some embodiments, the compound of Formula (I) is present in the pharmaceutically acceptable unit formulation as a hydrate form (the compound of Formula (I)·nH₂O). For example, the hydrate form is a hemihydrate form, a monohydrate form, a sesquihydrate form, a dihydrate form, a trihydrate form, a tetrahydrate form, a pentahydrate form, a hexahydrate form, a heptahydrate form, an octahydrate form, a nonahydrate form, or a decahydrate form. In some embodiments, the hydrate form is a hemihydrate form, a monohydrate form, a sesquihydrate form, a dihydrate form, a trihydrate form, or a tetrahydrate form. In some embodiments, the hydrate form is a hemihydrate form. In some embodiments, the hydrate form is a monohydrate form. In some embodiments, the hydrate form is a dihydrate form. In some embodiments, the compound of Formula (I) is present in the pharmaceutically acceptable unit formulation as a dihydrate form. In some embodiments, the compound of Formula (I) is present in the pharmaceutically acceptable unit formulation as an anhydrous form.

In some embodiments, the one or more pharmaceutically acceptable excipients is a filler. For example, the one or more pharmaceutically acceptable excipients is lactose, such as lactose monohydrate.

In some embodiments, the pharmaceutically acceptable unit formulations further comprise one or more disintegrants, one or more lubricants, or a combination thereof. For example, in some embodiments, the pharmaceutically acceptable unit formulations further comprise one or more disintegrants. In some embodiments, the pharmaceutically acceptable unit formulations further comprise one or more lubricants.

Also provided herein, in some embodiments, is a pharmaceutically acceptable unit formulation comprising:
(a) about 1% by weight to about 70% by weight, based on the weight of the pharmaceutically acceptable unit formulation, of a compound of Formula (I):

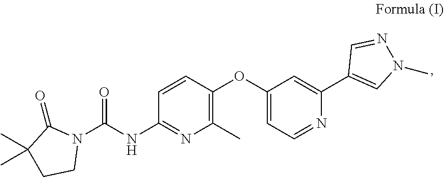

wherein the compound is present in the pharmaceutically acceptable unit formulation as a free base, or a pharmaceutically acceptable salt of the compound, and wherein the free base or pharmaceutically acceptable salt is in anhydrous or hydrate form, and one or more pharmaceutically acceptable excipients, wherein the one or more pharmaceutically acceptable excipients comprise:
(b) about 20% by weight to about 97% by weight of one or more fillers based on the weight of the pharmaceutically acceptable unit formulation;
(c) about 1% by weight to about 20% by weight of one or more disintegrants based on the weight of the pharmaceutically acceptable unit formulation; and
(d) about 0.1% by weight to about 10% by weight of one or more lubricants based on the weight of the pharmaceutically acceptable unit formulation.

Also provided herein, in some embodiments, is a pharmaceutically acceptable unit formulation comprising:
(a) about 10% by weight to about 30% by weight of the compound of Formula (I) based on the weight of the pharmaceutically acceptable unit formulation;
(b) about 70% by weight to about 90% by weight of one or more fillers based on the weight of the pharmaceutically acceptable unit formulation;
(c) about 1% by weight to about 10% by weight of one or more disintegrants based on the weight of the pharmaceutically acceptable unit formulation; and
(d) about 0.1% by weight to about 3% by weight of one or more lubricants based on the weight of the pharmaceutically acceptable unit formulation.

Also provided herein, in some embodiments, is a pharmaceutically acceptable unit formulation consisting essentially of:
(a) about 1% by weight to about 70% by weight, based on the weight of the pharmaceutically acceptable unit formulation, of a compound of Formula (I):

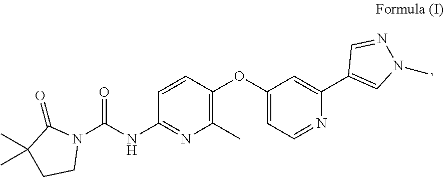

wherein the compound is present in the pharmaceutically acceptable unit formulation as a free base, or a pharmaceutically acceptable salt of the compound, and wherein the free base or pharmaceutically acceptable salt is in anhydrous or hydrate form, and one or more pharmaceutically acceptable excipients, wherein the one or more pharmaceutically acceptable excipients comprise:
(b) about 20% by weight to about 97% by weight of one or more fillers based on the weight of the pharmaceutically acceptable unit formulation;
(c) about 1% by weight to about 20% by weight of one or more disintegrants based on the weight of the pharmaceutically acceptable unit formulation; and
(d) about 0.1% by weight to about 10% by weight of one or more lubricants based on the weight of the pharmaceutically acceptable unit formulation.

Also provided herein, in some embodiments, is a pharmaceutically acceptable unit formulation consisting essentially of:
(a) about 1% by weight to about 30% by weight of a compound of Formula (I) based on the weight of the pharmaceutically acceptable unit formulation;
(b) about 20% by weight to about 90% by weight of one or more fillers based on the weight of the pharmaceutically acceptable unit formulation;
(c) about 1% by weight to about 20% by weight of one or more disintegrants based on the weight of the pharmaceutically acceptable unit formulation; and
(d) about 0.1% by weight to about 10% by weight of one or more lubricants based on the weight of the pharmaceutically acceptable unit formulation.

In some embodiments, the pharmaceutically acceptable unit formulation comprises about 1% by weight to about 99% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation comprises about 1% by weight to about 90% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation comprises about 1% by weight to about 80% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation comprises about 1% by weight to about 60% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation comprises about 1% by weight to about 50% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation comprises about 1% by weight to about 40% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation comprises about 1% by weight to about 30% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation comprises about 1% by weight to about 20% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form.

In some embodiments, the pharmaceutically acceptable unit formulation consists essentially of about 1% by weight to about 60% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation consists essentially of about 1% by weight to about 50% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation consists essentially of about 1% by weight to about 40% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation consists essentially of about 1% by weight to about 30% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form. In some embodiments, the pharmaceutically acceptable unit formulation consists essentially of about 1% by weight to about 20% by weight, based on the weight of the pharmaceutically acceptable unit formulation, of a compound of Formula (I), wherein the compound is present in the pharmaceutically acceptable formulation as a free base, or a pharmaceutically acceptable salt of the compound and wherein the free base or pharmaceutically acceptable salt is in anhydrous or hydrate form.

In some embodiments, the pharmaceutically acceptable unit formulation comprises about 1% by weight to about 70% by weight of the compound of Formula (I) based on the weight of the pharmaceutically acceptable unit formulation. In some embodiments, the pharmaceutically acceptable unit formulation comprises about 1% by weight to about 65% by weight of the compound of Formula (I) based on the weight of the pharmaceutically acceptable unit formulation. In some embodiments, the pharmaceutically acceptable unit formulation comprises about 1% by weight to about 60% by weight of the compound of Formula (I) based on the weight of the pharmaceutically acceptable unit formulation. In some embodiments, the pharmaceutically acceptable unit formulation comprises about 1% by weight to about 55% by weight of the compound of Formula (I) based on the weight of the pharmaceutically acceptable unit formulation. In some embodiments, the pharmaceutically acceptable unit formulation comprises about 1% by weight to about 50% by weight of the compound of Formula (I) based on the weight of the pharmaceutically acceptable unit formulation. In some embodiments, the pharmaceutically acceptable unit formulation comprises about 1% by weight to about 45% by weight of the compound of Formula (I) based on the weight of the pharmaceutically acceptable unit formulation. In some embodiments, the pharmaceutically acceptable unit formulation comprises about 1% by weight to about 40% by weight of the compound of Formula (I) based on the weight of the pharmaceutically acceptable unit formulation. In some embodiments, the pharmaceutically acceptable unit formulation comprises about 1% by weight to about 35% by weight of the compound of Formula (I) based on the weight of the pharmaceutically acceptable unit formulation. In some embodiments, the pharmaceutically acceptable unit formulation comprises about 1% by weight to about 30% by weight of the compound of Formula (I) based on the weight of the pharmaceutically acceptable unit formulation. In some embodiments, the pharmaceutically acceptable unit formulation comprises about 1% by weight to about 25% by weight of the compound of Formula (I) based on the weight of the pharmaceutically acceptable unit formulation. In some embodiments, the pharmaceutically acceptable unit formulation comprises about 1% by weight to about 20% by weight of the compound of Formula (I) based on the weight of the pharmaceutically acceptable unit formulation. In some embodiments, the pharmaceutically acceptable unit formulation comprises about 1% by weight to about 15% by weight of the compound of Formula (I) based on the weight of the pharmaceutically acceptable unit formulation. In some embodiments, the pharmaceutically acceptable unit formulation comprises about 1% by weight to about 10% by weight of the compound of Formula (I) based on the weight of the pharmaceutically acceptable unit formulation.

In some embodiments, the pharmaceutically acceptable unit formulation consists essentially of about 1% by weight to about 70% by weight of the compound of Formula (I) based on the weight of the pharmaceutically acceptable unit formulation. In some embodiments, the pharmaceutically acceptable unit formulation consists essentially of about 1% by weight to about 65% by weight of the compound of Formula (I) based on the weight of the pharmaceutically acceptable unit formulation. In some embodiments, the pharmaceutically acceptable unit formulation consists essentially of about 1% by weight to about 60% by weight of the compound of Formula (I) based on the weight of the pharmaceutically acceptable unit formulation. In some embodiments, the pharmaceutically acceptable unit formulation consists essentially of about 1% by weight to about 55% by weight of the compound of Formula (I) based on the weight of the pharmaceutically acceptable unit formulation. In some embodiments, the pharmaceutically acceptable unit formulation consists essentially of about 1% by weight to about 50% by weight of the compound of Formula (I) based on the weight of the pharmaceutically acceptable unit formulation. In some embodiments, the pharmaceutically acceptable unit formulation consists essentially of about 1% by weight to about 45% by weight of the compound of Formula (I) based on the weight of the pharmaceutically acceptable unit formulation. In some embodiments, the pharmaceutically acceptable unit formulation consists essentially of about 1% by weight to about 40% by weight of the compound of Formula (I) based on the weight of the pharmaceutically acceptable unit formulation. In some embodiments, the pharmaceutically acceptable unit formulation consists essentially of about 1% by weight to about 35% by weight of the compound of Formula (I) based on the weight of the pharmaceutically acceptable unit formulation. In some embodiments, the pharmaceutically acceptable unit formulation consists essentially of about 1% by weight to about 30% by weight of the compound of Formula (I) based on the weight of the pharmaceutically acceptable unit formulation. In some embodiments, the pharmaceutically acceptable unit formulation consists essentially of about 1% by weight to about 25% by weight of the compound of Formula (I) based on the weight of the pharmaceutically acceptable unit formulation. In some embodiments, the pharmaceutically acceptable unit formulation consists essentially of about 1% by weight to about 20% by weight of the compound of Formula (I) based on the weight of the pharmaceutically acceptable unit formulation. In some embodiments, the pharmaceutically acceptable unit formulation consists essentially of about 1% by weight to about 15% by weight of the compound of Formula (I) based on the weight of the pharmaceutically acceptable unit formulation. In some embodiments, the pharmaceutically acceptable unit formulation consists essentially of about 1% by weight to about 10% by weight of the compound of Formula (I) based on the weight of the pharmaceutically acceptable unit formulation.

In some embodiments, the pharmaceutically acceptable unit formulation comprises about 5% by weight to about 30% by weight of the compound of Formula (I) based on the weight of the pharmaceutically acceptable unit formulation. In some embodiments, the pharmaceutically acceptable unit formulation comprises about 5% by weight to about 25% by weight of the compound of Formula (I) based on the weight of the pharmaceutically acceptable unit formulation. In some embodiments, the pharmaceutically acceptable unit formulation comprises about 5% by weight to about 20% by weight of the compound of Formula (I) based on the weight of the pharmaceutically acceptable unit formulation. In some embodiments, the pharmaceutically acceptable unit formulation comprises about 5% by weight to about 15% by weight of the compound of Formula (I) based on the weight of the pharmaceutically acceptable unit formulation. In some embodiments, the pharmaceutically acceptable unit formulation comprises about 5% by weight to about 10% by weight of the compound of Formula (I) based on the weight of the pharmaceutically acceptable unit formulation.

In some embodiments, the pharmaceutically acceptable unit formulation consists essentially of about 5% by weight to about 30% by weight of the compound of Formula (I) based on the weight of the pharmaceutically acceptable unit formulation. In some embodiments, the pharmaceutically acceptable unit formulation consists essentially of about 5% by weight to about 25% by weight of the compound of Formula (I) based on the weight of the pharmaceutically acceptable unit formulation. In some embodiments, the pharmaceutically acceptable unit formulation consists essentially of about 5% by weight to about 20% by weight of the compound of Formula (I) based on the weight of the pharmaceutically acceptable unit formulation. In some embodiments, the pharmaceutically acceptable unit formulation consists essentially of about 5% by weight to about 15% by weight of the compound of Formula (I) based on the weight of the pharmaceutically acceptable unit formulation. In some embodiments, the pharmaceutically acceptable unit formulation consists essentially of about 5% by weight to about 10% by weight of the compound of Formula (I) based on the weight of the pharmaceutically acceptable unit formulation.

In some embodiments, the pharmaceutically acceptable unit formulation comprises about 10% to about 30% of the compound of Formula (I) based on the weight of the pharmaceutically acceptable unit formulation. In some embodiments, the pharmaceutically acceptable unit formulation comprises about 10% to about 25% of the compound of Formula (I) based on the weight of the pharmaceutically acceptable unit formulation. In some embodiments, the pharmaceutically acceptable unit formulation comprises about 10% to about 20% of the compound of Formula (I) based on the weight of the pharmaceutically acceptable unit formulation. In some embodiments, the pharmaceutically acceptable unit formulation comprises about 10% by weight to about 15% by weight of the compound of Formula (I) based on the weight of the pharmaceutically acceptable unit formulation.

In some embodiments, the pharmaceutically acceptable unit formulation consists essentially of about 10% to about 30% of the compound of Formula (I) based on the weight of the pharmaceutically acceptable unit formulation. In some embodiments, the pharmaceutically acceptable unit formulation consists essentially of about 10% to about 25% of the compound of Formula (I) based on the weight of the pharmaceutically acceptable unit formulation. In some embodiments, the pharmaceutically acceptable unit formulation consists essentially of about 10% to about 20% of the compound of Formula (I) based on the weight of the pharmaceutically acceptable unit formulation. In some embodiments, the pharmaceutically acceptable unit formulation consists essentially of about 10% by weight to about 15% by weight of the compound of Formula (I) based on the weight of the pharmaceutically acceptable unit formulation.

In some embodiments, the pharmaceutically acceptable unit formulation comprises about 1% by weight to about 30% by weight of the compound of Formula (I) based on the weight of the pharmaceutically acceptable unit formulation. In some embodiments, the pharmaceutically acceptable unit formulation comprises about 1% by weight to about 10% by weight of the compound of Formula (I) based on the weight of the pharmaceutically acceptable unit formulation. In some embodiments, the pharmaceutically acceptable unit formulation comprises about 10% to about 20% of the compound of Formula (I) based on the weight of the pharmaceutically acceptable unit formulation. In some embodiments, the pharmaceutically acceptable unit formulation comprises about 10% by weight to about 15% by weight of the compound of Formula (I) based on the weight of the pharmaceutically acceptable unit formulation.

In some embodiments, the pharmaceutically acceptable unit formulation consists essentially of about 1% by weight to about 30% by weight of the compound of Formula (I) based on the weight of the pharmaceutically acceptable unit formulation. In some embodiments, the pharmaceutically acceptable unit formulation consists essentially of about 1% by weight to about 10% by weight of the compound of Formula (I) based on the weight of the pharmaceutically acceptable unit formulation. In some embodiments, the pharmaceutically acceptable unit formulation consists essentially of about 10% to about 20% of the compound of Formula (I) based on the weight of the pharmaceutically acceptable unit formulation. In some embodiments, the pharmaceutically acceptable unit formulation consists essentially of about 10% by weight to about 15% by weight of the compound of Formula (I) based on the weight of the pharmaceutically acceptable unit formulation.

In some embodiments, the one or more pharmaceutically acceptable excipients comprise about 20% by weight to about 90% by weight of one or more fillers based on the weight of the pharmaceutically acceptable unit formulation. In some embodiments, the one or more pharmaceutically acceptable excipients comprise about 20% by weight to about 85% by weight of one or more fillers based on the weight of the pharmaceutically acceptable unit formulation. In some embodiments, the one or more pharmaceutically acceptable excipients comprise about 20% by weight to about 80% by weight of one or more fillers based on the weight of the pharmaceutically acceptable unit formulation. In some embodiments, the one or more pharmaceutically acceptable excipients comprise about 20% by weight to about 75% by weight of one or more fillers based on the weight of the pharmaceutically acceptable unit formulation. In some embodiments, the one or more pharmaceutically acceptable excipients comprise about 20% by weight to about 70% by weight of one or more fillers based on the weight of the pharmaceutically acceptable unit formulation. In some embodiments, the one or more pharmaceutically acceptable excipients comprise about 20% by weight to about 65% by weight of one or more fillers based on the weight of the pharmaceutically acceptable unit formulation. In some embodiments, the one or more pharmaceutically acceptable excipients comprise about 20% by weight to about 60% by weight of one or more fillers based on the weight of the pharmaceutically acceptable unit formulation. In some embodiments, the one or more pharmaceutically acceptable excipients comprise about 20% by weight to about 55% by weight of one or more fillers based on the weight of the pharmaceutically acceptable unit formulation. In some embodiments, the one or more pharmaceutically acceptable excipients comprise about 20% by weight to about 50% by weight of one or more fillers based on the weight of the pharmaceutically acceptable unit formulation. In some embodiments, the one or more pharmaceutically acceptable excipients comprise about 20% by weight to about 45% by weight of one or more fillers based on the weight of the pharmaceutically acceptable unit formulation. In some embodiments, the one or more pharmaceutically acceptable excipients comprise about 20% by weight to about 40% by weight of one or more fillers based on the weight of the pharmaceutically acceptable unit formulation. In some embodiments, the one or more pharmaceutically acceptable excipients comprise about 20% by weight to about 35% by weight of one or more fillers based on the weight of the pharmaceutically acceptable unit formulation. In some embodiments, the one or more pharmaceutically acceptable excipients comprise about 20% by weight to about 30% by weight of one or more fillers based on the weight of the pharmaceutically acceptable unit formulation. In some embodiments, the one or more pharmaceutically acceptable excipients comprise about 20% by weight to about 25% by weight of one or more fillers based on the weight of the pharmaceutically acceptable unit formulation.

In some embodiments, the one or more pharmaceutically acceptable excipients comprise about 30% by weight to about 90% by weight of one or more fillers based on the weight of the pharmaceutically acceptable unit formulation. In some embodiments, the one or more pharmaceutically acceptable excipients comprise about 30% by weight to about 80% by weight of one or more fillers based on the weight of the pharmaceutically acceptable unit formulation. In some embodiments, the one or more pharmaceutically acceptable excipients comprise about 30% by weight to about 70% by weight of one or more fillers based on the weight of the pharmaceutically acceptable unit formulation. In some embodiments, the one or more pharmaceutically acceptable excipients comprise about 30% by weight to about 60% by weight of one or more fillers based on the weight of the pharmaceutically acceptable unit formulation. In some embodiments, the one or more pharmaceutically acceptable excipients comprise about 30% by weight to about 50% by weight of one or more fillers based on the weight of the pharmaceutically acceptable unit formulation. In some embodiments, the one or more pharmaceutically acceptable excipients comprise about 30% by weight to about 40% by weight of one or more fillers based on the weight of the pharmaceutically acceptable unit formulation.

In some embodiments, the one or more pharmaceutically acceptable excipients comprise about 40% by weight to about 90% by weight of one or more fillers based on the weight of the pharmaceutically acceptable unit formulation. In some embodiments, the one or more pharmaceutically acceptable excipients comprise about 40% by weight to about 80% by weight of one or more fillers based on the weight of the pharmaceutically acceptable unit formulation. In some embodiments, the one or more pharmaceutically acceptable excipients comprise about 40% by weight to about 70% by weight of one or more fillers based on the weight of the pharmaceutically acceptable unit formulation. In some embodiments, the one or more pharmaceutically acceptable excipients comprise about 40% by weight to about 60% by weight of one or more fillers based on the weight of the pharmaceutically acceptable unit formulation. In some embodiments, the one or more pharmaceutically acceptable excipients comprise about 40% by weight to about 50% by weight of one or more fillers based on the weight of the pharmaceutically acceptable unit formulation.

In some embodiments, the one or more pharmaceutically acceptable excipients comprise about 50% by weight to about 90% by weight of one or more fillers based on the weight of the pharmaceutically acceptable unit formulation. In some embodiments, the one or more pharmaceutically acceptable excipients comprise about 50% by weight to about 80% by weight of one or more fillers based on the weight of the pharmaceutically acceptable unit formulation. In some embodiments, the one or more pharmaceutically acceptable excipients comprise about 50% by weight to about 70% by weight of one or more fillers based on the weight of the pharmaceutically acceptable unit formulation. In some embodiments, the one or more pharmaceutically acceptable excipients comprise about 50% by weight to about 60% by weight of one or more fillers based on the weight of the pharmaceutically acceptable unit formulation.

In some embodiments, the one or more pharmaceutically acceptable excipients comprise about 60% by weight to about 90% by weight of one or more fillers based on the weight of the pharmaceutically acceptable unit formulation. In some embodiments, the one or more pharmaceutically acceptable excipients comprise about 60% by weight to about 80% by weight of one or more fillers based on the weight of the pharmaceutically acceptable unit formulation. In some embodiments, the one or more pharmaceutically acceptable excipients comprise about 60% by weight to about 70% by weight of one or more fillers based on the weight of the pharmaceutically acceptable unit formulation.

In some embodiments, the one or more pharmaceutically acceptable excipients comprise about 70% by weight to about 90% by weight of one or more fillers based on the weight of the pharmaceutically acceptable unit formulation. In some embodiments, the one or more pharmaceutically acceptable excipients comprise about 70% by weight to about 80% by weight of one or more fillers based on the weight of the pharmaceutically acceptable unit formulation.

In some embodiments, the one or more pharmaceutically acceptable excipients comprise about 80% by weight to about 90% by weight of one or more fillers based on the weight of the pharmaceutically acceptable unit formulation.

In some embodiments, the one or more pharmaceutically acceptable excipients comprise about 1% by weight to about 20% by weight of one or more disintegrants based on the weight of the pharmaceutically acceptable unit formulation. In some embodiments, the one or more pharmaceutically acceptable excipients comprise about 1% by weight to about 15% by weight of one or more disintegrants based on the weight of the pharmaceutically acceptable unit formulation. In some embodiments, the one or more pharmaceutically acceptable excipients comprise about 1% by weight to about 14% by weight of one or more disintegrants based on the weight of the pharmaceutically acceptable unit formulation. In some embodiments, the one or more pharmaceutically acceptable excipients comprise about 1% by weight to about 13% by weight of one or more disintegrants based on the weight of the pharmaceutically acceptable unit formulation. In some embodiments, the one or more pharmaceutically acceptable excipients comprise about 1% by weight to about 12% by weight of one or more disintegrants based on the weight of the pharmaceutically acceptable unit formulation. In some embodiments, the one or more pharmaceutically acceptable excipients comprise about 1% by weight to about 11% by weight of one or more disintegrants based on the weight of the pharmaceutically acceptable unit formulation. In some embodiments, the one or more pharmaceutically acceptable excipients comprise about 1% by weight to about 10% by weight of one or more disintegrants based on the weight of the pharmaceutically acceptable unit formulation. In some embodiments, the one or more pharmaceutically acceptable excipients comprise about 1% by weight to about 9% by weight of one or more disintegrants based on the weight of the pharmaceutically acceptable unit formulation. In some embodiments, the one or more pharmaceutically acceptable excipients comprise about 1% by weight to about 8% by weight of one or more disintegrants based on the weight of the pharmaceutically acceptable unit formulation. In some embodiments, the one or more pharmaceutically acceptable excipients comprise about 1% by weight to about 7% by weight of one or more disintegrants based on the weight of the pharmaceutically acceptable unit formulation. In some embodiments, the one or more pharmaceutically acceptable excipients comprise about 1% by weight to about 6% by weight of one or more disintegrants based on the weight of the pharmaceutically acceptable unit formulation. In some embodiments, the one or more pharmaceutically acceptable excipients comprise about 1% by weight to about 5% by weight of one or more disintegrants based on the weight of the pharmaceutically acceptable unit formulation. In some embodiments, the one or more pharmaceutically acceptable excipients comprise about 1% by weight to about 4% by weight of one or more disintegrants based on the weight of the pharmaceutically acceptable unit formulation. In some embodiments, the one or more pharmaceutically acceptable excipients comprise about 1% by weight to about 3% by weight of one or more disintegrants based on the weight of the pharmaceutically acceptable unit formulation.

In some embodiments, the one or more pharmaceutically acceptable excipients comprise about 1% by weight of one or more disintegrants based on the weight of the pharmaceutically acceptable unit formulation. In some embodiments, the one or more pharmaceutically acceptable excipients comprise about 2% by weight of one or more disintegrants based on the weight of the pharmaceutically acceptable unit formulation. In some embodiments, the one or more pharmaceutically acceptable excipients comprise about 3% by weight of one or more disintegrants based on the weight of the pharmaceutically acceptable unit formulation. In some embodiments, the one or more pharmaceutically acceptable excipients comprise about 4% by weight of one or more disintegrants based on the weight of the pharmaceutically acceptable unit formulation. In some embodiments, the one or more pharmaceutically acceptable excipients comprise about 5% by weight of one or more disintegrants based on the weight of the pharmaceutically acceptable unit formulation. In some embodiments, the one or more pharmaceutically acceptable excipients comprise about 6% by weight of one or more disintegrants based on the weight of the pharmaceutically acceptable unit formulation. In some embodiments, the one or more pharmaceutically acceptable excipients comprise about 7% by weight of one or more disintegrants based on the weight of the pharmaceutically acceptable unit formulation. In some embodiments, the one or more pharmaceutically acceptable excipients comprise about 8% by weight of one or more disintegrants based on the weight of the pharmaceutically acceptable unit formulation. In some embodiments, the one or more pharmaceutically acceptable excipients comprise about 9% by weight of one or more disintegrants based on the weight of the pharmaceutically acceptable unit formulation. In some embodiments, the one or more pharmaceutically acceptable excipients comprise about 10% by weight of one or more disintegrants based on the weight of the pharmaceutically acceptable unit formulation. In some embodiments, the one or more pharmaceutically acceptable excipients comprise about 11% by weight of one or more disintegrants based on the weight of the pharmaceutically acceptable unit formulation. In some embodiments, the one or more pharmaceutically acceptable excipients comprise about 12% by weight of one or more disintegrants based on the weight of the pharmaceutically acceptable unit formulation. In some embodiments, the one or more pharmaceutically acceptable excipients comprise about 13% by weight of one or more disintegrants based on the weight of the pharmaceutically acceptable unit formulation. In some embodiments, the one or more pharmaceutically acceptable excipients comprise about 14% by weight of one or more disintegrants based on the weight of the pharmaceutically acceptable unit formulation. In some embodiments, the one or more pharmaceutically acceptable excipients comprise about 15% by weight of one or more disintegrants based on the weight of the pharmaceutically acceptable unit formulation.

In some embodiments, the one or more pharmaceutically acceptable excipients comprise about 0.1% by weight to about 10% by weight of one or more lubricants based on the weight of the pharmaceutically acceptable unit formulation. In some embodiments, the one or more pharmaceutically acceptable excipients comprise about 0.1% by weight to about 9% by weight of one or more lubricants based on the weight of the pharmaceutically acceptable unit formulation. In some embodiments, the one or more pharmaceutically acceptable excipients comprise about 0.1% by weight to about 8% by weight of one or more lubricants based on the weight of the pharmaceutically acceptable unit formulation. In some embodiments, the one or more pharmaceutically acceptable excipients comprise about 0.1% by weight to about 7% by weight of one or more lubricants based on the weight of the pharmaceutically acceptable unit formulation. In some embodiments, the one or more pharmaceutically acceptable excipients comprise about 0.1% by weight to about 6% by weight of one or more lubricants based on the weight of the pharmaceutically acceptable unit formulation. In some embodiments, the one or more pharmaceutically acceptable excipients comprise about 0.1% by weight to about 5% by weight of one or more lubricants based on the weight of the pharmaceutically acceptable unit formulation. In some embodiments, the one or more pharmaceutically acceptable excipients comprise about 0.1% by weight to about 4% by weight of one or more lubricants based on the weight of the pharmaceutically acceptable unit formulation. In some embodiments, the one or more pharmaceutically acceptable excipients comprise about 0.1% by weight to about 3% by weight of one or more lubricants based on the weight of the pharmaceutically acceptable unit formulation. In some embodiments, the one or more pharmaceutically acceptable excipients comprise about 0.1% by weight to about 2% by weight of one or more lubricants based on the weight of the pharmaceutically acceptable unit formulation.

In some embodiments, the one or more pharmaceutically acceptable excipients comprise about 0.1% by weight to about 1.9% by weight of one or more lubricants based on the weight of the pharmaceutically acceptable unit formulation. In some embodiments, the one or more pharmaceutically acceptable excipients comprise about 0.1% by weight to about 1.8% by weight of one or more lubricants based on the weight of the pharmaceutically acceptable unit formulation. In some embodiments, the one or more pharmaceutically acceptable excipients comprise about 0.1% by weight to about 1.7% by weight of one or more lubricants based on the weight of the pharmaceutically acceptable unit formulation. In some embodiments, the one or more pharmaceutically acceptable excipients comprise about 0.1% by weight to about 1.6% by weight of one or more lubricants based on the weight of the pharmaceutically acceptable unit formulation. In some embodiments, the one or more pharmaceutically acceptable excipients comprise about 0.1% by weight to about 1.5% by weight of one or more lubricants based on the weight of the pharmaceutically acceptable unit formulation. In some embodiments, the one or more pharmaceutically acceptable excipients comprise about 0.1% by weight to about 1.4% by weight of one or more lubricants based on the weight of the pharmaceutically acceptable unit formulation. In some embodiments, the one or more pharmaceutically acceptable excipients comprise about 0.1% by weight to about 1.3% by weight of one or more lubricants based on the weight of the pharmaceutically acceptable unit formulation. In some embodiments, the one or more pharmaceutically acceptable excipients comprise about 0.1% by weight to about 1.2% by weight of one or more lubricants based on the weight of the pharmaceutically acceptable unit formulation. In some embodiments, the one or more pharmaceutically acceptable excipients comprise about 0.1% by weight to about 1.1% by weight of one or more lubricants based on the weight of the pharmaceutically acceptable unit formulation. In some embodiments, the one or more pharmaceutically acceptable excipients comprise about 0.1% by weight to about 1.0% by weight of one or more lubricants based on the weight of the pharmaceutically acceptable unit formulation. In some embodiments, the one or more pharmaceutically acceptable excipients comprise about 0.1% by weight to about 0.9% by weight of one or more lubricants based on the weight of the pharmaceutically acceptable unit formulation. In some embodiments, the one or more pharmaceutically acceptable excipients comprise about 0.1% by weight to about 0.8% by weight of one or more lubricants based on the weight of the pharmaceutically acceptable unit formulation. In some embodiments, the one or more pharmaceutically acceptable excipients comprise about 0.1% by weight to about 0.7% by weight of one or more lubricants based on the weight of the pharmaceutically acceptable unit formulation. In some embodiments, the one or more pharmaceutically acceptable excipients comprise about 0.1% by weight to about 0.6% by weight of one or more lubricants based on the weight of the pharmaceutically acceptable unit formulation. In some embodiments, the one or more pharmaceutically acceptable excipients comprise about 0.1% by weight to about 0.5% by weight of one or more lubricants based on the weight of the pharmaceutically acceptable unit formulation. In some embodiments, the one or more pharmaceutically acceptable excipients comprise about 0.1% by weight to about 0.4% by weight of one or more lubricants based on the weight of the pharmaceutically acceptable unit formulation. In some embodiments, the one or more pharmaceutically acceptable excipients comprise about 0.1% by weight to about 0.3% by weight of one or more lubricants based on the weight of the pharmaceutically acceptable unit formulation. In some embodiments, the one or more pharmaceutically acceptable excipients comprise about 0.1% by weight to about 0.2% by weight of one or more lubricants based on the weight of the pharmaceutically acceptable unit formulation.

In some embodiments, the one or more pharmaceutically acceptable excipients comprise about 0.1% by weight of one or more lubricants based on the weight of the pharmaceutically acceptable unit formulation. In some embodiments, the one or more pharmaceutically acceptable excipients comprise about 0.2% by weight of one or more lubricants based on the weight of the pharmaceutically acceptable unit formulation. In some embodiments, the one or more pharmaceutically acceptable excipients comprise about 0.3% by weight of one or more lubricants based on the weight of the pharmaceutically acceptable unit formulation. In some embodiments, the one or more pharmaceutically acceptable excipients comprise about 0.4% by weight of one or more lubricants based on the weight of the pharmaceutically acceptable unit formulation. In some embodiments, the one or more pharmaceutically acceptable excipients comprise about 0.5% by weight of one or more lubricants based on the weight of the pharmaceutically acceptable unit formulation. In some embodiments, the one or more pharmaceutically acceptable excipients comprise about 0.6% by weight of one or more lubricants based on the weight of the pharmaceutically acceptable unit formulation. In some embodiments, the one or more pharmaceutically acceptable excipients comprise about 0.7% by weight of one or more lubricants based on the weight of the pharmaceutically acceptable unit formulation. In some embodiments, the one or more pharmaceutically acceptable excipients comprise about 0.8% by weight of one or more lubricants based on the weight of the pharmaceutically acceptable unit formulation. In some embodiments, the one or more pharmaceutically acceptable excipients comprise about 0.9% by weight of one or more lubricants based on the weight of the pharmaceutically acceptable unit formulation. In some embodiments, the one or more pharmaceutically acceptable excipients comprise about 1.0% by weight of one or more lubricants based on the weight of the pharmaceutically acceptable unit formulation.

In some embodiments, the pharmaceutically acceptable unit formulation comprises: (a) about 1% by weight to about 30% by weight of the compound of Formula (I) based on the weight of the pharmaceutically acceptable unit formulation; (b) about 20% by weight to about 90% by weight of one or more fillers based on the weight of the pharmaceutically acceptable unit formulation; (c) about 1% by weight to about 20% by weight of one or more disintegrants based on the weight of the pharmaceutically acceptable unit formulation; and (d) about 0.1% by weight to about 10% by weight of one or more lubricants based on the weight of the pharmaceutically acceptable unit formulation.

In some embodiments, the pharmaceutically acceptable unit formulation consists essentially of: (a) about 1% by weight to about 30% by weight of the compound of Formula (I) based on the weight of the pharmaceutically acceptable unit formulation; (b) about 20% by weight to about 90% by weight of one or more fillers based on the weight of the pharmaceutically acceptable unit formulation; (c) about 1% by weight to about 20% by weight of one or more disintegrants based on the weight of the pharmaceutically acceptable unit formulation; and (d) about 0.1% by weight to about 10% by weight of one or more lubricants based on the weight of the pharmaceutically acceptable unit formulation.

In some embodiments, the pharmaceutically acceptable unit formulation comprises (a) about 5% by weight to about 25% by weight of the compound of Formula (I) based on the weight of the pharmaceutically acceptable unit formulation; (b) about 60% by weight to about 90% by weight of one or more fillers based on the weight of the pharmaceutically acceptable unit formulation; (c) about 1% by weight to about 15% by weight of one or more disintegrants based on the weight of the pharmaceutically acceptable unit formulation; and (d) about 0.1% by weight to about 4% by weight of one or more lubricants based on the weight of the pharmaceutically acceptable unit formulation.

In some embodiments, the pharmaceutically acceptable unit formulation consists essentially of: (a) about 5% by weight to about 25% by weight of the compound of Formula (I) based on the weight of the pharmaceutically acceptable unit formulation; (b) about 60% by weight to about 90% by weight of one or more fillers based on the weight of the pharmaceutically acceptable unit formulation; (c) about 1% by weight to about 15% by weight of one or more disintegrants based on the weight of the pharmaceutically acceptable unit formulation; and (d) about 0.1% by weight to about 4% by weight of one or more lubricants based on the weight of the pharmaceutically acceptable unit formulation.

In some embodiments, the pharmaceutically acceptable unit formulation comprises (a) about 10% by weight to about 30% by weight of the compound of Formula (I) based on the weight of the pharmaceutically acceptable unit formulation; (b) about 70% by weight to about 90% by weight of one or more fillers based on the weight of the pharmaceutically acceptable unit formulation; (c) about 1% by weight to about 10% by weight of one or more disintegrants based on the weight of the pharmaceutically acceptable unit formulation; and (d) about 0.1% by weight to about 3% by weight of one or more lubricants based on the weight of the pharmaceutically acceptable unit formulation.

In some embodiments, the pharmaceutically acceptable unit formulation consists essentially of: (a) about 10% by weight to about 30% by weight of the compound of Formula (I) based on the weight of the pharmaceutically acceptable unit formulation; (b) about 70% by weight to about 90% by weight of one or more fillers based on the weight of the pharmaceutically acceptable unit formulation; (c) about 1% by weight to about 10% by weight of one or more disintegrants based on the weight of the pharmaceutically acceptable unit formulation; and (d) about 0.1% by weight to about 3% by weight of one or more lubricants based on the weight of the pharmaceutically acceptable unit formulation.

In some embodiments, the one or more pharmaceutically acceptable excipients is one or more fillers selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc. In some embodiments, the one or more fillers is lactose monohydrate.

In some embodiments, the one or more disintegrants is one or more disintegrants selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone (also referred to as crospovidone).

In some embodiments, the one or more lubricants is one or more lubricants selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, copolymer comprising poly(ethylene oxide) and poly(propylene oxide) (such as poloxomer 188), copolymer comprising polypropylene glycol and polyethylene glycol (such as poloxomer 407), sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate.

In some embodiments, the pharmaceutically acceptable unit formulation further comprises cross-linked polyvinylpyrrolidone and magnesium stearate. In some embodiments, the one or more pharmaceutically acceptable excipients is lactose monohydrate and the formulation further comprises cross-linked polyvinylpyrrolidone and magnesium stearate.

In some embodiments, the pharmaceutically acceptable unit formulation is for oral administration.

In some embodiments, the pharmaceutically acceptable unit formulation is a solid dosage form.

In some embodiments, the pharmaceutically acceptable unit formulation is a tablet or capsule. In some embodiments, the pharmaceutically acceptable unit formulation is a tablet. In some embodiments, the pharmaceutically acceptable unit formulation is a capsule. In some embodiments, the pharmaceutically acceptable unit formulation is a sachet. In some embodiments, the pharmaceutically acceptable unit formulation is a powder in a packet.

In some embodiments, the pharmaceutically acceptable unit formulation comprises about 1 mg to about 40 mg of the compound of Formula (I). In some embodiments, the pharmaceutically acceptable unit formulation comprises about 1 mg to about 30 mg of the compound of Formula (I). In some embodiments, the pharmaceutically acceptable unit formulation comprises about 1 mg to about 20 mg of the compound of Formula (I). In some embodiments, the pharmaceutically acceptable unit formulation comprises about 1 mg to about 15 mg of the compound of Formula (I). In some embodiments, the pharmaceutically acceptable unit formulation comprises about 5 mg to about 40 mg of the compound of Formula (I). In some embodiments, the pharmaceutically acceptable unit formulation comprises about 5 mg to about 30 mg of the compound of Formula (I). In some embodiments, the pharmaceutically acceptable unit formulation comprises about 5 mg to about 20 mg of the compound of Formula (I). In some embodiments, the pharmaceutically acceptable unit formulation comprises about 5 mg to about 15 mg of the compound of Formula (I). In some embodiments, the pharmaceutically acceptable unit formulation comprises about 10 mg to about 40 mg of the compound of Formula (I). In some embodiments, the pharmaceutically acceptable unit formulation comprises about 10 mg to about 30 mg of the compound of Formula (I). In some embodiments, the pharmaceutically acceptable unit formulation comprises about 10 mg to about 20 mg of the compound of Formula (I). In some embodiments, the pharmaceutically acceptable unit formulation comprises about 10 mg to about 15 mg of the compound of Formula (I).

In some embodiments, the pharmaceutically acceptable unit formulation consists essentially of about 1 mg to about 40 mg of the compound of Formula (I). In some embodiments, the pharmaceutically acceptable unit formulation consists essentially of about 1 mg to about 30 mg of the compound of Formula (I). In some embodiments, the pharmaceutically acceptable unit formulation consists essentially of about 1 mg to about 20 mg of the compound of Formula (I). In some embodiments, the pharmaceutically acceptable unit formulation consists essentially of about 1 mg to about 15 mg of the compound of Formula (I). In some embodiments, the pharmaceutically acceptable unit formulation consists essentially of about 5 mg to about 40 mg of the compound of Formula (I). In some embodiments, the pharmaceutically acceptable unit formulation consists essentially of about 5 mg to about 30 mg of the compound of Formula (I). In some embodiments, the pharmaceutically acceptable unit formulation consists essentially of about 5 mg to about 20 mg of the compound of Formula (I). In some embodiments, the pharmaceutically acceptable unit formulation consists essentially of about 5 mg to about 15 mg of the compound of Formula (I). In some embodiments, the pharmaceutically acceptable unit formulation consists essentially of about 10 mg to about 40 mg of the compound of Formula (I). In some embodiments, the pharmaceutically acceptable unit formulation consists essentially of about 10 mg to about 30 mg of the compound of Formula (I). In some embodiments, the pharmaceutically acceptable unit formulation consists essentially of about 10 mg to about 20 mg of the compound of Formula (I). In some embodiments, the pharmaceutically acceptable unit formulation consists essentially of about 10 mg to about 15 mg of the compound of Formula (I).

In some embodiments, compound of Formula (I) is present in the pharmaceutically acceptable unit formulation as a cocrystal. In some embodiments, compound of Formula (I) is present in the pharmaceutically acceptable unit formulation as a channel crystal. In some embodiments, the compound of Formula (I) is present in the pharmaceutically acceptable unit formulation as a crystalline anhydrous form. In some embodiments, compound of Formula (I) is present in the pharmaceutically acceptable unit formulation as a solvate.

In some embodiments, the pharmaceutically acceptable unit formulation has an acceptance value of less than about 15 as determined by USP Uniformity of Dosage Unit <905>. In some embodiments, the pharmaceutically acceptable unit formulation has a maximum acceptance value of equal to or less than about 15 as determined by USP Uniformity of Dosage Unit <905>.

In some embodiments, the uniformity of the pharmaceutically acceptable formulation represents the degree of uniformity in the amount of the compound of Formula (I) in the pharmaceutically acceptable formulation.

In some embodiments, the uniformity of the oral dosage form represents the degree of uniformity in the amount of the compound of Formula (I) in the oral dosage form.

In some embodiments, the uniformity of the pharmaceutically acceptable capsule represents the degree of uniformity in the amount of the compound of Formula (I) in the pharmaceutically acceptable capsule.

In one embodiment of the disclosure, pharmaceutically acceptable formulation comprising a compound of Formula (I):

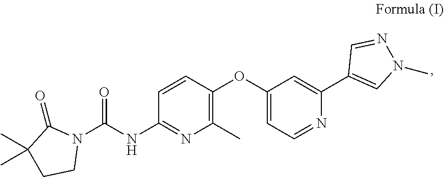

and one or more pharmaceutically acceptable excipients, wherein the compound is present in the pharmaceutically acceptable formulation as a free base, or a pharmaceutically acceptable salt of the compound and wherein the free base or pharmaceutically acceptable salt is in anhydrous or hydrate form.

In some embodiments, the compound of Formula (I) is present in the pharmaceutically acceptable formulation as a hydrate of a pharmaceutically acceptable salt. In some embodiments, the compound of Formula (I) is present in the pharmaceutically acceptable formulation as a monohydrate or dihydrate of a pharmaceutically acceptable salt. In some embodiments, the compound of Formula (I) is present in the pharmaceutically acceptable formulation as an anhydrous form or a monohydrate of a hydrochloride salt. In some embodiments, the compound of Formula (I) is present in the pharmaceutically acceptable formulation as a monohydrate of a hydrochloride salt. In some embodiments, the compound of Formula (I) is present in the pharmaceutically acceptable formulation as a dihydrate of a hydrochloride salt. In some embodiments, the one or more pharmaceutically acceptable excipients is a filler. In some embodiments, the one or more pharmaceutically acceptable excipients is lactose or lactose monohydrate. In some embodiments, the formulation further comprises one or more disintegrants, one or more lubricants, or a combination thereof. In some embodiments, the formulation further comprises one or more disintegrants. In some embodiments, the formulation further comprises one or more lubricants.

In another embodiment of the disclosure, provided is a pharmaceutically acceptable unit formulation comprising: (a) about 1% by weight to about 70% by weight, based on the weight of the pharmaceutically acceptable unit formulation, of a compound of Formula (I):

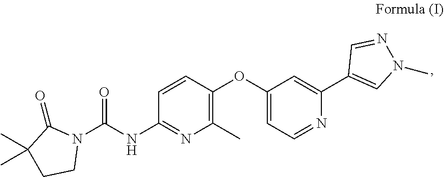

wherein the compound is present in the pharmaceutically acceptable unit formulation as a free base, or a pharmaceutically acceptable salt of the compound and wherein the free base or pharmaceutically acceptable salt is in anhydrous or hydrate form, and one or more pharmaceutically acceptable excipients, wherein the one or more pharmaceutically acceptable excipients comprise:
(b) about 20% by weight to about 97% by weight of one or more fillers based on the weight of the pharmaceutically acceptable unit formulation;
(c) about 1% by weight to about 20% by weight of one or more disintegrants based on the weight of the pharmaceutically acceptable unit formulation; and
(d) about 0.1% by weight to about 10% by weight of one or more lubricants based on the weight of the pharmaceutically acceptable unit formulation.

In another embodiment of the disclosure, the unit formulation comprising:
(a) about 10% by weight to about 30% by weight of the compound of Formula (I) based on the weight of the pharmaceutically acceptable unit formulation;
(b) about 70% by weight to about 90% by weight of one or more fillers based on the weight of the pharmaceutically acceptable unit formulation;
(c) about 1% by weight to about 10% by weight of one or more disintegrants based on the weight of the pharmaceutically acceptable unit formulation; and
(d) about 0.1% by weight to about 3% by weight of one or more lubricants based on the weight of the pharmaceutically acceptable unit formulation.

In some embodiments, the one or more fillers is selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc. In some embodiments, the one or more fillers is lactose monohydrate. In some embodiments, the one or more disintegrants is selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone. In some embodiments, the one or more lubricants is selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate. In some embodiments, the one or more pharmaceutically acceptable excipients is lactose monohydrate and the pharmaceutically acceptable unit formulation further comprises cross-linked polyvinylpyrrolidone and magnesium stearate. In some embodiments, the pharmaceutically acceptable unit formulation further comprises cross-linked polyvinylpyrrolidone and magnesium stearate. In some embodiments, the pharmaceutically acceptable unit formulation is for oral administration. In some embodiments, the pharmaceutically acceptable unit formulation is a solid dosage form. In some embodiments, the pharmaceutically acceptable unit formulation is a tablet or a capsule. In some embodiments, the pharmaceutically acceptable unit formulation is a capsule. In some embodiments, the pharmaceutically acceptable unit formulation comprises about 1% by weight to about 30% by weight of the compound of Formula (I) based on the weight of the pharmaceutically acceptable unit formulation. In some embodiments, the pharmaceutically acceptable unit formulation comprises about 1% by weight to about 10% by weight of the compound of Formula (I) based on the weight of the pharmaceutically acceptable unit formulation. In some embodiments, the pharmaceutically acceptable unit formulation comprises about 10% to about 20% of the compound of Formula (I) based on the weight of the pharmaceutically acceptable unit formulation. In some embodiments, the pharmaceutically acceptable unit formulation comprises about 10% by weight to about 15% by weight of the compound of Formula (I) based on the weight of the pharmaceutically acceptable unit formulation. In some embodiments, the pharmaceutically acceptable unit formulation comprises about 1 mg to about 60 mg of the compound of Formula (I). In some embodiments, the compound of Formula (I) is present in the pharmaceutically acceptable unit formulation as a crystalline pharmaceutically acceptable salt selected from the group consisting of a crystalline hydrochloride salt, a crystalline sulfate salt, a crystalline phosphate salt, a crystalline mesylate salt, a crystalline citrate salt, a crystalline malate salt, and a crystalline tartrate salt, wherein the crystalline pharmaceutically acceptable salt is in anhydrous or hydrate form. In some embodiments, the pharmaceutically acceptable unit formulation comprises about 27.2 mg or about 54.3 mg of the anhydrous form of the hydrochloride salt of the compound of Formula (I). In some embodiments, the pharmaceutically acceptable unit formulation comprises about 28.2 mg or about 56.5 mg of a crystalline monohydrate form of the hydrochloride salt of the compound of Formula (I). In some embodiments, the pharmaceutically acceptable unit formulation comprises about 29.3 mg or about 58.6 mg of a crystalline dihydrate form of the hydrochloride salt of the compound of Formula (I). In some embodiments, the compound of Formula (I) is present in the pharmaceutically acceptable unit formulation as a crystalline anhydrous form. In some embodiments, the pharmaceutically acceptable unit formulation comprises about 25 mg, about 50 mg, about 12.5 mg, about 10 mg, or about 5 mg of the crystalline anhydrous form of the compound of Formula (I). In some embodiments, the pharmaceutically acceptable unit formulation comprises about 27.2 mg, about 54.3 mg, about 13.6 mg, about 10.9 mg, or about 5.4 mg of a crystalline anhydrous hydrochloride salt of the compound of Formula (I). In some embodiments, the pharmaceutically acceptable unit formulation comprises about 28.2 mg, about 56.5 mg, about 14.1 mg, about 11.3 mg, or about 5.6 mg of a crystalline monohydrate of a hydrochloride salt of the compound of Formula (I). In some embodiments, the pharmaceutically acceptable unit formulation comprises about 29.3 mg, about 58.6 mg, about 14.7 mg, about 11.7 mg, or about 5.9 mg of a crystalline dihydrate hydrochloride salt of the compound of Formula (I). In some embodiments, the pharmaceutically acceptable unit formulation has an acceptance value of less than about 15 as determined by United States Pharmacopeia (USP) Uniformity of Dosage Unit <905>.

In another embodiment of the disclosure, provided herein is a pharmaceutically acceptable unit formulation comprising the crystalline hydrochloride salt of a compound of Formula (I):

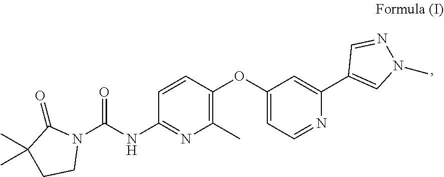

wherein the hydrochloride salt is in anhydrous or hydrate form, and one or more pharmaceutically acceptable excipients.

In some embodiments, the pharmaceutically acceptable unit formulation has not more than about 5 mol %, not more than about 3 mol %, or not more than about 1 mol % of other solid-state forms of the compound represented by Formula (I). In some embodiments, the crystalline hydrochloride salt of the compound of Formula (I), in monohydrate form, has an X-ray powder diffraction pattern comprising peaks, in terms of 2-theta, at about 9.5°, 10.60, and 19.7° as measured by CuKα radiation. In some embodiments, the crystalline hydrochloride salt of the compound of Formula (I), in monohydrate form, has an X-ray powder diffraction pattern comprising peaks, in terms of 2-theta, at about 9.5°, 10.6°, 16.6°, 18.8°, and 19.7° as measured by CuKα radiation. In some embodiments, the one or more pharmaceutically acceptable excipients is a filler. In some embodiments, the one or more pharmaceutically acceptable excipients is lactose monohydrate. In some embodiments, the pharmaceutically acceptable unit formulation further comprises one or more disintegrants, one or more lubricants, or a combination thereof. In some embodiments, the pharmaceutically acceptable unit formulation further comprises one or more disintegrants. In some embodiments, the pharmaceutically acceptable unit formulation further comprises one or more lubricants.

In another embodiment of the disclosure, provided herein is a pharmaceutically acceptable unit formulation comprising:
(a) about 1% by weight to about 70% by weight, based on the weight of the pharmaceutically acceptable unit formulation, of a crystalline hydrochloride salt of a compound of Formula (I):

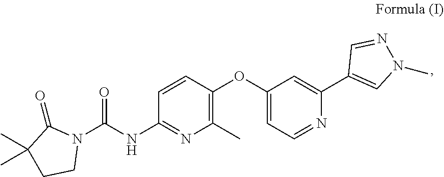

wherein the crystalline hydrochloride salt is in anhydrous or hydrate form, and one or more pharmaceutically acceptable excipients, wherein the one or more pharmaceutically acceptable excipients comprise:
(b) about 20% by weight to about 97% by weight of one or more fillers based on the weight of the pharmaceutically acceptable unit formulation;
(c) about 1% by weight to about 20% by weight of one or more disintegrants based on the weight of the pharmaceutically acceptable unit formulation; and
(d) about 0.1% by weight to about 10% by weight of one or more lubricants based on the weight of the pharmaceutically acceptable unit formulation.

In some embodiments, the pharmaceutically acceptable unit formulation further comprises:
(a) about 10% by weight to about 30% by weight of the crystalline hydrochloride salt, or a monohydrate form thereof, of the compound of Formula (I) based on the weight of the pharmaceutically acceptable unit formulation;
(b) about 70% by weight to about 90% by weight of one or more fillers based on the weight of the pharmaceutically acceptable unit formulation;
I about 1% by weight to about 10% by weight of one or more disintegrants based on the weight of the pharmaceutically acceptable unit formulation; and
(d) about 0.1% by weight to about 3% by weight of one or more lubricants based on the weight of the pharmaceutically acceptable unit formulation.

In some embodiments, the pharmaceutically acceptable unit formulation has not more than about 5 mol %, not more than about 3 mol %, or not more than about 1 mol % of other solid-state forms of the compound represented by Formula (I). In some embodiments, the one or more fillers is selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc. In some embodiments, the one or more fillers is lactose monohydrate. In some embodiments, the one or more disintegrants is selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone. In some embodiments, the one or more lubricants is one or more lubricants selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate. In some embodiments, the one or more pharmaceutically acceptable excipients is lactose monohydrate and the pharmaceutically acceptable unit formulation further comprises cross-linked polyvinylpyrrolidone and magnesium stearate. In some embodiments, the pharmaceutically acceptable unit formulation further comprises cross-linked polyvinylpyrrolidone and magnesium stearate. In some embodiments, the pharmaceutically acceptable unit formulation comprises about 1% by weight to about 30% by weight of the crystalline hydrochloride salt, of the compound of Formula (I), in anhydrous or monohydrate form, based on the weight of the pharmaceutically acceptable unit formulation. In some embodiments, the pharmaceutically acceptable unit formulation comprises about 1% by weight to about 10% by weight of the crystalline hydrochloride salt, of the compound of Formula (I), in anhydrous or monohydrate form, based on the weight of the pharmaceutically acceptable unit formulation. In some embodiments, the pharmaceutically acceptable unit formulation comprises about 10% by weight to about 20% by weight of the crystalline hydrochloride salt of the compound of Formula (I), in anhydrous or monohydrate form, based on the weight of the pharmaceutically acceptable unit formulation. In some embodiments, the pharmaceutically acceptable unit formulation comprises about 10% by weight to about 15% by weight of the crystalline hydrochloride salt of the compound of Formula (I), in anhydrous or monohydrate form, based on the weight of the pharmaceutically acceptable unit formulation. In some embodiments, the pharmaceutically acceptable unit formulation comprises about 1 mg to about 60 mg of the crystalline hydrochloride salt, in anhydrous or monohydrate form. In some embodiments, the pharmaceutically acceptable unit formulation comprises about 20 mg to about 60 mg of the crystalline hydrochloride salt, in anhydrous or monohydrate form. In some embodiments, the pharmaceutically acceptable unit formulation comprises the anhydrous form of the crystalline hydrochloride salt of the compound of Formula (I) in an amount of about 27.2 mg. In some embodiments, the pharmaceutically acceptable unit formulation comprises the anhydrous form of the crystalline hydrochloride salt of the compound of Formula (I) in an amount of about 54.3 mg. In some embodiments, the crystalline monohydrate form of the hydrochloride salt of the compound of Formula (I) is present in an amount of about 28.2 mg. In some embodiments, the crystalline monohydrate form of the hydrochloride salt of the compound of Formula (I) is present in an amount of about 56.5 mg. In some embodiments, the pharmaceutically acceptable unit formulation comprises the anhydrous form of the crystalline hydrochloride salt of the compound of Formula (I) in an amount sufficient to provide 25 mg of the compound of Formula (I). In some embodiments, the pharmaceutically acceptable unit formulation comprises the anhydrous form of the crystalline hydrochloride salt of the compound of Formula (I) in an amount sufficient to provide 50 mg of the compound of Formula (I). In some embodiments, the pharmaceutically acceptable unit formulation comprises the monohydrate form of the crystalline hydrochloride salt of the compound of Formula (I) in an amount sufficient to provide 25 mg of the compound of Formula (I). In some embodiments, the pharmaceutically acceptable unit formulation comprises the monohydrate form of the crystalline hydrochloride salt of the compound of Formula (I) in an amount sufficient to provide 50 mg of the compound of Formula (I). In some embodiments, the pharmaceutically acceptable unit formulation comprises the dihydrate form of the crystalline hydrochloride salt of the compound of Formula (I) in an amount sufficient to provide 25 mg of the compound of Formula (I). In some embodiments, the pharmaceutically acceptable unit formulation comprises the dihydrate form of the crystalline hydrochloride salt of the compound of Formula (I) in an amount sufficient to provide 50 mg of the compound of Formula (I). In some embodiments, the one or more disintegrants is selected from the group consisting of cross-linked polyvinylpyrrolidone, croscarmellose sodium, and sodium starch glycolate; and the one or more lubricants is selected from the group consisting of sodium stearyl fumarate, stearic acid, talc, calcium stearate, magnesium stearate, solid polyethylene glycols, and sodium lauryl sulfate. In some embodiments, the pharmaceutically acceptable unit formulation is for oral administration. In some embodiments, the pharmaceutically acceptable unit formulation is a solid dosage form. In some embodiments, the pharmaceutically acceptable unit formulation is a tablet or a capsule. In some embodiments, the pharmaceutically acceptable unit formulation is a capsule. In some embodiments, the pharmaceutically acceptable unit formulation has an acceptance value of less than about 15 as determined by United States Pharmacopeia (USP) Uniformity of Dosage Unit <905>.

In another embodiment, provided herein is a oral dosage form comprising a compound of Formula (I):

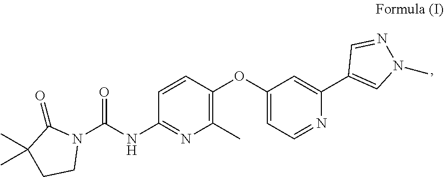

and one or more pharmaceutically acceptable excipients, wherein the compound is present in the oral dosage form as a free base, or a pharmaceutically acceptable salt of the compound and wherein the free base or pharmaceutically acceptable salt is in anhydrous or hydrate form. In some embodiments, the compound of Formula (I) is present in the oral dosage form as a pharmaceutically acceptable salt in anhydrous or a monohydrate form.

In some embodiments, the compound of Formula (I) is present in the oral dosage form as an anhydrous form. In some embodiments, the one or more pharmaceutically acceptable excipients is a filler. In some embodiments, the one or more pharmaceutically acceptable excipients is lactose monohydrate. In some embodiments, the oral dosage form further comprises one or more disintegrants, one or more lubricants, or a combination thereof. In some embodiments, the oral dosage form further comprises one or more disintegrants. In some embodiments, the oral dosage form further comprises one or more lubricants.

In an embodiment, described herein is a oral dosage form comprising:
(a) about 1% by weight to about 70% by weight, based on the total weight of the oral dosage form, of a compound of Formula (I):

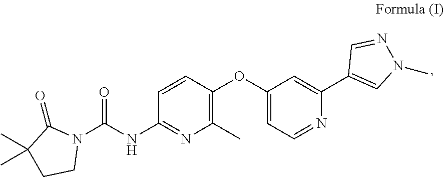

wherein the compound is present in the oral dosage form as a free base, or a pharmaceutically acceptable salt of the compound and wherein the free base or pharmaceutically acceptable salt is in anhydrous or hydrate form, and one or more pharmaceutically acceptable excipients, wherein the one or more pharmaceutically acceptable excipients comprise:
(b) about 20% by weight to about 97% by weight of one or more fillers based on the total weight of the oral dosage form; (c) about 1% by weight to about 20% by weight of one or more disintegrants based on the total weight of the oral dosage form; and
(d) about 0.1% by weight to about 10% by weight of one or more lubricants based on the total weight of the oral dosage form.

In some embodiments, the oral dosage form comprises:
(a) about 10% by weight to about 30% by weight of the compound of Formula (I) based on the total weight of the oral dosage form;
(b) about 70% by weight to about 90% by weight of one or more fillers based on the total weight of the oral dosage form;
(c) about 1% by weight to about 10% by weight of one or more disintegrants based on the total weight of the oral dosage form; and
(d) about 0.1% by weight to about 3% by weight of one or more lubricants based on the total weight of the oral dosage form.

In some embodiments, the one or more pharmaceutically acceptable excipients is one or more fillers selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc. In some embodiments, the one or more fillers is lactose monohydrate. In some embodiments, the one or more disintegrants is one or more disintegrants selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone. In some embodiments, the one or more lubricants is one or more lubricants selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate. In some embodiments, the one or more pharmaceutically acceptable excipients is lactose monohydrate and the oral dosage form further comprises cross-linked polyvinylpyrrolidone and magnesium stearate. In some embodiments, the oral dosage form further comprises cross-linked polyvinylpyrrolidone and magnesium stearate. In some embodiments, the oral dosage form comprises about 1% by weight to about 30% by weight of the compound of Formula (I) based on the total weight of the oral dosage form. In some embodiments, the oral dosage form comprises about 1% by weight to about 10% by weight of the compound of Formula (I) based on the total weight of the oral dosage form. In some embodiments, the oral dosage form comprises about 10% to about 20% of the compound of Formula (I) based on the total weight of the oral dosage form. In some embodiments, the oral dosage form comprises about 10% by weight to about 15% by weight of the compound of Formula (I) based on the total weight of the oral dosage form. In some embodiments, the oral dosage form comprises about 1 mg to about 40 mg of the compound of Formula (I). In some embodiments, the compound of Formula (I) is present in the oral dosage form as a crystalline hydrochloride salt in anhydrous or monohydrate form. In some embodiments, the oral dosage form comprises about 27.2 mg or about 54.3 mg of the anhydrous form of the crystalline hydrochloride salt of the compound of Formula (I). In some embodiments, the oral dosage form comprises about 28.2 mg or about 56.5 mg of the monohydrate form of the crystalline hydrochloride salt of the compound of Formula (I). In some embodiments, the oral dosage form comprises the anhydrous form of the crystalline hydrochloride salt of the compound of Formula (I) in an amount sufficient to provide 25 mg of the compound of Formula (I). In some embodiments, the oral dosage form comprises the anhydrous form of the crystalline hydrochloride salt of the compound of Formula (I) in an amount sufficient to provide 50 mg of the compound of Formula (I). In some embodiments, the oral dosage form comprises the monohydrate form of the crystalline hydrochloride salt of the compound of Formula (I) in an amount sufficient to provide 25 mg of the compound of Formula (I). In some embodiments, the oral dosage form comprises the monohydrate form of the crystalline hydrochloride salt of the compound of Formula (I) in an amount sufficient to provide 50 mg of the compound of Formula (I). In some embodiments, the compound of Formula (I) is present in the oral dosage form as a crystalline anhydrous form. In some embodiments, the oral dosage form comprises about 25 mg, or about 50 mg of the crystalline anhydrous form of the compound of Formula (I). In some embodiments, the oral dosage form is a tablet or a capsule. In some embodiments, the oral dosage form is a capsule.

In an embodiment, described herein is an oral dosage form comprising the crystalline hydrochloride salt of a compound of Formula (I):

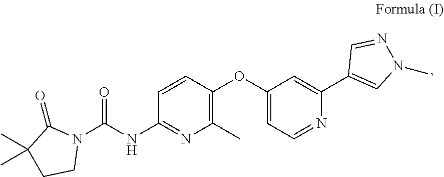

in anhydrous or monohydrate form, and one or more pharmaceutically acceptable excipients. In some embodiments, the oral dosage form has not more than about 5 mol %, not more than about 3 mol %, or not more than about 1 mol % of other solid-state forms of the compound represented by Formula (I). In some embodiments, the crystalline hydrochloride salt of the compound of Formula (I), in monohydrate form, has an X-ray powder diffraction pattern comprising peaks, in terms of 2-theta, at about 0.5°, 10.6°, and 19.7° as measured by CuKα radiation. In some embodiments, the crystalline hydrochloride salt of the compound of Formula (I), in monohydrate form has an X-ray powder diffraction pattern comprising peaks, in terms of 2-theta, at about 9.5°, 10.6°, 16.6°, 18.8°, and 19.7° as measured by CuKα radiation. In some embodiments, the one or more pharmaceutically acceptable excipients is a filler. In some embodiments, the one or more pharmaceutically acceptable excipients is lactose monohydrate. In some embodiments, the oral dosage form further comprises one or more disintegrants, one or more lubricants, or a combination thereof. In some embodiments, the oral dosage form further comprises one or more disintegrants. In some embodiments, the oral dosage form further comprises one or more lubricants.

In an embodiment, also provided is an oral dosage form comprising:
(a) about 1% by weight to about 70% by weight, based on the total weight of the oral dosage form, of a crystalline hydrochloride salt of a compound of Formula (I):

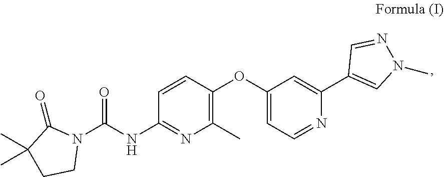

in anhydrous or monohydrate form, and one or more pharmaceutically acceptable excipients, wherein the one or more pharmaceutically acceptable excipients comprise:
(b) about 20% by weight to about 97% by weight of one or more fillers based on the total weight of the oral dosage form;
(c) about 1% by weight to about 20% by weight of one or more disintegrants based on the total weight of the oral dosage form; and
(d) about 0.1% by weight to about 10% by weight of one or more lubricants based on the total weight of the oral dosage form.

In some embodiments, the oral dosage form comprises:
(a) about 10% by weight to about 30% by weight of the crystalline hydrochloride salt of the compound of Formula (I) based on the total weight of the oral dosage form;
(b) about 70% by weight to about 90% by weight of one or more fillers based on the total weight of the oral dosage form;
(c) about 1% by weight to about 10% by weight of one or more disintegrants based on the total weight of the oral dosage form; and
(d) about 0.1% by weight to about 3% by weight of one or more lubricants based on the total weight of the oral dosage form.

In some embodiments, the oral dosage form has not more than about 5 mol %, not more than about 3 mol %, or not more than about 1 mol % of other solid-state forms of the compound represented by Formula (I). In some embodiments, the one or more pharmaceutically acceptable excipients is one or more fillers selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc.

In some embodiments, the one or more fillers is lactose monohydrate. In some embodiments, the one or more disintegrants is one or more disintegrants selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone. In some embodiments, the one or more lubricants is one or more lubricants selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate. In some embodiments, the one or more pharmaceutically acceptable excipients is lactose monohydrate and oral dosage form further comprises cross-linked polyvinylpyrrolidone and magnesium stearate. In some embodiments, the oral dosage form further comprises cross-linked polyvinylpyrrolidone and magnesium stearate. In some embodiments, the oral dosage form comprises about 1% by weight to about 30% by weight of the crystalline hydrochloride salt of the compound of Formula (I), in anhydrous or monohydrate form, based on the total weight of the oral dosage form. In some embodiments, the oral dosage form comprises about 1% by weight to about 10% by weight of the crystalline hydrochloride salt of the compound of Formula (I), in anhydrous or monohydrate form, based on the total weight of the oral dosage form. In some embodiments, the oral dosage form comprises about 10% by weight to about 20% by weight of the crystalline hydrochloride salt of the compound of Formula (I), in anhydrous or monohydrate form, based on the total weight of the oral dosage form. In some embodiments, the oral dosage form comprises about 10% by weight to about 15% by weight of the crystalline hydrochloride salt of the compound of Formula (I), in anhydrous or monohydrate form, based on the total weight of the oral dosage form. In some embodiments, the oral dosage form comprises about 1 mg to about 60 mg of the crystalline hydrochloride salt in anhydrous or monohydrate form. In some embodiments, the oral dosage form comprises about 10 mg to about 60 mg of the crystalline hydrochloride salt in anhydrous or monohydrate form. In some embodiments, the oral dosage form comprises the anhydrous form of the crystalline hydrochloride salt of the compound of Formula (I) in an amount of about 27.2 mg. In some embodiments, the oral dosage form comprises the anhydrous form of the crystalline hydrochloride salt of the compound of Formula (I) an amount of about 54.3 mg. In some embodiments, the oral dosage form comprises about 28.2 mg or about 56.5 mg of the monohydrate form of the crystalline hydrochloride salt of the compound of Formula (I).

In some embodiments,
the one or more disintegrants is selected from the group consisting of cross-linked polyvinylpyrrolidone, croscarmellose sodium, and sodium starch glycolate; and
the one or more lubricants is selected from the group consisting of sodium stearyl fumarate, stearic acid, talc, calcium stearate, magnesium stearate, solid polyethylene glycols, and sodium lauryl sulfate. In some embodiments, the oral dosage form is a tablet or a capsule. In some embodiments, the oral dosage form is a capsule.

In an embodiment, provided herein is a pharmaceutically acceptable capsule comprising a compound of Formula (I):

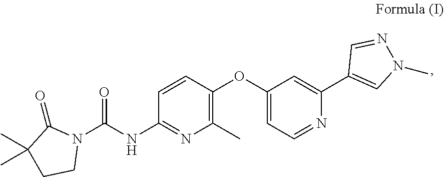

and one or more pharmaceutically acceptable excipients, wherein the compound is present in the pharmaceutically acceptable capsule as a free base, or a pharmaceutically acceptable salt of the compound and wherein the free base or pharmaceutically acceptable salt is in anhydrous or hydrate form.

In some embodiments, the compound of Formula (I) is present in the pharmaceutically acceptable capsule as a pharmaceutically acceptable salt in anhydrous or monohydrate form. In some embodiments, the compound of Formula (I) is present in the pharmaceutically acceptable capsule as an anhydrous form.

In some embodiments, the one or more pharmaceutically acceptable excipients is a filler. In some embodiments, the one or more pharmaceutically acceptable excipients is lactose monohydrate. In some embodiments, the pharmaceutically acceptable capsule comprises one or more disintegrants, one or more lubricants, or a combination thereof. In some embodiments, the pharmaceutically acceptable capsule further comprises one or more disintegrants. In some embodiments, the pharmaceutically acceptable capsule further comprises one or more lubricants.

In one embodiment, provided herein is a pharmaceutically acceptable capsule comprising:
about 1% by weight to about 70% by weight, based on the total weight of the pharmaceutically acceptable capsule, of a compound of Formula (I):

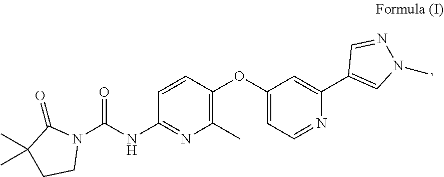

wherein the compound is present in the pharmaceutically acceptable capsule as a free base, a pharmaceutically acceptable salt of the compound and wherein the free base or pharmaceutically acceptable salt is in anhydrous or hydrate form, and one or more pharmaceutically acceptable excipients, wherein the pharmaceutically acceptable excipients comprise:
(b) about 20% by weight to about 97% by weight of one or more fillers based on the total weight of the pharmaceutically acceptable capsule;
(c) about 1% by weight to about 20% by weight of one or more disintegrants based on the total weight of the pharmaceutically acceptable capsule; and
(d) about 0.1% by weight to about 10% by weight of one or more lubricants based on the total weight of the pharmaceutically acceptable capsule.

In some embodiments, the pharmaceutically acceptable capsule comprises:
(a) about 10% by weight to about 30% by weight of the compound of Formula (I) based on the total weight of the pharmaceutically acceptable capsule;
(b) about 70% by weight to about 90% by weight of one or more fillers based on the total weight of the pharmaceutically acceptable capsule;
(c) about 1% by weight to about 10% by weight of one or more disintegrants based on the total weight of the pharmaceutically acceptable capsule; and
(d) about 0.1% by weight to about 3% by weight of one or more lubricants based on the total weight of the pharmaceutically acceptable capsule.

In some embodiments, the one or more pharmaceutically acceptable excipients is one or more fillers selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc, mannitol, maltodextrin, confectioner's sugar, compressible sugar, sorbitol, starch and talc. In some embodiments, the one or more fillers is lactose monohydrate. In some embodiments, the one or more disintegrants is one or more disintegrants selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone. In some embodiments, the one or more lubricants is one or more lubricants selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate. In some embodiments, the one or more pharmaceutically acceptable excipients is lactose monohydrate and the pharmaceutically acceptable capsule further comprises cross-linked polyvinylpyrrolidone and magnesium stearate. In some embodiments, the pharmaceutically acceptable capsule further comprises cross-linked polyvinylpyrrolidone and magnesium stearate. In some embodiments, the pharmaceutically acceptable capsule is for oral administration. In some embodiments, the pharmaceutically acceptable capsule is a solid dosage form. In some embodiments, the pharmaceutically acceptable capsule comprises about 1% by weight to about 30% by weight of the compound of Formula (I) based on the total weight of the pharmaceutically acceptable capsule. In some embodiments, the pharmaceutically acceptable capsule comprises about 1% by weight to about 10% by weight of the compound of Formula (I) based on the total weight of the pharmaceutically acceptable capsule. In some embodiments, the pharmaceutically acceptable capsule comprises about 10% by weight to about 20% by weight of the compound of Formula (I) based on the total weight of the pharmaceutically acceptable capsule. In some embodiments, the pharmaceutically acceptable capsule comprises about 10% by weight to about 15% by weight of the compound of Formula (I) based on the total weight of the pharmaceutically acceptable capsule. In some embodiments, the pharmaceutically acceptable capsule comprises about 1 mg to about 60 mg of the compound of Formula (I). In some embodiments, the compound of Formula (I) is present in the pharmaceutically acceptable capsule as a crystalline hydrochloride salt in anhydrous or monohydrate form. In some embodiments, the pharmaceutically acceptable capsule comprises about 27.2 mg or about 54.3 mg of the anhydrous form of the crystalline hydrochloride salt of the compound of Formula (I). In some embodiments, the pharmaceutically acceptable capsule comprises about 28.2 mg or about 56.5 mg of the monohydrate form of the crystalline hydrochloride salt of the compound of Formula (I). In some embodiments, the pharmaceutically acceptable capsule comprises the anhydrous form of the crystalline hydrochloride salt of the compound of Formula (I) in an amount sufficient to provide 25 mg of the compound of Formula (I). In some embodiments, the pharmaceutically acceptable capsule comprises the anhydrous form of the crystalline hydrochloride salt of the compound of Formula (I) in an amount sufficient to provide 50 mg of the compound of Formula (I). In some embodiments, the pharmaceutically acceptable capsule comprises the monohydrate form of the crystalline hydrochloride salt of the compound of Formula (I) in an amount sufficient to provide 25 mg of the compound of Formula (I). In some embodiments, the pharmaceutically acceptable capsule comprises the monohydrate form of the crystalline hydrochloride salt of the compound of Formula (I) in an amount sufficient to provide 50 mg of the compound of Formula (I).

In an embodiment, described herein is a pharmaceutically acceptable capsule comprising a crystalline hydrochloride of a compound of Formula (I):

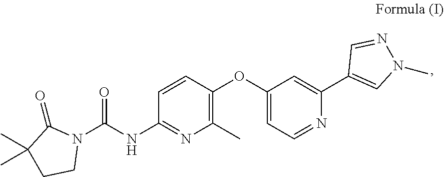

in anhydrous or hydrate form, and one or more pharmaceutically acceptable excipients. In some embodiments, the pharmaceutically acceptable capsule has not more than about 5 mol %, not more than about 3 mol %, or not more than about 1 mol % of other solid-state forms of the compound represented by Formula (I).

In some embodiments, the crystalline hydrochloride salt of the compound of Formula (I), in monohydrate form, has an X-ray powder diffraction pattern comprising peaks, in terms of 2-theta, at about 9.5°, 10.60, and 19.7° as measured by CuKα radiation. In some embodiments, the crystalline hydrochloride salt of the compound of Formula (I), in monohydrate form, has an X-ray powder diffraction pattern comprising peaks, in terms of 2-theta, at about 9.5°, 10.6°, 16.6°, 18.80, and 19.7° as measured by CuKα radiation. In some embodiments, the one or more pharmaceutically acceptable excipients is a filler. In some embodiments, the one or more pharmaceutically acceptable excipients is lactose monohydrate. In some embodiments, the pharmaceutically acceptable capsule further comprises one or more disintegrants, one or more lubricants, or a combination thereof. In some embodiments, the pharmaceutically acceptable capsule further comprises one or more disintegrants. In some embodiments, the pharmaceutically acceptable capsule further comprises one or more lubricants.

In an embodiment, described herein is a pharmaceutically acceptable capsule comprising:
(a) about 1% by weight to about 70% by weight, based on the total weight of the pharmaceutically acceptable capsule, of a crystalline hydrochloride salt of a compound of Formula (I):

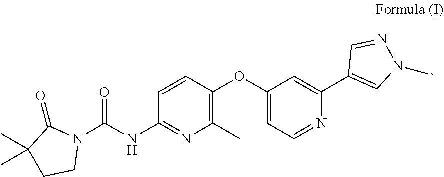

in anhydrous or monohydrate form, and one or more pharmaceutically acceptable excipients, wherein the one or more pharmaceutically acceptable excipients comprise:
(b) about 20% by weight to about 97% by weight of one or more fillers based on the total weight of the pharmaceutically acceptable capsule;
(c) about 1% by weight to about 20% by weight of one or more disintegrants based on the total weight of the pharmaceutically acceptable capsule; and
(d) about 0.1% by weight to about 10% by weight of one or more lubricants based on the total weight of the pharmaceutically acceptable capsule.

In some embodiments, the pharmaceutically acceptable capsule comprises:
(a) about 10% by weight to about 30% by weight of the crystalline hydrochloride salt of the compound of Formula (I) based on the total weight of the pharmaceutically acceptable capsule;
(b) about 70% by weight to about 90% by weight of one or more fillers based on the total weight of the pharmaceutically acceptable capsule;
(c) about 1% by weight to about 10% by weight of one or more disintegrants based on the total weight of the pharmaceutically acceptable capsule; and
(d) about 0.1% by weight to about 3% by weight of one or more lubricants based on the total weight of the pharmaceutically acceptable capsule.

In some embodiments, the pharmaceutically acceptable capsule has not having more than about 5, not more than about 3, or not more than about 1 mol % of other solid-state forms of the compound represented by Formula (I). In some embodiments, the one or more pharmaceutically acceptable excipients is one or more fillers selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc. In some embodiments, the one or more fillers is lactose monohydrate. In some embodiments, the one or more disintegrants is one or more disintegrants selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone. In some embodiments, the one or more lubricants is one or more lubricants selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate. In some embodiments, the one or more pharmaceutically acceptable excipients is lactose monohydrate, and the pharmaceutically acceptable capsule further comprises cross-linked polyvinylpyrrolidone and magnesium stearate. In some embodiments, the pharmaceutically acceptable capsule further comprises cross-linked polyvinylpyrrolidone and magnesium stearate. In some embodiments, the pharmaceutically acceptable capsule is for oral administration. In some embodiments, the pharmaceutically acceptable capsule comprises about 1% by weight to about 30% by weight of the crystalline hydrochloride salt of the compound of Formula (I), in anhydrous or monohydrate form, based on the total weight of the pharmaceutically acceptable capsule. In some embodiments, the pharmaceutically acceptable capsule comprises about 1% by weight to about 10% by weight of the crystalline hydrochloride salt of the compound of Formula (I), in anhydrous or monohydrate form, based on the total weight of the pharmaceutically acceptable capsule. In some embodiments, the pharmaceutically acceptable capsule comprises about 10% by weight to about 20% by weight of the crystalline hydrochloride salt of the compound of Formula (I) in anhydrous or monohydrate form, based on the total weight of the pharmaceutically acceptable capsule. In some embodiments, the pharmaceutically acceptable capsule comprises about 10% by weight to about 15% by weight of the crystalline hydrochloride salt of the compound of Formula (I), in anhydrous or monohydrate form, based on the total weight of the pharmaceutically acceptable capsule. In some embodiments, the oral dosage form comprises about 27.2 mg or about 54.3 mg of the anhydrous form of the crystalline hydrochloride salt of the compound of Formula (I). In some embodiments, the oral dosage form comprises about 28.2 mg or about 56.5 mg of the monohydrate form of the crystalline hydrochloride salt of the compound of Formula (I). In some embodiments, the pharmaceutically acceptable capsule comprises the anhydrous form of the crystalline hydrochloride salt of the compound of Formula (I) in an amount sufficient to provide 25 mg of the compound of Formula (I). In some embodiments, the pharmaceutically acceptable capsule comprises the anhydrous form of the crystalline hydrochloride salt of the compound of Formula (I) in an amount sufficient to provide 50 mg of the compound of Formula (I). In some embodiments, the pharmaceutically acceptable capsule comprises the monohydrate form of the crystalline hydrochloride salt of the compound of Formula (I) in an amount sufficient to provide 25 mg of the compound of Formula (I). In some embodiments, the pharmaceutically acceptable capsule comprises the monohydrate form of the crystalline hydrochloride salt of the compound of Formula (I) in an amount sufficient to provide 50 mg of the compound of Formula (I).

In an embodiment provided is a process of preparing a pharmaceutically acceptable unit formulation, the pharmaceutically acceptable unit formulation comprising:
(a) about 1% by weight to about 70% by weight, based on the weight of the pharmaceutically acceptable unit formulation of a crystalline hydrochloride salt of a compound of Formula (I):

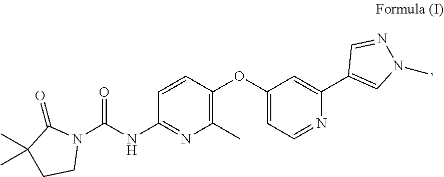

in anhydrous or monohydrate form, and one or more pharmaceutically acceptable excipients, wherein the one or more pharmaceutically acceptable excipients comprise:
(b) about 20% by weight to about 97% by weight of one or more fillers, based on the weight of the pharmaceutically acceptable unit formulation;
(c) about 1% by weight to about 20% by weight of one or more disintegrants, based on the weight of the pharmaceutically acceptable unit formulation; and
(d) about 0.1% by weight to about 10% by weight of one or more lubricants, based on the weight of the pharmaceutically acceptable unit formulation
   the process comprising:
(i) blending the crystalline hydrochloride salt of a compound represented by Formula (I) with the one or more fillers and one or more disintegrants to produce a first blend; and
(ii) blending the first blend with the one or more lubricants, thereby preparing the pharmaceutically acceptable unit formulation.

In some embodiments, the pharmaceutically acceptable unit formulation comprises:
(a) about 10% by weight to about 30% by weight of the compound of Formula (I) based on the weight of the pharmaceutically acceptable unit formulation;
(b) about 70% by weight to about 90% by weight of one or more fillers based on the weight of the pharmaceutically acceptable unit formulation;
(c) about 1% by weight to about 10% by weight of one or more disintegrants based on the weight of the pharmaceutically acceptable unit formulation; and
(d) about 0.1% by weight to about 3% by weight of one or more lubricants based on the weight of the pharmaceutically acceptable unit formulation.

In some embodiments, the pharmaceutically acceptable unit formulation has no more than about 5, no more than about 3, or no more than about 1 mol % of other solid-state forms of the compound represented by Formula (I). In some embodiments, the pharmaceutically acceptable unit formulation is for oral administration. In some embodiments, the pharmaceutically acceptable unit formulation is a solid dosage form.

In some embodiments, the pharmaceutically acceptable unit formulation is a tablet or a capsule. In some embodiments, the pharmaceutically acceptable unit formulation is a capsule. In some embodiments, the pharmaceutically acceptable unit formulation has an acceptance value of less than about 15 as determined by USP Uniformity of Dosage Unit <905>. In some embodiments, the oral dosage form comprises:
(a) about 1% by weight to about 70% by weight, based on the total weight of the oral dosage form, of a crystalline hydrochloride salt of a compound of Formula (I):

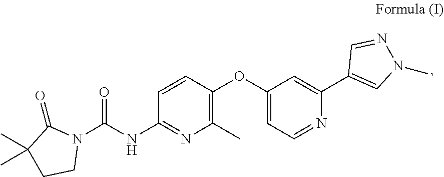

in anhydrous or monohydrate form, and one or more pharmaceutically acceptable excipients, wherein the one or more pharmaceutically acceptable excipients comprise:
(b) about 20% by weight to about 97% by weight of one or more fillers based on the total weight of the oral dosage form;
(c) about 1% by weight to about 20% by weight of one or more disintegrants based on the total weight of the oral dosage form; and
(d) about 0.1% by weight to about 10% by weight of one or more lubricants based on the total weight of the oral dosage form,
   the process comprising:
(i) blending the compound represented by Formula (I) with the one or more fillers and one or more disintegrants to produce a first blend; and
(ii) blending the first blend with the one or more lubricants, thereby preparing the oral dosage form.

In some embodiments, the oral dosage form comprises:
(a) about 10% by weight to about 30% by weight of the crystalline hydrochloride salt of the compound of Formula (I) based on the total weight of the oral dosage form;
(b) about 70% by weight to about 90% by weight of one or more fillers based on the total weight of the oral dosage form;
(c) about 1% by weight to about 10% by weight of one or more disintegrants based on the total weight of the oral dosage form; and
(d) about 0.1% by weight to about 3% by weight of one or more lubricants based on the total weight of the oral dosage form. In some embodiments, the oral dosage form has no more than about 5, no more than about 3, or no more than about 1 mol % of other solid-state forms of the compound represented by Formula (I). In some embodiments, the oral dosage form is a tablet or a capsule. In some embodiments, the oral dosage form is a capsule.

In one embodiment, provided is a process of preparing a pharmaceutically acceptable capsule, the pharmaceutically acceptable capsule comprising:
(a) about 1% to about 70%, based on the total weight of the pharmaceutically acceptable capsule, of a crystalline hydrochloride salt of a compound of Formula (I):

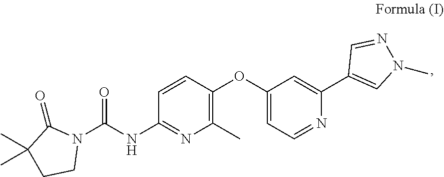

in anhydrous or monohydrate form, and one or more pharmaceutically acceptable excipients, wherein the one or more pharmaceutically acceptable excipients comprise:
(b) about 20% by weight to about 97% by weight of one or more fillers, based on the total weight of the pharmaceutically acceptable capsule;
(c) about 1% by weight to about 20% by weight of one or more disintegrants, based on the total weight of the pharmaceutically acceptable capsule; and
(d) about 0.1% by weight to about 10% by weight of one or more lubricants, based on the total weight of the pharmaceutically acceptable capsule;
   the process comprising:
(i) blending the compound represented by Formula (I) with the one or more fillers and one or more disintegrants to produce a first blend;
(ii) blending the first blend with the one or more lubricants to form a second blend; and
(iii) dispensing the second blend into an empty pharmaceutically acceptable capsule,
thereby forming the pharmaceutically acceptable capsule.

In some embodiments, the pharmaceutically acceptable capsule comprises:
(a) about 10% by weight to about 30% by weight of the compound of Formula (I) based on the total weight of the pharmaceutically acceptable capsule;
(b) about 70% by weight to about 90% by weight of one or more fillers based on the total weight of the pharmaceutically acceptable capsule;
(c) about 1% by weight to about 10% by weight of one or more disintegrants based on the total weight of the pharmaceutically acceptable capsule; and
(d) about 0.1% by weight to about 3% by weight of one or more lubricants based on the total weight of the pharmaceutically acceptable capsule. In some embodiments, the pharmaceutically acceptable capsule has no more than about 5, no more than about 3, or no more than about 1 mol % of other solid-state forms of the compound represented by Formula (I). In some embodiments, the pharmaceutically acceptable capsule is for oral administration. In some embodiments, the pharmaceutically acceptable capsule is a solid dosage form. In some embodiments, the one or more fillers is selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc, mannitol, maltodextrin, confectioner's sugar, compressible sugar, sorbitol, starch and talc. In some embodiments, the one or more fillers is lactose monohydrate. In some embodiments, the one or more disintegrants is selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides. In some embodiments, the one or more disintegrants is cross-linked polyvinylpyrrolidone. In some embodiments, the one or more lubricants is selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate. In some embodiments, the one or more lubricants is magnesium stearate. In some embodiments, the one or more fillers is lactose monohydrate, the one or more disintegrants is cross-linked polyvinylpyrrolidone and the one or more lubricants is magnesium stearate. In some embodiments, the pharmaceutically acceptable unit formulation, oral dosage form, or pharmaceutically acceptable capsule comprises about 1% by weight to about 30% by weight of the compound of Formula (I) based on the weight of the pharmaceutically acceptable unit formulation, oral dosage form, or pharmaceutically acceptable capsule. In some embodiments, the pharmaceutically acceptable unit formulation, oral dosage form, or pharmaceutically acceptable capsule comprises about 1% by weight to about 10% by weight of the compound of Formula (I) based on the weight of the pharmaceutically acceptable unit formulation, oral dosage form, or pharmaceutically acceptable capsule. In some embodiments, the pharmaceutically acceptable unit formulation, oral dosage form, or pharmaceutically acceptable capsule comprises about 10% by weight to about 20% by weight of the compound of Formula (I) based on the weight of the pharmaceutically acceptable unit formulation, oral dosage form, or pharmaceutically acceptable capsule. In some embodiments, the pharmaceutically acceptable unit formulation, oral dosage form, or pharmaceutically acceptable capsule comprises about 10% by weight to about 15% by weight of the compound of Formula (I) based on the weight of the pharmaceutically acceptable unit formulation, oral dosage form, or pharmaceutically acceptable capsule. In some embodiments, the pharmaceutically acceptable unit formulation, oral dosage form, or pharmaceutically acceptable capsule comprises the crystalline hydrochloride salt of the compound of Formula (I), in anhydrous or monohydrate form, in an amount sufficient to provide about 1 mg to about 60 mg of the compound of Formula (I). In some embodiments, the pharmaceutically acceptable unit formulation, oral dosage form, or pharmaceutically acceptable capsule the crystalline hydrochloride salt of the compound of Formula (I), in anhydrous or monohydrate form, in an amount sufficient to provide 25 mg or 50 mg of the compound of Formula (I).

In an embodiment, provided herein is a pharmaceutically acceptable unit formulation comprising:
(a) about 1% to about 70%, based on the weight of the pharmaceutically acceptable unit formulation, of a crystalline hydrochloride salt of a compound of Formula (I):

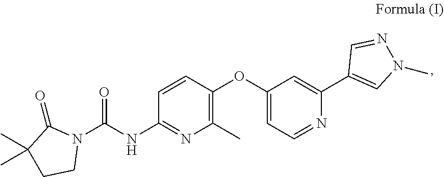

in anhydrous or monohydrate form, and one or more pharmaceutically acceptable excipients, wherein the one or more pharmaceutically acceptable excipients comprise:
(b) about 20% by weight to about 97% by weight of one or more fillers, based on the weight of the pharmaceutically acceptable unit formulation;
(c) about 1% by weight to about 20% by weight of one or more disintegrants, based on the weight of the pharmaceutically acceptable unit formulation; and
(d) about 0.1% by weight to about 10% by weight of one or more lubricants, based on the weight of the pharmaceutically acceptable unit formulation;
   made by a process comprising the steps of:
(i) blending the crystalline hydrochloride salt of compound represented by Formula (I) with the one or more fillers and one or more disintegrants to produce a first blend; and
(ii) blending the first blend with the one or more lubricants, thereby forming the pharmaceutically acceptable unit formulation.

In some embodiments, the pharmaceutically acceptable unit formulation has no more than about 5, no more than about 3, or no more than about 1 mol % of other solid-state forms of the compound represented by Formula (I).

In an embodiment, provided herein is an oral dosage form comprising:
(a) about 1% to about 70%, based on the total weight of the oral dosage form, of a crystalline hydrochloride salt of a compound of Formula (I):

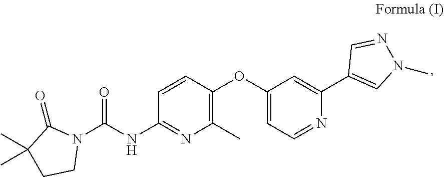

in anhydrous or monohydrate form, and one or more pharmaceutically acceptable excipients, wherein the one or more pharmaceutically acceptable excipients comprise:
(b) about 20% by weight to about 97% by weight of one or more fillers, based on the total weight of the oral dosage form;
(c) about 1% by weight to about 20% by weight of one or more disintegrants, based on the total weight of the oral dosage form; and
(d) about 0.1% by weight to about 10% by weight of one or more lubricants, based on the total weight of the oral dosage form;
   made by a process comprising the steps of:
(i) blending the crystalline hydrochloride salt of compound represented by Formula (I) with the one or more fillers and one or more disintegrants to produce a first blend; and
(ii) blending the first blend with the one or more lubricants,
thereby forming the oral dosage form.

In some embodiments, the oral dosage form has no more than about 5, no more than about 3, or no more than about 1 mol % of other solid-state forms of the compound represented by Formula (I).

In an embodiment, provided herein is a pharmaceutically acceptable capsule comprising:
(a) about 1% to about 70%, based on the total weight of the pharmaceutically acceptable capsule, of a crystalline hydrochloride salt of a compound of Formula (I):

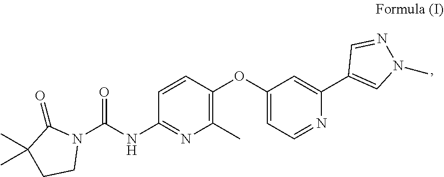

in anhydrous or monohydrate form, and one or more pharmaceutically acceptable excipients, wherein the one or more pharmaceutically acceptable excipients comprise:
(b) about 20% by weight to about 97% by weight of one or more fillers, based on the total weight of the pharmaceutically acceptable capsule;
(c) about 1% by weight to about 20% by weight of one or more disintegrants, based on the total weight of the pharmaceutically acceptable capsule; and
(d) about 0.1% by weight to about 10% by weight of one or more lubricants, based on the total weight of the pharmaceutically acceptable capsule;
   made by a process comprising the steps of:
(i) blending the crystalline hydrochloride salt of the compound represented by Formula (I) with the one or more fillers and one or more disintegrants to produce a first blend;
(ii) blending the first blend with the one or more lubricants to form a second blend; and
(iii) dispensing the second blend into an empty pharmaceutically acceptable capsule, thereby forming the pharmaceutically acceptable capsule.

In an embodiment, the pharmaceutically acceptable capsule has no more than about 5, no more than about 3, or no more than about 1 mol % of other solid-state forms of the compound represented by Formula (I).

Provided herein, in some embodiments, is a pharmaceutically acceptable formulation, comprising: a means for providing a compound of Formula (I):

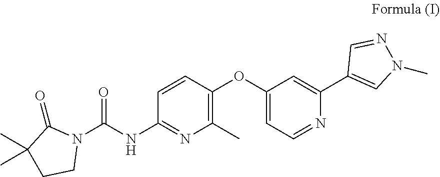

with a hygroscopic property appropriate for clinical preparation storage and/or for granulation, and a pharmaceutically acceptable excipient.

In some embodiments, the means for providing the compound of Formula (I) is a pharmaceutically acceptable salt form of the compound of Formula (I), wherein the salt form is in anhydrous or hydrate form. In some embodiments, the means for providing the compound of Formula (I) is a solid-state form of the pharmaceutically acceptable salt form of the compound of Formula (I).

Provided herein, in some embodiments, is a pharmaceutically acceptable capsule, comprising: a means for providing a compound of Formula (I):

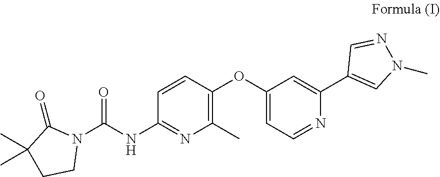

with a hygroscopic property appropriate for clinical preparation storage and/or for granulation, and a pharmaceutically acceptable excipient.

In some embodiments, the means for providing the compound of Formula (I) is a pharmaceutically acceptable salt form of the compound of Formula (I), wherein the salt form is in anhydrous or hydrate form. In some embodiments, the means for providing the compound of Formula (I) is a solid-state form of the pharmaceutically acceptable salt form of the compound of Formula (I).

### Methods of Use

Provided herein, in part, are methods for the treatment or prevention of a variety of diseases and disorders, which comprise administering to a patient in need of such treatment or prevention a therapeutically effective amount of one or more formulations (e.g., oral dosage formulations (e.g., capsules)) comprising a compound represented by Formula (I) as a free base, in its neutral form, in a solvate form, or in the form of a pharmaceutically acceptable salt; and one or more pharmaceutically acceptable excipients.

In some embodiments, provided herein is a formulation comprising the compound represented by Formula (I) which is for use in the treatment of a tenosynovial giant cell tumor (TGCT). In some embodiments, the tenosynovial giant cell tumor is a diffuse-type tenosynovial giant cell tumor. In some embodiments, the tenosynovial giant cell tumor is a localized tenosynovial giant cell tumor.

In some embodiments, provided herein is a formulation comprising the compound represented by Formula (I) which is for use in the treatment of graft versus host disease (GVHD). In some embodiments, the graft versus host disease (GVHD) is chronic graft versus host disease (cGVHD). In some embodiments, the graft versus host disease (GVHD) is acute graft versus host disease (aGVHD).

In some embodiments, provided herein is a formulation comprising the compound represented by Formula (I) which is for use in the treatment of a neurodegenerative disease. In some embodiments, the neurodegenerative disease is selected from the group consisting of Parkinson's disease (PD), Alzheimer's Disease (AD), frontotemporal dementia (FTD), amyotrophic lateral sclerosis (ALS), mild cognitive impairment, and Huntington's Disease (HD).

In some embodiments, provided herein is a formulation comprising the compound represented by Formula (I) which is for use in the treatment of a cancer selected from the group consisting of solid tumors, acute myeloid leukemia (AML), relapsed/refractory acute myeloid leukemia (AML), relapsed acute myeloid leukemia (AML), refractory acute myeloid leukemia (AML), myelodysplastic syndrome, acute lymphocytic leukemia (ALL), chronic lymphocytic leukemia (CLL), pancreatic ductal adenocarcinoma (PDAC), ovarian cancer, non-small cell lung cancer (NSCLC), prostate cancer, osteosarcoma, breast cancer, colon cancer, and glioblastoma. In some embodiments, the solid tumor is selected from the group consisting of ovarian cancer, pancreatic cancer, prostate cancer, lung cancer, breast cancer, kidney cancer, liver cancer, cervical cancer, bone metastatic cancer, papillary thyroid cancer, non-small cell lung cancer, colon cancer, and gastrointestinal stromal tumor.

In some embodiments, provided herein is a formulation comprising the compound represented by Formula (I) which is for use in the treatment of tumors known to have expression of colony-stimulating factor 1 receptor (CSF-1R) or its ligands, colony stimulating factor-1 (CSF-1), or interleukin (IL)-34 (IL-34).

In some embodiments, provided herein is a formulation comprising the compound represented by Formula (I) which is for use in the treatment of a disease or condition selected from the group consisting of metabolic diseases, rheumatoid arthritis, osteoarthritis, multiple sclerosis, autoimmune nephritis, lupus, Crohn's disease, asthma, chronic obstructive pulmonary diseases, osteoporosis, hypereosinophilic syndromes, mastocytosis, and histiocytosis.

In some embodiments, the formulation comprising the compound represented by Formula (I), can be useful in the treatment of diseases and conditions including, but not limited to, cancer, autoimmune diseases, and metabolic bone disorders, and other tumors related to the decreased proliferation, the depletion, or the repolarization of tumor-associated macrophages (TAMs) and treatment of associated disorders, for example but not limited to disorders disclosed herein such as tenosynovial giant cell tumor (TGCT), graft-versus-host disease (GVHD), or neurodegenerative diseases. In some embodiments, the treatment of the disease or conditions occurs through the inhibition of CSF-1R kinase.

In some embodiments, the formulation comprising the compound represented by Formula (I), can be useful in the inhibition of the proliferation of TAMs, the depletion of TAMs, the repolarization of protumoral M2 TAMs to antitumoral M1 type macrophages, and treatment of related disorders in patients, for example but not limited to disorders disclosed herein such as tenosynovial giant cell tumor (TGCT), graft-versus-host disease (GVHD), or neurodegenerative diseases. In some embodiments, the formulation comprising the compound represented by Formula (I), potently inhibit CSF-1R signaling. In some embodiments, the formulation comprising the compound represented by Formula (I), blocks macrophage-mediated tumor cell migration. In some embodiments, the formulation comprising the compound represented by Formula (I) blocks osteoclast differentiation. In some embodiments, formulation comprising the compound represented by Formula (I), blocks proliferation of a CSF-1R-dependent cell line. In some embodiments, the formulation comprising the compound represented by Formula (I), potently inhibit CSF-1R signaling in cellular assays, as well as blocks macrophage-mediated tumor cell migration, osteoclast differentiation, and proliferation of a CSF-1R-dependent cell line.

In some embodiments, the formulation comprising the compound represented by Formula (I), is selective in inhibiting CSF-1R over one or more of the FLT3, KIT, PDGFRα, PDGFRβ and VEGFR2 kinases. In some embodiments, the formulation comprising the compound represented by Formula (I), has greater than 100-fold selectivity in inhibiting CSF-1R over the FLT3, KIT, PDGFRα, PDGFRβ, and VEGFR2 kinases.

In some embodiments, the formulation comprising the compound represented by Formula (I), can be useful in the treatment of cancer. In some embodiments, such cancer may include glioblastoma (e.g., recurrent glioblastoma (GBM)), castrate resistant prostate cancer (CRPC), bone metastatic CRPC, cholangiocarcinoma (e.g., unresectable intrahepatic cholangiocarcinoma), ovarian cancer, pancreatic cancer, prostate cancer (e.g., advanced castration-resistant prostate cancer with bone metastasis and high circulating tumor cell counts), lung cancer, breast cancer, kidney cancer, liver cancer, cervical cancer, metastatic cancer (e.g., cancers that are metastatic to bone), papillary thyroid cancer, non-small cell lung cancer (NSCLC), colon cancer, colorectal cancer, gastrointestinal stromal tumor (GIST), solid tumors (e.g., refractory solid tumors, malignant solid tumors, metastatic breast or prostate cancer with bone disease, gastric, ovarian or non-small cell lung cancer that has malignant associated ascites or effusion(s)), advanced solid tumors (e.g., advanced incurable solid tumors in which the target kinases are linked to disease pathophysiology), melanoma, advanced melanoma, mesothelioma, multiple myeloma, follicular lymphoma, leukemia (e.g., refractory leukemia, acute myeloid leukemia (AML), relapsed/refractory acute myeloid leukemia (AML), relapsed acute myeloid leukemia (AML), refractory acute myeloid leukemia (AML), mast cell leukemia associated with CSF-1R), classic Hodgkin's lymphoma (cHL), relapsed or refractory cHL, peripheral T cell lymphoma, neurofibroma, sarcoma (e.g., soft tissue sarcoma, osteosarcoma, advanced sarcoma, high grade sarcoma, leiomyosarcoma, undifferentiated pleomorphic sarcoma, myxofibrosarcoma, dedifferentiated liposarcoma), refractory solid malignancies (e.g., colorectal, breast, pancreatic, prostate, NSCLC), endometrial, urothelial, salivary gland, trophoblastic tumor, gallbladder, renal cell carcinoma, chordoma, and gastric cancer, recurrent platinum-resistant epithelial ovarian, peritoneal, or fallopian tube cancer, squamous cell carcinoma of the head and neck, malignant peripheral nerve sheath tumors, hepatocellular carcinoma, and neoplasms (e.g., advanced malignant neoplasm, unresectable malignant neoplasm).

In some embodiments, the formulation comprising the compound represented by Formula (I), can be useful in the treatment of diseases and conditions including idiopathic pulmonary fibrosis (IPF), hyperproliferative diseases, metabolic diseases, myeloproliferative diseases, stroke, SARS-CoV2, hepatic inflammation, rheumatoid arthritis, osteoarthritis, multiple sclerosis, autoimmune nephritis, lupus (e.g., lupus nephritis, systemic lupus erythematosus (SLE), Crohn's disease, asthma, psoriasis, chronic obstructive pulmonary diseases, pulmonary arterial hypertension (PAH), osteoporosis, hypereosinophilic syndromes, neurofibromatosis type 1-associated plexiform neurofibromas and mastocytosis.

In some embodiments, provided herein is a method of treating a tenosynovial giant cell tumor, comprising administering to the patient a therapeutically effective amount of the compound represented by Formula (I), or a therapeutically effective amount of the formulation comprising the compound represented by Formula (I). In some embodiments, the tenosynovial giant cell tumor is a diffuse-type tenosynovial giant cell tumor. In some embodiments, the tenosynovial giant cell tumor is a localized tenosynovial giant cell tumor.

In some embodiments, such tenosynovial giant cell tumor may be localized, e.g., as a single, well-defined nodule. In some embodiments, such tenosynovial giant cell tumor may be diffuse-type tenosynovial giant cell tumor. In some embodiments, such tenosynovial giant cell tumor may be associated with benign tumors. In some embodiments, such tenosynovial giant cell tumor may be associated with multiple nodules which may be aggressive. In some embodiments, the method may include administering about 2 mg to about 150 mg of the compound of Formula (I) daily to a patient in need thereof. In some embodiments, the method may include administering about 10 mg to about 100 mg of the compound of Formula (I) daily to a patient in need thereof. In some embodiments, the method may include administering about 10 mg to about 60 mg of the compound of Formula (I) daily to a patient in need thereof. In some embodiments, the method may include administering about 10 mg to about 35 mg of the compound of Formula (I) daily to a patient in need thereof. In some embodiments, the method may include administering about 2 mg of the compound of Formula (I) daily to a patient in need thereof. In some embodiments, the method may include administering about 3 mg of the compound of Formula (I) daily to a patient in need thereof. In some embodiments, the method may include administering about 4 mg of the compound of Formula (I) daily to a patient in need thereof. In some embodiments, the method may include administering about 5 mg of the compound of Formula (I) daily to a patient in need thereof. In some embodiments, the method may include administering about 6 mg of the compound of Formula (I) daily to a patient in need thereof. In some embodiments, the method may include administering about 7 mg of the compound of Formula (I) daily to a patient in need thereof. In some embodiments, the method may include administering about 8 mg of the compound of Formula (I) daily to a patient in need thereof. In some embodiments, the method may include administering about 9 mg of the compound of Formula (I) daily to a patient in need thereof. In some embodiments, the method may include administering about 10 mg of the compound of Formula (I) daily to a patient in need thereof. In some embodiments, the method may include administering about 11 mg of the compound of Formula (I) daily to a patient in need thereof. In some embodiments, the method may include administering about 12 mg of the compound of Formula (I) daily to a patient in need thereof. In some embodiments, the method may include administering about 13 mg of the compound of Formula (I) daily to a patient in need thereof. In some embodiments, the method may include administering about 14 mg of the compound of Formula (I) daily to a patient in need thereof. In some embodiments, the method may include administering about 15 mg of the compound of Formula (I) daily to a patient in need thereof. In some embodiments, the method may include administering about 16 mg of the compound of Formula (I) daily to a patient in need thereof. In some embodiments, the method may include administering about 17 mg of the compound of Formula (I) daily to a patient in need thereof. In some embodiments, the method may include administering about 18 mg of the compound of Formula (I) daily to a patient in need thereof. In some embodiments, the method may include administering about 19 mg of the compound of Formula (I) daily to a patient in need thereof. In some embodiments, the method may include administering about 20 mg of the compound of Formula (I) daily to a patient in need thereof. In some embodiments, the method may include administering about 21 mg of the compound of Formula (I) daily to a patient in need thereof. In some embodiments, the method may include administering about 22 mg of the compound of Formula (I) daily to a patient in need thereof. In some embodiments, the method may include administering about 23 mg of the compound of Formula (I) daily to a patient in need thereof. In some embodiments, the method may include administering about 24 mg of the compound of Formula (I) daily to a patient in need thereof. In some embodiments, the method may include administering about 25 mg of the compound of Formula (I) daily to a patient in need thereof. In some embodiments, the method may include administering about 26 mg of the compound of Formula (I) daily to a patient in need thereof. In some embodiments, the method may include administering about 27 mg of the compound of Formula (I) daily to a patient in need thereof. In some embodiments, the method may include administering about 28 mg of the compound of Formula (I) daily to a patient in need thereof. In some embodiments, the method may include administering about 29 mg of the compound of Formula (I) daily to a patient in need thereof. In some embodiments, the method may include administering about 30 mg of the compound of Formula (I) daily to a patient in need thereof. In some embodiments, the method may include administering about 31 mg of the compound of Formula (I) daily to a patient in need thereof. In some embodiments, the method may include administering about 32 mg of the compound of Formula (I) daily to a patient in need thereof. In some embodiments, the method may include administering about 33 mg of the compound of Formula (I) daily to a patient in need thereof. In some embodiments, the method may include administering about 34 mg of the compound of Formula (I) daily to a patient in need thereof. In some embodiments, the method may include administering about 35 mg of the compound of Formula (I) daily to a patient in need thereof. In some embodiments, the method may include administering about 36 mg of the compound of Formula (I) daily to a patient in need thereof. In some embodiments, the method may include administering about 36 mg of the compound of Formula (I) daily to a patient in need thereof. In some embodiments, the method may include administering about 37 mg of the compound of Formula (I) daily to a patient in need thereof. In some embodiments, the method may include administering about 38 mg of the compound of Formula (I) daily to a patient in need thereof. In some embodiments, the method may include administering about 39 mg of the compound of Formula (I) daily to a patient in need thereof. In some embodiments, the method may include administering about 40 mg of the compound of Formula (I) daily to a patient in need thereof. In some embodiments, the method may include administering about 41 mg of the compound of Formula (I) daily to a patient in need thereof. In some embodiments, the method may include administering about 42 mg of the compound of Formula (I) daily to a patient in need thereof. In some embodiments, the method may include administering about 43 mg of the compound of Formula (I) daily to a patient in need thereof. In some embodiments, the method may include administering about 44 mg of the compound of Formula (I) daily to a patient in need thereof. In some embodiments, the method may include administering about 45 mg of the compound of Formula (I) daily to a patient in need thereof. In some embodiments, the method may include administering about 46 mg of the compound of Formula (I) daily to a patient in need thereof. In some embodiments, the method may include administering about 47 mg of the compound of Formula (I) daily to a patient in need thereof. In some embodiments, the method may include administering about 48 mg of the compound of Formula (I) daily to a patient in need thereof. In some embodiments, the method may include administering about 49 mg of the compound of Formula (I) daily to a patient in need thereof. In some embodiments, the method may include administering about 50 mg of the compound of Formula (I) daily to a patient in need thereof. In some embodiments, the method may include administering about 51 mg of the compound of Formula (I) daily to a patient in need thereof. In some embodiments, the method may include administering about 52 mg of the compound of Formula (I) daily to a patient in need thereof. In some embodiments, the method may include administering about 53 mg of the compound of Formula (I) daily to a patient in need thereof. In some embodiments, the method may include administering about 54 mg of the compound of Formula (I) daily to a patient in need thereof. In some embodiments, the method may include administering about 55 mg of the compound of Formula (I) daily to a patient in need thereof. In some embodiments, the method may include administering about 56 mg of the compound of Formula (I) daily to a patient in need thereof. In some embodiments, the method may include administering about 57 mg of the compound of Formula (I) daily to a patient in need thereof. In some embodiments, the method may include administering about 58 mg of the compound of Formula (I) daily to a patient in need thereof. In some embodiments, the method may include administering about 59 mg of the compound of Formula (I) daily to a patient in need thereof. In some embodiments, the method may include administering about 60 mg of the compound of Formula (I) daily to a patient in need thereof. In some embodiments, the method may include administering about 65 mg of the compound of Formula (I) daily to a patient in need thereof. In some embodiments, the method may include administering about 70 mg of the compound of Formula (I) daily to a patient in need thereof. In some embodiments, the method may include administering about 75 mg of the compound of Formula (I) daily to a patient in need thereof. In some embodiments, the method may include administering about 80 mg of the compound of Formula (I) daily to a patient in need thereof. In some embodiments, the method may include administering about 85 mg of the compound of Formula (I) daily to a patient in need thereof. In some embodiments, the method may include administering about 90 mg of the compound of Formula (I) daily to a patient in need thereof. In some embodiments, the method may include administering about 95 mg of the compound of Formula (I) daily to a patient in need thereof. In some embodiments, the method may include administering about 100 mg of the compound of Formula (I) daily to a patient in need thereof. In some embodiments, the method may include administering about 105 mg of the compound of Formula (I) daily to a patient in need thereof. In some embodiments, the method may include administering about 110 mg of the compound of Formula (I) daily to a patient in need thereof. In some embodiments, the method may include administering about 115 mg of the compound of Formula (I) daily to a patient in need thereof. In some embodiments, the method may include administering about 120 mg of the compound of Formula (I) daily to a patient in need thereof. In some embodiments, the method may include administering about 125 mg of the compound of Formula (I) daily to a patient in need thereof. In some embodiments, the method may include administering about 130 mg of the compound of Formula (I) daily to a patient in need thereof. In some embodiments, the method may include administering about 135 mg of the compound of Formula (I) daily to a patient in need thereof. In some embodiments, the method may include administering about 140 mg of the compound of Formula (I) daily to a patient in need thereof. In some embodiments, the method may include administering about 145 mg of the compound of Formula (I) daily to a patient in need thereof. In some embodiments, the method may include administering about 150 mg of the compound of Formula (I) daily to a patient in need thereof.

In some embodiments, the method may include administering about 2 mg to about 150 mg of the compound of Formula (I) once a week to a patient in need thereof. In some embodiments, the method may include administering about 10 mg to about 100 mg of the compound of Formula (I) once a week to a patient in need thereof. In some embodiments, the method may include administering about 10 mg to about 60 mg of the compound of Formula (I) once a week to a patient in need thereof. In some embodiments, the method may include administering about 10 mg to about 35 mg of the compound of Formula (I) once a week to a patient in need thereof. In some embodiments, the method may include administering about 2 mg of the compound of Formula (I) once a week to a patient in need thereof. In some embodiments, the method may include administering about 3 mg of the compound of Formula (I) once a week to a patient in need thereof. In some embodiments, the method may include administering about 4 mg of the compound of Formula (I) once a week to a patient in need thereof. In some embodiments, the method may include administering about 5 mg of the compound of Formula (I) once a week to a patient in need thereof. In some embodiments, the method may include administering about 6 mg of the compound of Formula (I) once a week to a patient in need thereof. In some embodiments, the method may include administering about 7 mg of the compound of Formula (I) once a week to a patient in need thereof. In some embodiments, the method may include administering about 8 mg of the compound of Formula (I) once a week to a patient in need thereof. In some embodiments, the method may include administering about 9 mg of the compound of Formula (I) once a week to a patient in need thereof. In some embodiments, the method may include administering about 10 mg of the compound of Formula (I) once a week to a patient in need thereof. In some embodiments, the method may include administering about 11 mg of the compound of Formula (I) once a week to a patient in need thereof. In some embodiments, the method may include administering about 12 mg of the compound of Formula (I) once a week to a patient in need thereof. In some embodiments, the method may include administering about 13 mg of the compound of Formula (I) once a week to a patient in need thereof. In some embodiments, the method may include administering about 14 mg of the compound of Formula (I) once a week to a patient in need thereof. In some embodiments, the method may include administering about 15 mg of the compound of Formula (I) once a week to a patient in need thereof. In some embodiments, the method may include administering about 16 mg of the compound of Formula (I) once a week to a patient in need thereof. In some embodiments, the method may include administering about 17 mg of the compound of Formula (I) once a week to a patient in need thereof. In some embodiments, the method may include administering about 18 mg of the compound of Formula (I) once a week to a patient in need thereof. In some embodiments, the method may include administering about 19 mg of the compound of Formula (I) once a week to a patient in need thereof. In some embodiments, the method may include administering about 20 mg of the compound of Formula (I) once a week to a patient in need thereof. In some embodiments, the method may include administering about 21 mg of the compound of Formula (I) once a week to a patient in need thereof. In some embodiments, the method may include administering about 22 mg of the compound of Formula (I) once a week to a patient in need thereof. In some embodiments, the method may include administering about 23 mg of the compound of Formula (I) once a week to a patient in need thereof. In some embodiments, the method may include administering about 24 mg of the compound of Formula (I) once a week to a patient in need thereof. In some embodiments, the method may include administering about 25 mg of the compound of Formula (I) once a week to a patient in need thereof. In some embodiments, the method may include administering about 26 mg of the compound of Formula (I) once a week to a patient in need thereof. In some embodiments, the method may include administering about 27 mg of the compound of Formula (I) once a week to a patient in need thereof. In some embodiments, the method may include administering about 28 mg of the compound of Formula (I) once a week to a patient in need thereof. In some embodiments, the method may include administering about 29 mg of the compound of Formula (I) once a week to a patient in need thereof. In some embodiments, the method may include administering about 30 mg of the compound of Formula (I) once a week to a patient in need thereof. In some embodiments, the method may include administering about 31 mg of the compound of Formula (I) once a week to a patient in need thereof. In some embodiments, the method may include administering about 32 mg of the compound of Formula (I) once a week to a patient in need thereof. In some embodiments, the method may include administering about 33 mg of the compound of Formula (I) once a week to a patient in need thereof. In some embodiments, the method may include administering about 34 mg of the compound of Formula (I) once a week to a patient in need thereof. In some embodiments, the method may include administering about 35 mg of the compound of Formula (I) once a week to a patient in need thereof. In some embodiments, the method may include administering about 36 mg of the compound of Formula (I) once a week to a patient in need thereof. In some embodiments, the method may include administering about 36 mg of the compound of Formula (I) once a week to a patient in need thereof. In some embodiments, the method may include administering about 37 mg of the compound of Formula (I) once a week to a patient in need thereof. In some embodiments, the method may include administering about 38 mg of the compound of Formula (I) once a week to a patient in need thereof. In some embodiments, the method may include administering about 39 mg of the compound of Formula (I) once a week to a patient in need thereof. In some embodiments, the method may include administering about 40 mg of the compound of Formula (I) once a week to a patient in need thereof. In some embodiments, the method may include administering about 41 mg of the compound of Formula (I) once a week to a patient in need thereof. In some embodiments, the method may include administering about 42 mg of the compound of Formula (I) once a week to a patient in need thereof. In some embodiments, the method may include administering about 43 mg of the compound of Formula (I) once a week to a patient in need thereof. In some embodiments, the method may include administering about 44 mg of the compound of Formula (I) once a week to a patient in need thereof. In some embodiments, the method may include administering about 45 mg of the compound of Formula (I) once a week to a patient in need thereof. In some embodiments, the method may include administering about 46 mg of the compound of Formula (I) once a week to a patient in need thereof. In some embodiments, the method may include administering about 47 mg of the compound of Formula (I) once a week to a patient in need thereof. In some embodiments, the method may include administering about 48 mg of the compound of Formula (I) once a week to a patient in need thereof. In some embodiments, the method may include administering about 49 mg of the compound of Formula (I) once a week to a patient in need thereof. In some embodiments, the method may include administering about 50 mg of the compound of Formula (I) once a week to a patient in need thereof. In some embodiments, the method may include administering about 51 mg of the compound of Formula (I) once a week to a patient in need thereof. In some embodiments, the method may include administering about 52 mg of the compound of Formula (I) once a week to a patient in need thereof. In some embodiments, the method may include administering about 53 mg of the compound of Formula (I) once a week to a patient in need thereof. In some embodiments, the method may include administering about 54 mg of the compound of Formula (I) once a week to a patient in need thereof. In some embodiments, the method may include administering about 55 mg of the compound of Formula (I) once a week to a patient in need thereof. In some embodiments, the method may include administering about 56 mg of the compound of Formula (I) once a week to a patient in need thereof. In some embodiments, the method may include administering about 57 mg of the compound of Formula (I) once a week to a patient in need thereof. In some embodiments, the method may include administering about 58 mg of the compound of Formula (I) once a week to a patient in need thereof. In some embodiments, the method may include administering about 59 mg of the compound of Formula (I) once a week to a patient in need thereof. In some embodiments, the method may include administering about 60 mg of the compound of Formula (I) once a week to a patient in need thereof. In some embodiments, the method may include administering about 65 mg of the compound of Formula (I) once a week to a patient in need thereof. In some embodiments, the method may include administering about 70 mg of the compound of Formula (I) once a week to a patient in need thereof. In some embodiments, the method may include administering about 75 mg of the compound of Formula (I) once a week to a patient in need thereof. In some embodiments, the method may include administering about 80 mg of the compound of Formula (I) once a week to a patient in need thereof. In some embodiments, the method may include administering about 85 mg of the compound of Formula (I) once a week to a patient in need thereof. In some embodiments, the method may include administering about 90 mg of the compound of Formula (I) once a week to a patient in need thereof. In some embodiments, the method may include administering about 95 mg of the compound of Formula (I) once a week to a patient in need thereof. In some embodiments, the method may include administering about 100 mg of the compound of Formula (I) once a week to a patient in need thereof. In some embodiments, the method may include administering about 105 mg of the compound of Formula (I) once a week to a patient in need thereof. In some embodiments, the method may include administering about 110 mg of the compound of Formula (I) once a week to a patient in need thereof. In some embodiments, the method may include administering about 115 mg of the compound of Formula (I) once a week to a patient in need thereof. In some embodiments, the method may include administering about 120 mg of the compound of Formula (I) once a week to a patient in need thereof. In some embodiments, the method may include administering about 125 mg of the compound of Formula (I) once a week to a patient in need thereof. In some embodiments, the method may include administering about 130 mg of the compound of Formula (I) once a week to a patient in need thereof. In some embodiments, the method may include administering about 135 mg of the compound of Formula (I) once a week to a patient in need thereof. In some embodiments, the method may include administering about 140 mg of the compound of Formula (I) once a week to a patient in need thereof. In some embodiments, the method may include administering about 145 mg of the compound of Formula (I) once a week to a patient in need thereof. In some embodiments, the method may include administering about 150 mg of the compound of Formula (I) once a week to a patient in need thereof.

In some embodiments, the method may include administering about 2 mg to about 150 mg of the compound of Formula (I) twice a week to a patient in need thereof. In some embodiments, the method may include administering about 10 mg to about 100 mg of the compound of Formula (I) twice a week to a patient in need thereof. In some embodiments, the method may include administering about 10 mg to about 60 mg of the compound of Formula (I) twice a week to a patient in need thereof. In some embodiments, the method may include administering about 10 mg to about 35 mg of the compound of Formula (I) twice a week to a patient in need thereof. In some embodiments, the method may include administering about 2 mg of the compound of Formula (I) twice a week to a patient in need thereof. In some embodiments, the method may include administering about 3 mg of the compound of Formula (I) twice a week to a patient in need thereof. In some embodiments, the method may include administering about 4 mg of the compound of Formula (I) twice a week to a patient in need thereof. In some embodiments, the method may include administering about 5 mg of the compound of Formula (I) twice a week to a patient in need thereof. In some embodiments, the method may include administering about 6 mg of the compound of Formula (I) twice a week to a patient in need thereof. In some embodiments, the method may include administering about 7 mg of the compound of Formula (I) twice a week to a patient in need thereof. In some embodiments, the method may include administering about 8 mg of the compound of Formula (I) twice a week to a patient in need thereof. In some embodiments, the method may include administering about 9 mg of the compound of Formula (I) twice a week to a patient in need thereof. In some embodiments, the method may include administering about 10 mg of the compound of Formula (I) twice a week to a patient in need thereof. In some embodiments, the method may include administering about 11 mg of the compound of Formula (I) twice a week to a patient in need thereof. In some embodiments, the method may include administering about 12 mg of the compound of Formula (I) twice a week to a patient in need thereof. In some embodiments, the method may include administering about 13 mg of the compound of Formula (I) twice a week to a patient in need thereof. In some embodiments, the method may include administering about 14 mg of the compound of Formula (I) twice a week to a patient in need thereof. In some embodiments, the method may include administering about 15 mg of the compound of Formula (I) twice a week to a patient in need thereof. In some embodiments, the method may include administering about 16 mg of the compound of Formula (I) twice a week to a patient in need thereof. In some embodiments, the method may include administering about 17 mg of the compound of Formula (I) twice a week to a patient in need thereof. In some embodiments, the method may include administering about 18 mg of the compound of Formula (I) twice a week to a patient in need thereof. In some embodiments, the method may include administering about 19 mg of the compound of Formula (I) twice a week to a patient in need thereof. In some embodiments, the method may include administering about 20 mg of the compound of Formula (I) twice a week to a patient in need thereof. In some embodiments, the method may include administering about 21 mg of the compound of Formula (I) twice a week to a patient in need thereof. In some embodiments, the method may include administering about 22 mg of the compound of Formula (I) twice a week to a patient in need thereof. In some embodiments, the method may include administering about 23 mg of the compound of Formula (I) twice a week to a patient in need thereof. In some embodiments, the method may include administering about 24 mg of the compound of Formula (I) twice a week to a patient in need thereof. In some embodiments, the method may include administering about 25 mg of the compound of Formula (I) twice a week to a patient in need thereof. In some embodiments, the method may include administering about 26 mg of the compound of Formula (I) twice a week to a patient in need thereof. In some embodiments, the method may include administering about 27 mg of the compound of Formula (I) twice a week to a patient in need thereof. In some embodiments, the method may include administering about 28 mg of the compound of Formula (I) twice a week to a patient in need thereof. In some embodiments, the method may include administering about 29 mg of the compound of Formula (I) twice a week to a patient in need thereof. In some embodiments, the method may include administering about 30 mg of the compound of Formula (I) twice a week to a patient in need thereof. In some embodiments, the method may include administering about 31 mg of the compound of Formula (I) twice a week to a patient in need thereof. In some embodiments, the method may include administering about 32 mg of the compound of Formula (I) twice a week to a patient in need thereof. In some embodiments, the method may include administering about 33 mg of the compound of Formula (I) twice a week to a patient in need thereof. In some embodiments, the method may include administering about 34 mg of the compound of Formula (I) twice a week to a patient in need thereof. In some embodiments, the method may include administering about 35 mg of the compound of Formula (I) twice a week to a patient in need thereof. In some embodiments, the method may include administering about 36 mg of the compound of Formula (I) twice a week to a patient in need thereof. In some embodiments, the method may include administering about 36 mg of the compound of Formula (I) twice a week to a patient in need thereof. In some embodiments, the method may include administering about 37 mg of the compound of Formula (I) twice a week to a patient in need thereof. In some embodiments, the method may include administering about 38 mg of the compound of Formula (I) twice a week to a patient in need thereof. In some embodiments, the method may include administering about 39 mg of the compound of Formula (I) twice a week to a patient in need thereof. In some embodiments, the method may include administering about 40 mg of the compound of Formula (I) twice a week to a patient in need thereof. In some embodiments, the method may include administering about 41 mg of the compound of Formula (I) twice a week to a patient in need thereof. In some embodiments, the method may include administering about 42 mg of the compound of Formula (I) twice a week to a patient in need thereof. In some embodiments, the method may include administering about 43 mg of the compound of Formula (I) twice a week to a patient in need thereof. In some embodiments, the method may include administering about 44 mg of the compound of Formula (I) twice a week to a patient in need thereof. In some embodiments, the method may include administering about 45 mg of the compound of Formula (I) twice a week to a patient in need thereof. In some embodiments, the method may include administering about 46 mg of the compound of Formula (I) twice a week to a patient in need thereof. In some embodiments, the method may include administering about 47 mg of the compound of Formula (I) twice a week to a patient in need thereof. In some embodiments, the method may include administering about 48 mg of the compound of Formula (I) twice a week to a patient in need thereof. In some embodiments, the method may include administering about 49 mg of the compound of Formula (I) twice a week to a patient in need thereof. In some embodiments, the method may include administering about 50 mg of the compound of Formula (I) twice a week to a patient in need thereof. In some embodiments, the method may include administering about 51 mg of the compound of Formula (I) twice a week to a patient in need thereof. In some embodiments, the method may include administering about 52 mg of the compound of Formula (I) twice a week to a patient in need thereof. In some embodiments, the method may include administering about 53 mg of the compound of Formula (I) twice a week to a patient in need thereof. In some embodiments, the method may include administering about 54 mg of the compound of Formula (I) twice a week to a patient in need thereof. In some embodiments, the method may include administering about 55 mg of the compound of Formula (I) twice a week to a patient in need thereof. In some embodiments, the method may include administering about 56 mg of the compound of Formula (I) twice a week to a patient in need thereof. In some embodiments, the method may include administering about 57 mg of the compound of Formula (I) twice a week to a patient in need thereof. In some embodiments, the method may include administering about 58 mg of the compound of Formula (I) twice a week to a patient in need thereof. In some embodiments, the method may include administering about 59 mg of the compound of Formula (I) twice a week to a patient in need thereof. In some embodiments, the method may include administering about 60 mg of the compound of Formula (I) twice a week to a patient in need thereof. In some embodiments, the method may include administering about 65 mg of the compound of Formula (I) twice a week to a patient in need thereof. In some embodiments, the method may include administering about 70 mg of the compound of Formula (I) twice a week to a patient in need thereof. In some embodiments, the method may include administering about 75 mg of the compound of Formula (I) twice a week to a patient in need thereof. In some embodiments, the method may include administering about 80 mg of the compound of Formula (I) twice a week to a patient in need thereof. In some embodiments, the method may include administering about 85 mg of the compound of Formula (I) twice a week to a patient in need thereof. In some embodiments, the method may include administering about 90 mg of the compound of Formula (I) twice a week to a patient in need thereof. In some embodiments, the method may include administering about 95 mg of the compound of Formula (I) twice a week to a patient in need thereof. In some embodiments, the method may include administering about 100 mg of the compound of Formula (I) twice a week to a patient in need thereof. In some embodiments, the method may include administering about 105 mg of the compound of Formula (I) twice a week to a patient in need thereof. In some embodiments, the method may include administering about 110 mg of the compound of Formula (I) twice a week to a patient in need thereof. In some embodiments, the method may include administering about 115 mg of the compound of Formula (I) twice a week to a patient in need thereof. In some embodiments, the method may include administering about 120 mg of the compound of Formula (I) twice a week to a patient in need thereof. In some embodiments, the method may include administering about 125 mg of the compound of Formula (I) twice a week to a patient in need thereof. In some embodiments, the method may include administering about 130 mg of the compound of Formula (I) twice a week to a patient in need thereof. In some embodiments, the method may include administering about 135 mg of the compound of Formula (I) twice a week to a patient in need thereof. In some embodiments, the method may include administering about 140 mg of the compound of Formula (I) twice a week to a patient in need thereof. In some embodiments, the method may include administering about 145 mg of the compound of Formula (I) twice a week to a patient in need thereof. In some embodiments, the method may include administering about 150 mg of the compound of Formula (I) twice a week to a patient in need thereof.

In some embodiments, the method may include administering about 2 mg to about 150 mg of the compound of Formula (I) once daily to a patient in need thereof. In some embodiments, the method may include administering about 10 mg to about 100 mg of the compound of Formula (I) once daily to a patient in need thereof. In some embodiments, the method may include administering about 10 mg to about 60 mg of the compound of Formula (I) once daily to a patient in need thereof. In some embodiments, the method may include administering about 10 mg to about 35 mg of the compound of Formula (I) once daily to a patient in need thereof. In some embodiments, the method may include administering about 2 mg of the compound of Formula (I) once daily to a patient in need thereof. In some embodiments, the method may include administering about 3 mg of the compound of Formula (I) once daily to a patient in need thereof. In some embodiments, the method may include administering about 4 mg of the compound of Formula (I) once daily to a patient in need thereof. In some embodiments, the method may include administering about 5 mg of the compound of Formula (I) once daily to a patient in need thereof. In some embodiments, the method may include administering about 6 mg of the compound of Formula (I) once daily to a patient in need thereof. In some embodiments, the method may include administering about 7 mg of the compound of Formula (I) once daily to a patient in need thereof. In some embodiments, the method may include administering about 8 mg of the compound of Formula (I) once daily to a patient in need thereof. In some embodiments, the method may include administering about 9 mg of the compound of Formula (I) once daily to a patient in need thereof. In some embodiments, the method may include administering about 10 mg of the compound of Formula (I) once daily to a patient in need thereof. In some embodiments, the method may include administering about 11 mg of the compound of Formula (I) once daily to a patient in need thereof. In some embodiments, the method may include administering about 12 mg of the compound of Formula (I) once daily to a patient in need thereof. In some embodiments, the method may include administering about 13 mg of the compound of Formula (I) once daily to a patient in need thereof. In some embodiments, the method may include administering about 14 mg of the compound of Formula (I) once daily to a patient in need thereof. In some embodiments, the method may include administering about 15 mg of the compound of Formula (I) once daily to a patient in need thereof. In some embodiments, the method may include administering about 16 mg of the compound of Formula (I) once daily to a patient in need thereof. In some embodiments, the method may include administering about 17 mg of the compound of Formula (I) once daily to a patient in need thereof. In some embodiments, the method may include administering about 18 mg of the compound of Formula (I) once daily to a patient in need thereof. In some embodiments, the method may include administering about 19 mg of the compound of Formula (I) once daily to a patient in need thereof. In some embodiments, the method may include administering about 20 mg of the compound of Formula (I) once daily to a patient in need thereof. In some embodiments, the method may include administering about 21 mg of the compound of Formula (I) once daily to a patient in need thereof. In some embodiments, the method may include administering about 22 mg of the compound of Formula (I) once daily to a patient in need thereof. In some embodiments, the method may include administering about 23 mg of the compound of Formula (I) once daily to a patient in need thereof. In some embodiments, the method may include administering about 24 mg of the compound of Formula (I) once daily to a patient in need thereof. In some embodiments, the method may include administering about 25 mg of the compound of Formula (I) once daily to a patient in need thereof. In some embodiments, the method may include administering about 26 mg of the compound of Formula (I) once daily to a patient in need thereof. In some embodiments, the method may include administering about 27 mg of the compound of Formula (I) once daily to a patient in need thereof. In some embodiments, the method may include administering about 28 mg of the compound of Formula (I) once daily to a patient in need thereof. In some embodiments, the method may include administering about 29 mg of the compound of Formula (I) once daily to a patient in need thereof. In some embodiments, the method may include administering about 30 mg of the compound of Formula (I) once daily to a patient in need thereof. In some embodiments, the method may include administering about 31 mg of the compound of Formula (I) once daily to a patient in need thereof. In some embodiments, the method may include administering about 32 mg of the compound of Formula (I) once daily to a patient in need thereof. In some embodiments, the method may include administering about 33 mg of the compound of Formula (I) once daily to a patient in need thereof. In some embodiments, the method may include administering about 34 mg of the compound of Formula (I) once daily to a patient in need thereof. In some embodiments, the method may include administering about 35 mg of the compound of Formula (I) once daily to a patient in need thereof. In some embodiments, the method may include administering about 36 mg of the compound of Formula (I) once daily to a patient in need thereof. In some embodiments, the method may include administering about 36 mg of the compound of Formula (I) once daily to a patient in need thereof. In some embodiments, the method may include administering about 37 mg of the compound of Formula (I) once daily to a patient in need thereof. In some embodiments, the method may include administering about 38 mg of the compound of Formula (I) once daily to a patient in need thereof. In some embodiments, the method may include administering about 39 mg of the compound of Formula (I) once daily to a patient in need thereof. In some embodiments, the method may include administering about 40 mg of the compound of Formula (I) once daily to a patient in need thereof. In some embodiments, the method may include administering about 41 mg of the compound of Formula (I) once daily to a patient in need thereof. In some embodiments, the method may include administering about 42 mg of the compound of Formula (I) once daily to a patient in need thereof. In some embodiments, the method may include administering about 43 mg of the compound of Formula (I) once daily to a patient in need thereof. In some embodiments, the method may include administering about 44 mg of the compound of Formula (I) once daily to a patient in need thereof. In some embodiments, the method may include administering about 45 mg of the compound of Formula (I) once daily to a patient in need thereof. In some embodiments, the method may include administering about 46 mg of the compound of Formula (I) once daily to a patient in need thereof. In some embodiments, the method may include administering about 47 mg of the compound of Formula (I) once daily to a patient in need thereof. In some embodiments, the method may include administering about 48 mg of the compound of Formula (I) once daily to a patient in need thereof. In some embodiments, the method may include administering about 49 mg of the compound of Formula (I) once daily to a patient in need thereof. In some embodiments, the method may include administering about 50 mg of the compound of Formula (I) once daily to a patient in need thereof. In some embodiments, the method may include administering about 51 mg of the compound of Formula (I) once daily to a patient in need thereof. In some embodiments, the method may include administering about 52 mg of the compound of Formula (I) once daily to a patient in need thereof. In some embodiments, the method may include administering about 53 mg of the compound of Formula (I) once daily to a patient in need thereof. In some embodiments, the method may include administering about 54 mg of the compound of Formula (I) once daily to a patient in need thereof. In some embodiments, the method may include administering about 55 mg of the compound of Formula (I) once daily to a patient in need thereof. In some embodiments, the method may include administering about 56 mg of the compound of Formula (I) once daily to a patient in need thereof. In some embodiments, the method may include administering about 57 mg of the compound of Formula (I) once daily to a patient in need thereof. In some embodiments, the method may include administering about 58 mg of the compound of Formula (I) once daily to a patient in need thereof. In some embodiments, the method may include administering about 59 mg of the compound of Formula (I) once daily to a patient in need thereof. In some embodiments, the method may include administering about 60 mg of the compound of Formula (I) once daily to a patient in need thereof. In some embodiments, the method may include administering about 65 mg of the compound of Formula (I) once daily to a patient in need thereof. In some embodiments, the method may include administering about 70 mg of the compound of Formula (I) once daily to a patient in need thereof. In some embodiments, the method may include administering about 75 mg of the compound of Formula (I) once daily to a patient in need thereof. In some embodiments, the method may include administering about 80 mg of the compound of Formula (I) once daily to a patient in need thereof. In some embodiments, the method may include administering about 85 mg of the compound of Formula (I) once daily to a patient in need thereof. In some embodiments, the method may include administering about 90 mg of the compound of Formula (I) once daily to a patient in need thereof. In some embodiments, the method may include administering about 95 mg of the compound of Formula (I) once daily to a patient in need thereof. In some embodiments, the method may include administering about 100 mg of the compound of Formula (I) once daily to a patient in need thereof. In some embodiments, the method may include administering about 105 mg of the compound of Formula (I) once daily to a patient in need thereof. In some embodiments, the method may include administering about 110 mg of the compound of Formula (I) once daily to a patient in need thereof. In some embodiments, the method may include administering about 115 mg of the compound of Formula (I) once daily to a patient in need thereof. In some embodiments, the method may include administering about 120 mg of the compound of Formula (I) once daily to a patient in need thereof. In some embodiments, the method may include administering about 125 mg of the compound of Formula (I) once daily to a patient in need thereof. In some embodiments, the method may include administering about 130 mg of the compound of Formula (I) once daily to a patient in need thereof. In some embodiments, the method may include administering about 135 mg of the compound of Formula (I) once daily to a patient in need thereof. In some embodiments, the method may include administering about 140 mg of the compound of Formula (I) once daily to a patient in need thereof. In some embodiments, the method may include administering about 145 mg of the compound of Formula (I) once daily to a patient in need thereof. In some embodiments, the method may include administering about 150 mg of the compound of Formula (I) once daily to a patient in need thereof.

In some embodiments, the method may include administering about 2 mg to about 150 mg of the compound of Formula (I) three times a week to a patient in need thereof. In some embodiments, the method may include administering about 10 mg to about 100 mg of the compound of Formula (I) three times a week to a patient in need thereof. In some embodiments, the method may include administering about 10 mg to about 60 mg of the compound of Formula (I) three times a week to a patient in need thereof. In some embodiments, the method may include administering about 10 mg to about 35 mg of the compound of Formula (I) three times a week to a patient in need thereof. In some embodiments, the method may include administering about 2 mg of the compound of Formula (I) three times a week to a patient in need thereof. In some embodiments, the method may include administering about 3 mg of the compound of Formula (I) three times a week to a patient in need thereof. In some embodiments, the method may include administering about 4 mg of the compound of Formula (I) three times a week to a patient in need thereof. In some embodiments, the method may include administering about 5 mg of the compound of Formula (I) three times a week to a patient in need thereof. In some embodiments, the method may include administering about 6 mg of the compound of Formula (I) three times a week to a patient in need thereof. In some embodiments, the method may include administering about 7 mg of the compound of Formula (I) three times a week to a patient in need thereof. In some embodiments, the method may include administering about 8 mg of the compound of Formula (I) three times a week to a patient in need thereof. In some embodiments, the method may include administering about 9 mg of the compound of Formula (I) three times a week to a patient in need thereof. In some embodiments, the method may include administering about 10 mg of the compound of Formula (I) three times a week to a patient in need thereof. In some embodiments, the method may include administering about 11 mg of the compound of Formula (I) three times a week to a patient in need thereof. In some embodiments, the method may include administering about 12 mg of the compound of Formula (I) three times a week to a patient in need thereof. In some embodiments, the method may include administering about 13 mg of the compound of Formula (I) three times a week to a patient in need thereof. In some embodiments, the method may include administering about 14 mg of the compound of Formula (I) three times a week to a patient in need thereof. In some embodiments, the method may include administering about 15 mg of the compound of Formula (I) three times a week to a patient in need thereof. In some embodiments, the method may include administering about 16 mg of the compound of Formula (I) three times a week to a patient in need thereof. In some embodiments, the method may include administering about 17 mg of the compound of Formula (I) three times a week to a patient in need thereof. In some embodiments, the method may include administering about 18 mg of the compound of Formula (I) three times a week to a patient in need thereof. In some embodiments, the method may include administering about 19 mg of the compound of Formula (I) three times a week to a patient in need thereof. In some embodiments, the method may include administering about 20 mg of the compound of Formula (I) three times a week to a patient in need thereof. In some embodiments, the method may include administering about 21 mg of the compound of Formula (I) three times a week to a patient in need thereof. In some embodiments, the method may include administering about 22 mg of the compound of Formula (I) three times a week to a patient in need thereof. In some embodiments, the method may include administering about 23 mg of the compound of Formula (I) three times a week to a patient in need thereof. In some embodiments, the method may include administering about 24 mg of the compound of Formula (I) three times a week to a patient in need thereof. In some embodiments, the method may include administering about 25 mg of the compound of Formula (I) three times a week to a patient in need thereof. In some embodiments, the method may include administering about 26 mg of the compound of Formula (I) three times a week to a patient in need thereof. In some embodiments, the method may include administering about 27 mg of the compound of Formula (I) three times a week to a patient in need thereof. In some embodiments, the method may include administering about 28 mg of the compound of Formula (I) three times a week to a patient in need thereof. In some embodiments, the method may include administering about 29 mg of the compound of Formula (I) three times a week to a patient in need thereof. In some embodiments, the method may include administering about 30 mg of the compound of Formula (I) three times a week to a patient in need thereof. In some embodiments, the method may include administering about 31 mg of the compound of Formula (I) three times a week to a patient in need thereof. In some embodiments, the method may include administering about 32 mg of the compound of Formula (I) three times a week to a patient in need thereof. In some embodiments, the method may include administering about 33 mg of the compound of Formula (I) three times a week to a patient in need thereof. In some embodiments, the method may include administering about 34 mg of the compound of Formula (I) three times a week to a patient in need thereof. In some embodiments, the method may include administering about 35 mg of the compound of Formula (I) three times a week to a patient in need thereof. In some embodiments, the method may include administering about 36 mg of the compound of Formula (I) three times a week to a patient in need thereof. In some embodiments, the method may include administering about 36 mg of the compound of Formula (I) three times a week to a patient in need thereof. In some embodiments, the method may include administering about 37 mg of the compound of Formula (I) three times a week to a patient in need thereof. In some embodiments, the method may include administering about 38 mg of the compound of Formula (I) three times a week to a patient in need thereof. In some embodiments, the method may include administering about 39 mg of the compound of Formula (I) three times a week to a patient in need thereof. In some embodiments, the method may include administering about 40 mg of the compound of Formula (I) three times a week to a patient in need thereof. In some embodiments, the method may include administering about 41 mg of the compound of Formula (I) three times a week to a patient in need thereof. In some embodiments, the method may include administering about 42 mg of the compound of Formula (I) three times a week to a patient in need thereof. In some embodiments, the method may include administering about 43 mg of the compound of Formula (I) three times a week to a patient in need thereof. In some embodiments, the method may include administering about 44 mg of the compound of Formula (I) three times a week to a patient in need thereof. In some embodiments, the method may include administering about 45 mg of the compound of Formula (I) three times a week to a patient in need thereof. In some embodiments, the method may include administering about 46 mg of the compound of Formula (I) three times a week to a patient in need thereof. In some embodiments, the method may include administering about 47 mg of the compound of Formula (I) three times a week to a patient in need thereof. In some embodiments, the method may include administering about 48 mg of the compound of Formula (I) three times a week to a patient in need thereof. In some embodiments, the method may include administering about 49 mg of the compound of Formula (I) three times a week to a patient in need thereof. In some embodiments, the method may include administering about 50 mg of the compound of Formula (I) three times a week to a patient in need thereof. In some embodiments, the method may include administering about 51 mg of the compound of Formula (I) three times a week to a patient in need thereof. In some embodiments, the method may include administering about 52 mg of the compound of Formula (I) three times a week to a patient in need thereof. In some embodiments, the method may include administering about 53 mg of the compound of Formula (I) three times a week to a patient in need thereof. In some embodiments, the method may include administering about 54 mg of the compound of Formula (I) three times a week to a patient in need thereof. In some embodiments, the method may include administering about 55 mg of the compound of Formula (I) three times a week to a patient in need thereof. In some embodiments, the method may include administering about 56 mg of the compound of Formula (I) three times a week to a patient in need thereof. In some embodiments, the method may include administering about 57 mg of the compound of Formula (I) three times a week to a patient in need thereof. In some embodiments, the method may include administering about 58 mg of the compound of Formula (I) three times a week to a patient in need thereof. In some embodiments, the method may include administering about 59 mg of the compound of Formula (I) three times a week to a patient in need thereof. In some embodiments, the method may include administering about 60 mg of the compound of Formula (I) three times a week to a patient in need thereof. In some embodiments, the method may include administering about 65 mg of the compound of Formula (I) three times a week to a patient in need thereof. In some embodiments, the method may include administering about 70 mg of the compound of Formula (I) three times a week to a patient in need thereof. In some embodiments, the method may include administering about 75 mg of the compound of Formula (I) three times a week to a patient in need thereof. In some embodiments, the method may include administering about 80 mg of the compound of Formula (I) three times a week to a patient in need thereof. In some embodiments, the method may include administering about 85 mg of the compound of Formula (I) three times a week to a patient in need thereof. In some embodiments, the method may include administering about 90 mg of the compound of Formula (I) three times a week to a patient in need thereof. In some embodiments, the method may include administering about 95 mg of the compound of Formula (I) three times a week to a patient in need thereof. In some embodiments, the method may include administering about 100 mg of the compound of Formula (I) three times a week to a patient in need thereof. In some embodiments, the method may include administering about 105 mg of the compound of Formula (I) three times a week to a patient in need thereof. In some embodiments, the method may include administering about 110 mg of the compound of Formula (I) three times a week to a patient in need thereof. In some embodiments, the method may include administering about 115 mg of the compound of Formula (I) three times a week to a patient in need thereof. In some embodiments, the method may include administering about 120 mg of the compound of Formula (I) three times a week to a patient in need thereof. In some embodiments, the method may include administering about 125 mg of the compound of Formula (I) three times a week to a patient in need thereof. In some embodiments, the method may include administering about 130 mg of the compound of Formula (I) three times a week to a patient in need thereof. In some embodiments, the method may include administering about 135 mg of the compound of Formula (I) three times a week to a patient in need thereof. In some embodiments, the method may include administering about 140 mg of the compound of Formula (I) three times a week to a patient in need thereof. In some embodiments, the method may include administering about 145 mg of the compound of Formula (I) three times a week to a patient in need thereof. In some embodiments, the method may include administering about 150 mg of the compound of Formula (I) three times a week to a patient in need thereof.

In some embodiments, such a disclosed method may include, administering a loading dose of the compound of Formula (I) daily to a patient in need thereof, once a week to a patient in need thereof, twice a week to a patient in need thereof, or three times a week to a patient in need thereof, for a first time period; and administering a maintenance dose of the compound of Formula (I) to a patient in need thereof daily to a patient in need thereof, once a week to a patient in need thereof, twice a week to a patient in need thereof, or three times a week to a patient in need thereof for a second time period.

In some embodiments, the loading dose is about 2 mg to about 100 mg of the compound of Formula (I) once a day. In some embodiments, the loading dose is about 5 mg to about 90 mg of the compound of Formula (I) once a day. In some embodiments, the loading dose is about 10 mg to about 80 mg of the compound of Formula (I) once a day. In some embodiments, the loading dose is about 10 mg to about 70 mg of the compound of Formula (I) once a day. In some embodiments, the loading dose is about 10 mg to about 60 mg of the compound of Formula (I) once a day. In some embodiments, the loading dose is about 10 mg to about 50 mg of the compound of Formula (I) once a day. In some embodiments, the loading dose is about 10 mg to about 40 mg of the compound of Formula (I) once a day. In some embodiments, the loading dose is about 10 mg to about 30 mg of the compound of Formula (I) once a day. In some embodiments, the loading dose is about 10 mg to about 20 mg of the compound of Formula (I) once a day. In some embodiments, the loading dose is about 2 mg of the compound of Formula (I) once a day. In some embodiments, the loading dose is about 3 mg of the compound of Formula (I) once a day. In some embodiments, the loading dose is about 4 mg of the compound of Formula (I) once a day. In some embodiments, the loading dose is about 5 mg of the compound of Formula (I) once a day. In some embodiments, the loading dose is about 10 mg of the compound of Formula (I) once a day. In some embodiments, the loading dose is about 11 mg of the compound of Formula (I) once a day. In some embodiments, the loading dose is about 12 mg of the compound of Formula (I) once a day. In some embodiments, the loading dose is about 13 mg of the compound of Formula (I) once a day. In some embodiments, the loading dose is about 14 mg of the compound of Formula (I) once a day. In some embodiments, the loading dose is about 15 mg of the compound of Formula (I) once a day. In some embodiments, the loading dose is about 16 mg of the compound of Formula (I) once a day. In some embodiments, the loading dose is about 17 mg of the compound of Formula (I) once a day. In some embodiments, the loading dose is about 18 mg of the compound of Formula (I) once a day. In some embodiments, the loading dose is about 19 mg of the compound of Formula (I) once a day. In some embodiments, the loading dose is about 20 mg of the compound of Formula (I) once a day. In some embodiments, the loading dose is about 22 mg of the compound of Formula (I) once a day. In some embodiments, the loading dose is about 24 mg of the compound of Formula (I) once a day. In some embodiments, the loading dose is about 26 mg of the compound of Formula (I) once a day. In some embodiments, the loading dose is about 28 mg of the compound of Formula (I) once a day. In some embodiments, the loading dose is about 30 mg of the compound of Formula (I) once a day. In some embodiments, the loading dose is about 32 mg of the compound of Formula (I) once a day. In some embodiments, the loading dose is about 34 mg of the compound of Formula (I) once a day. In some embodiments, the loading dose is about 36 mg of the compound of Formula (I) once a day. In some embodiments, the loading dose is about 38 mg of the compound of Formula (I) once a day. In some embodiments, the loading dose is about 40 mg of the compound of Formula (I) once a day. In some embodiments, the loading dose is about 42 mg of the compound of Formula (I) once a day. In some embodiments, the loading dose is about 44 mg of the compound of Formula (I) once a day. In some embodiments, the loading dose is about 46 mg of the compound of Formula (I) once a day. In some embodiments, the loading dose is about 48 mg of the compound of Formula (I) once a day. In some embodiments, the loading dose is about 50 mg of the compound of Formula (I) once a day.

In some embodiments, the loading dose is about 2 mg to about 100 mg of the compound of Formula (I) once a day for one day. In some embodiments, the loading dose is about 5 mg to about 90 mg of the compound of Formula (I) once a day for one day. In some embodiments, the loading dose is about 10 mg to about 80 mg of the compound of Formula (I) once a day for one day. In some embodiments, the loading dose is about 10 mg to about 70 mg of the compound of Formula (I) once a day for one day. In some embodiments, the loading dose is about 10 mg to about 60 mg of the compound of Formula (I) once a day for one day. In some embodiments, the loading dose is about 10 mg to about 50 mg of the compound of Formula (I) once a day for one day. In some embodiments, the loading dose is about 10 mg to about 40 mg of the compound of Formula (I) once a day for one day. In some embodiments, the loading dose is about 10 mg to about 30 mg of the compound of Formula (I) once a day for one day. In some embodiments, the loading dose is about 10 mg to about 20 mg of the compound of Formula (I) once a day for one day. In some embodiments, the loading dose is about 2 mg of the compound of Formula (I) once a day for one day. In some embodiments, the loading dose is about 3 mg of the compound of Formula (I) once a day for one day. In some embodiments, the loading dose is about 4 mg of the compound of Formula (I) once a day for one day. In some embodiments, the loading dose is about 5 mg of the compound of Formula (I) once a day for one day. In some embodiments, the loading dose is about 10 mg of the compound of Formula (I) once a day for one day. In some embodiments, the loading dose is about 11 mg of the compound of Formula (I) once a day for one day. In some embodiments, the loading dose is about 12 mg of the compound of Formula (I) once a day for one day. In some embodiments, the loading dose is about 13 mg of the compound of Formula (I) once a day for one day. In some embodiments, the loading dose is about 14 mg of the compound of Formula (I) once a day for one day. In some embodiments, the loading dose is about 15 mg of the compound of Formula (I) once a day for one day. In some embodiments, the loading dose is about 16 mg of the compound of Formula (I) once a day for one day. In some embodiments, the loading dose is about 17 mg of the compound of Formula (I) once a day for one day. In some embodiments, the loading dose is about 18 mg of the compound of Formula (I) once a day for one day. In some embodiments, the loading dose is about 19 mg of the compound of Formula (I) once a day for one day. In some embodiments, the loading dose is about 20 mg of the compound of Formula (I) once a day for one day. In some embodiments, the loading dose is about 22 mg of the compound of Formula (I) once a day for one day. In some embodiments, the loading dose is about 24 mg of the compound of Formula (I) once a day for one day. In some embodiments, the loading dose is about 26 mg of the compound of Formula (I) once a day for one day. In some embodiments, the loading dose is about 28 mg of the compound of Formula (I) once a day for one day. In some embodiments, the loading dose is about 30 mg of the compound of Formula (I) once a day for one day. In some embodiments, the loading dose is about 32 mg of the compound of Formula (I) once a day for one day. In some embodiments, the loading dose is about 34 mg of the compound of Formula (I) once a day for one day. In some embodiments, the loading dose is about 36 mg of the compound of Formula (I) once a day for one day. In some embodiments, the loading dose is about 38 mg of the compound of Formula (I) once a day for one day. In some embodiments, the loading dose is about 40 mg of the compound of Formula (I) once a day for one day. In some embodiments, the loading dose is about 42 mg of the compound of Formula (I) once a day for one day. In some embodiments, the loading dose is about 44 mg of the compound of Formula (I) once a day for one day. In some embodiments, the loading dose is about 46 mg of the compound of Formula (I) once a day for one day. In some embodiments, the loading dose is about 48 mg of the compound of Formula (I) once a day for one day. In some embodiments, the loading dose is about 50 mg of the compound of Formula (I) once a day for one day.

In some embodiments, the loading dose is about 2 mg to about 100 mg of the compound of Formula (I) once a day for two days. In some embodiments, the loading dose is about 5 mg to about 90 mg of the compound of Formula (I) once a day for two days. In some embodiments, the loading dose is about 10 mg to about 80 mg of the compound of Formula (I) once a day for two days. In some embodiments, the loading dose is about 10 mg to about 70 mg of the compound of Formula (I) once a day for two days. In some embodiments, the loading dose is about 10 mg to about 60 mg of the compound of Formula (I) once a day for two days. In some embodiments, the loading dose is about 10 mg to about 50 mg of the compound of Formula (I) once a day for two days. In some embodiments, the loading dose is about 10 mg to about 40 mg of the compound of Formula (I) once a day for two days. In some embodiments, the loading dose is about 10 mg to about 30 mg of the compound of Formula (I) once a day for two days. In some embodiments, the loading dose is about 10 mg to about 20 mg of the compound of Formula (I) once a day for two days. In some embodiments, the loading dose is about 2 mg of the compound of Formula (I) once a day for two days. In some embodiments, the loading dose is about 3 mg of the compound of Formula (I) once a day for two days. In some embodiments, the loading dose is about 4 mg of the compound of Formula (I) once a day for two days. In some embodiments, the loading dose is about 5 mg of the compound of Formula (I) once a day for two days. In some embodiments, the loading dose is about 10 mg of the compound of Formula (I) once a day for two days. In some embodiments, the loading dose is about 11 mg of the compound of Formula (I) once a day for two days. In some embodiments, the loading dose is about 12 mg of the compound of Formula (I) once a day for two days. In some embodiments, the loading dose is about 13 mg of the compound of Formula (I) once a day for two days. In some embodiments, the loading dose is about 14 mg of the compound of Formula (I) once a day for two days. In some embodiments, the loading dose is about 15 mg of the compound of Formula (I) once a day for two days. In some embodiments, the loading dose is about 16 mg of the compound of Formula (I) once a day for two days. In some embodiments, the loading dose is about 17 mg of the compound of Formula (I) once a day for two days. In some embodiments, the loading dose is about 18 mg of the compound of Formula (I) once a day for two days. In some embodiments, the loading dose is about 19 mg of the compound of Formula (I) once a day for two days. In some embodiments, the loading dose is about 20 mg of the compound of Formula (I) once a day for two days. In some embodiments, the loading dose is about 22 mg of the compound of Formula (I) once a day for two days. In some embodiments, the loading dose is about 24 mg of the compound of Formula (I) once a day for two days. In some embodiments, the loading dose is about 26 mg of the compound of Formula (I) once a day for two days. In some embodiments, the loading dose is about 28 mg of the compound of Formula (I) once a day for two days. In some embodiments, the loading dose is about 30 mg of the compound of Formula (I) once a day for two days. In some embodiments, the loading dose is about 32 mg of the compound of Formula (I) once a day for two days. In some embodiments, the loading dose is about 34 mg of the compound of Formula (I) once a day for two days. In some embodiments, the loading dose is about 36 mg of the compound of Formula (I) once a day for two days. In some embodiments, the loading dose is about 38 mg of the compound of Formula (I) once a day for two days. In some embodiments, the loading dose is about 40 mg of the compound of Formula (I) once a day for two days. In some embodiments, the loading dose is about 42 mg of the compound of Formula (I) once a day for two days. In some embodiments, the loading dose is about 44 mg of the compound of Formula (I) once a day for two days. In some embodiments, the loading dose is about 46 mg of the compound of Formula (I) once a day for two days. In some embodiments, the loading dose is about 48 mg of the compound of Formula (I) once a day for two days. In some embodiments, the loading dose is about 50 mg of the compound of Formula (I) once a day for two days.

In some embodiments, the loading dose is about 2 mg to about 100 mg of the compound of Formula (I) once a day for three days. In some embodiments, the loading dose is about 5 mg to about 90 mg of the compound of Formula (I) once a day for three days. In some embodiments, the loading dose is about 10 mg to about 80 mg of the compound of Formula (I) once a day for three days. In some embodiments, the loading dose is about 10 mg to about 70 mg of the compound of Formula (I) once a day for three days. In some embodiments, the loading dose is about 10 mg to about 60 mg of the compound of Formula (I) once a day for three days. In some embodiments, the loading dose is about 10 mg to about 50 mg of the compound of Formula (I) once a day for three days. In some embodiments, the loading dose is about 10 mg to about 40 mg of the compound of Formula (I) once a day for three days. In some embodiments, the loading dose is about 10 mg to about 30 mg of the compound of Formula (I) once a day for three days. In some embodiments, the loading dose is about 10 mg to about 20 mg of the compound of Formula (I) once a day for three days. In some embodiments, the loading dose is about 2 mg of the compound of Formula (I) once a day for three days. In some embodiments, the loading dose is about 3 mg of the compound of Formula (I) once a day for three days. In some embodiments, the loading dose is about 4 mg of the compound of Formula (I) once a day for three days. In some embodiments, the loading dose is about 5 mg of the compound of Formula (I) once a day for three days. In some embodiments, the loading dose is about 10 mg of the compound of Formula (I) once a day for three days. In some embodiments, the loading dose is about 11 mg of the compound of Formula (I) once a day for three days. In some embodiments, the loading dose is about 12 mg of the compound of Formula (I) once a day for three days. In some embodiments, the loading dose is about 13 mg of the compound of Formula (I) once a day for three days. In some embodiments, the loading dose is about 14 mg of the compound of Formula (I) once a day for three days. In some embodiments, the loading dose is about 15 mg of the compound of Formula (I) once a day for three days. In some embodiments, the loading dose is about 16 mg of the compound of Formula (I) once a day for three days. In some embodiments, the loading dose is about 17 mg of the compound of Formula (I) once a day for three days. In some embodiments, the loading dose is about 18 mg of the compound of Formula (I) once a day for three days. In some embodiments, the loading dose is about 19 mg of the compound of Formula (I) once a day for three days. In some embodiments, the loading dose is about 20 mg of the compound of Formula (I) once a day for three days. In some embodiments, the loading dose is about 22 mg of the compound of Formula (I) once a day for three days. In some embodiments, the loading dose is about 24 mg of the compound of Formula (I) once a day for three days. In some embodiments, the loading dose is about 26 mg of the compound of Formula (I) once a day for three days. In some embodiments, the loading dose is about 28 mg of the compound of Formula (I) once a day for three days. In some embodiments, the loading dose is about 30 mg of the compound of Formula (I) once a day for three days. In some embodiments, the loading dose is about 32 mg of the compound of Formula (I) once a day for three days. In some embodiments, the loading dose is about 34 mg of the compound of Formula (I) once a day for three days. In some embodiments, the loading dose is about 36 mg of the compound of Formula (I) once a day for three days. In some embodiments, the loading dose is about 38 mg of the compound of Formula (I) once a day for three days. In some embodiments, the loading dose is about 40 mg of the compound of Formula (I) once a day for three days. In some embodiments, the loading dose is about 42 mg of the compound of Formula (I) once a day for three days. In some embodiments, the loading dose is about 44 mg of the compound of Formula (I) once a day for three days. In some embodiments, the loading dose is about 46 mg of the compound of Formula (I) once a day for three days. In some embodiments, the loading dose is about 48 mg of the compound of Formula (I) once a day for three days. In some embodiments, the loading dose is about 50 mg of the compound of Formula (I) once a day for three days.

In some embodiments, the loading dose is about 2 mg to about 100 mg of the compound of Formula (I) once a day for four days. In some embodiments, the loading dose is about 5 mg to about 90 mg of the compound of Formula (I) once a day for four days. In some embodiments, the loading dose is about 10 mg to about 80 mg of the compound of Formula (I) once a day for four days. In some embodiments, the loading dose is about 10 mg to about 70 mg of the compound of Formula (I) once a day for four days. In some embodiments, the loading dose is about 10 mg to about 60 mg of the compound of Formula (I) once a day for four days. In some embodiments, the loading dose is about 10 mg to about 50 mg of the compound of Formula (I) once a day for four days. In some embodiments, the loading dose is about 10 mg to about 40 mg of the compound of Formula (I) once a day for four days. In some embodiments, the loading dose is about 10 mg to about 30 mg of the compound of Formula (I) once a day for four days. In some embodiments, the loading dose is about 10 mg to about 20 mg of the compound of Formula (I) once a day for four days. In some embodiments, the loading dose is about 2 mg of the compound of Formula (I) once a day for four days. In some embodiments, the loading dose is about 3 mg of the compound of Formula (I) once a day for four days. In some embodiments, the loading dose is about 4 mg of the compound of Formula (I) once a day for four days. In some embodiments, the loading dose is about 5 mg of the compound of Formula (I) once a day for four days. In some embodiments, the loading dose is about 10 mg of the compound of Formula (I) once a day for four days. In some embodiments, the loading dose is about 11 mg of the compound of Formula (I) once a day for four days. In some embodiments, the loading dose is about 12 mg of the compound of Formula (I) once a day for four days. In some embodiments, the loading dose is about 13 mg of the compound of Formula (I) once a day for four days. In some embodiments, the loading dose is about 14 mg of the compound of Formula (I) once a day for four days. In some embodiments, the loading dose is about 15 mg of the compound of Formula (I) once a day for four days. In some embodiments, the loading dose is about 16 mg of the compound of Formula (I) once a day for four days. In some embodiments, the loading dose is about 17 mg of the compound of Formula (I) once a day for four days. In some embodiments, the loading dose is about 18 mg of the compound of Formula (I) once a day for four days. In some embodiments, the loading dose is about 19 mg of the compound of Formula (I) once a day for four days. In some embodiments, the loading dose is about 20 mg of the compound of Formula (I) once a day for four days. In some embodiments, the loading dose is about 22 mg of the compound of Formula (I) once a day for four days. In some embodiments, the loading dose is about 24 mg of the compound of Formula (I) once a day for four days. In some embodiments, the loading dose is about 26 mg of the compound of Formula (I) once a day for four days. In some embodiments, the loading dose is about 28 mg of the compound of Formula (I) once a day for four days. In some embodiments, the loading dose is about 30 mg of the compound of Formula (I) once a day for four days. In some embodiments, the loading dose is about 32 mg of the compound of Formula (I) once a day for four days. In some embodiments, the loading dose is about 34 mg of the compound of Formula (I) once a day for four days. In some embodiments, the loading dose is about 36 mg of the compound of Formula (I) once a day for four days. In some embodiments, the loading dose is about 38 mg of the compound of Formula (I) once a day for four days. In some embodiments, the loading dose is about 40 mg of the compound of Formula (I) once a day for four days. In some embodiments, the loading dose is about 42 mg of the compound of Formula (I) once a day for four days. In some embodiments, the loading dose is about 44 mg of the compound of Formula (I) once a day for four days. In some embodiments, the loading dose is about 46 mg of the compound of Formula (I) once a day for four days. In some embodiments, the loading dose is about 48 mg of the compound of Formula (I) once a day for four days. In some embodiments, the loading dose is about 50 mg of the compound of Formula (I) once a day for four days.

In some embodiments, the loading dose is about 2 mg to about 100 mg of the compound of Formula (I) once a day for five days. In some embodiments, the loading dose is about 5 mg to about 90 mg of the compound of Formula (I) once a day for five days. In some embodiments, the loading dose is about 10 mg to about 80 mg of the compound of Formula (I) once a day for five days. In some embodiments, the loading dose is about 10 mg to about 70 mg of the compound of Formula (I) once a day for five days. In some embodiments, the loading dose is about 10 mg to about 60 mg of the compound of Formula (I) once a day for five days. In some embodiments, the loading dose is about 10 mg to about 50 mg of the compound of Formula (I) once a day for five days. In some embodiments, the loading dose is about 10 mg to about 40 mg of the compound of Formula (I) once a day for five days. In some embodiments, the loading dose is about 10 mg to about 30 mg of the compound of Formula (I) once a day for five days. In some embodiments, the loading dose is about 10 mg to about 20 mg of the compound of Formula (I) once a day for five days. In some embodiments, the loading dose is about 2 mg of the compound of Formula (I) once a day for five days. In some embodiments, the loading dose is about 3 mg of the compound of Formula (I) once a day for five days. In some embodiments, the loading dose is about 4 mg of the compound of Formula (I) once a day for five days. In some embodiments, the loading dose is about 5 mg of the compound of Formula (I) once a day for five days. In some embodiments, the loading dose is about 10 mg of the compound of Formula (I) once a day for five days. In some embodiments, the loading dose is about 11 mg of the compound of Formula (I) once a day for five days. In some embodiments, the loading dose is about 12 mg of the compound of Formula (I) once a day for five days. In some embodiments, the loading dose is about 13 mg of the compound of Formula (I) once a day for five days. In some embodiments, the loading dose is about 14 mg of the compound of Formula (I) once a day for five days. In some embodiments, the loading dose is about 15 mg of the compound of Formula (I) once a day for five days. In some embodiments, the loading dose is about 16 mg of the compound of Formula (I) once a day for five days. In some embodiments, the loading dose is about 17 mg of the compound of Formula (I) once a day for five days. In some embodiments, the loading dose is about 18 mg of the compound of Formula (I) once a day for five days. In some embodiments, the loading dose is about 19 mg of the compound of Formula (I) once a day for five days. In some embodiments, the loading dose is about 20 mg of the compound of Formula (I) once a day for five days. In some embodiments, the loading dose is about 22 mg of the compound of Formula (I) once a day for five days. In some embodiments, the loading dose is about 24 mg of the compound of Formula (I) once a day for five days. In some embodiments, the loading dose is about 26 mg of the compound of Formula (I) once a day for five days. In some embodiments, the loading dose is about 28 mg of the compound of Formula (I) once a day for five days. In some embodiments, the loading dose is about 30 mg of the compound of Formula (I) once a day for five days. In some embodiments, the loading dose is about 32 mg of the compound of Formula (I) once a day for five days. In some embodiments, the loading dose is about 34 mg of the compound of Formula (I) once a day for five days. In some embodiments, the loading dose is about 36 mg of the compound of Formula (I) once a day for five days. In some embodiments, the loading dose is about 38 mg of the compound of Formula (I) once a day for five days. In some embodiments, the loading dose is about 40 mg of the compound of Formula (I) once a day for five days. In some embodiments, the loading dose is about 42 mg of the compound of Formula (I) once a day for five days. In some embodiments, the loading dose is about 44 mg of the compound of Formula (I) once a day for five days. In some embodiments, the loading dose is about 46 mg of the compound of Formula (I) once a day for five days. In some embodiments, the loading dose is about 48 mg of the compound of Formula (I) once a day for five days. In some embodiments, the loading dose is about 50 mg of the compound of Formula (I) once a day for five days.

In some embodiments, the loading dose is about 2 mg to about 100 mg of the compound of Formula (I) once a day for six days. In some embodiments, the loading dose is about 5 mg to about 90 mg of the compound of Formula (I) once a day for six days. In some embodiments, the loading dose is about 10 mg to about 80 mg of the compound of Formula (I) once a day for six days. In some embodiments, the loading dose is about 10 mg to about 70 mg of the compound of Formula (I) once a day for six days. In some embodiments, the loading dose is about 10 mg to about 60 mg of the compound of Formula (I) once a day for six days. In some embodiments, the loading dose is about 10 mg to about 50 mg of the compound of Formula (I) once a day for six days. In some embodiments, the loading dose is about 10 mg to about 40 mg of the compound of Formula (I) once a day for six days. In some embodiments, the loading dose is about 10 mg to about 30 mg of the compound of Formula (I) once a day for six days. In some embodiments, the loading dose is about 10 mg to about 20 mg of the compound of Formula (I) once a day for six days. In some embodiments, the loading dose is about 2 mg of the compound of Formula (I) once a day for six days. In some embodiments, the loading dose is about 3 mg of the compound of Formula (I) once a day for six days. In some embodiments, the loading dose is about 4 mg of the compound of Formula (I) once a day for six days. In some embodiments, the loading dose is about 5 mg of the compound of Formula (I) once a day for six days. In some embodiments, the loading dose is about 10 mg of the compound of Formula (I) once a day for six days. In some embodiments, the loading dose is about 11 mg of the compound of Formula (I) once a day for six days. In some embodiments, the loading dose is about 12 mg of the compound of Formula (I) once a day for six days. In some embodiments, the loading dose is about 13 mg of the compound of Formula (I) once a day for six days. In some embodiments, the loading dose is about 14 mg of the compound of Formula (I) once a day for six days. In some embodiments, the loading dose is about 15 mg of the compound of Formula (I) once a day for six days. In some embodiments, the loading dose is about 16 mg of the compound of Formula (I) once a day for six days. In some embodiments, the loading dose is about 17 mg of the compound of Formula (I) once a day for six days. In some embodiments, the loading dose is about 18 mg of the compound of Formula (I) once a day for six days. In some embodiments, the loading dose is about 19 mg of the compound of Formula (I) once a day for six days. In some embodiments, the loading dose is about 20 mg of the compound of Formula (I) once a day for six days. In some embodiments, the loading dose is about 22 mg of the compound of Formula (I) once a day for six days. In some embodiments, the loading dose is about 24 mg of the compound of Formula (I) once a day for six days. In some embodiments, the loading dose is about 26 mg of the compound of Formula (I) once a day for six days. In some embodiments, the loading dose is about 28 mg of the compound of Formula (I) once a day for six days. In some embodiments, the loading dose is about 30 mg of the compound of Formula (I) once a day for six days. In some embodiments, the loading dose is about 32 mg of the compound of Formula (I) once a day for six days. In some embodiments, the loading dose is about 34 mg of the compound of Formula (I) once a day for six days. In some embodiments, the loading dose is about 36 mg of the compound of Formula (I) once a day for six days. In some embodiments, the loading dose is about 38 mg of the compound of Formula (I) once a day for six days. In some embodiments, the loading dose is about 40 mg of the compound of Formula (I) once a day for six days. In some embodiments, the loading dose is about 42 mg of the compound of Formula (I) once a day for six days. In some embodiments, the loading dose is about 44 mg of the compound of Formula (I) once a day for six days. In some embodiments, the loading dose is about 46 mg of the compound of Formula (I) once a day for six days. In some embodiments, the loading dose is about 48 mg of the compound of Formula (I) once a day for six days. In some embodiments, the loading dose is about 50 mg of the compound of Formula (I) once a day for six days.

In some embodiments, the loading dose is about 2 mg to about 100 mg of the compound of Formula (I) once a day for seven days. In some embodiments, the loading dose is about 5 mg to about 90 mg of the compound of Formula (I) once a day for seven days. In some embodiments, the loading dose is about 10 mg to about 80 mg of the compound of Formula (I) once a day for seven days. In some embodiments, the loading dose is about 10 mg to about 70 mg of the compound of Formula (I) once a day for seven days. In some embodiments, the loading dose is about 10 mg to about 60 mg of the compound of Formula (I) once a day for seven days. In some embodiments, the loading dose is about 10 mg to about 50 mg of the compound of Formula (I) once a day for seven days. In some embodiments, the loading dose is about 10 mg to about 40 mg of the compound of Formula (I) once a day for seven days. In some embodiments, the loading dose is about 10 mg to about 30 mg of the compound of Formula (I) once a day for seven days. In some embodiments, the loading dose is about 10 mg to about 20 mg of the compound of Formula (I) once a day for seven days. In some embodiments, the loading dose is about 2 mg of the compound of Formula (I) once a day for seven days. In some embodiments, the loading dose is about 3 mg of the compound of Formula (I) once a day for seven days. In some embodiments, the loading dose is about 4 mg of the compound of Formula (I) once a day for seven days. In some embodiments, the loading dose is about 5 mg of the compound of Formula (I) once a day for seven days. In some embodiments, the loading dose is about 10 mg of the compound of Formula (I) once a day for seven days. In some embodiments, the loading dose is about 11 mg of the compound of Formula (I) once a day for seven days. In some embodiments, the loading dose is about 12 mg of the compound of Formula (I) once a day for seven days. In some embodiments, the loading dose is about 13 mg of the compound of Formula (I) once a day for seven days. In some embodiments, the loading dose is about 14 mg of the compound of Formula (I) once a day for seven days. In some embodiments, the loading dose is about 15 mg of the compound of Formula (I) once a day for seven days. In some embodiments, the loading dose is about 16 mg of the compound of Formula (I) once a day for seven days. In some embodiments, the loading dose is about 17 mg of the compound of Formula (I) once a day for seven days. In some embodiments, the loading dose is about 18 mg of the compound of Formula (I) once a day for seven days. In some embodiments, the loading dose is about 19 mg of the compound of Formula (I) once a day for seven days. In some embodiments, the loading dose is about 20 mg of the compound of Formula (I) once a day for seven days. In some embodiments, the loading dose is about 22 mg of the compound of Formula (I) once a day for seven days. In some embodiments, the loading dose is about 24 mg of the compound of Formula (I) once a day for seven days. In some embodiments, the loading dose is about 26 mg of the compound of Formula (I) once a day for seven days. In some embodiments, the loading dose is about 28 mg of the compound of Formula (I) once a day for seven days. In some embodiments, the loading dose is about 30 mg of the compound of Formula (I) once a day for seven days. In some embodiments, the loading dose is about 32 mg of the compound of Formula (I) once a day for seven days. In some embodiments, the loading dose is about 34 mg of the compound of Formula (I) once a day for seven days. In some embodiments, the loading dose is about 36 mg of the compound of Formula (I) once a day for seven days. In some embodiments, the loading dose is about 38 mg of the compound of Formula (I) once a day for seven days. In some embodiments, the loading dose is about 40 mg of the compound of Formula (I) once a day for seven days. In some embodiments, the loading dose is about 42 mg of the compound of Formula (I) once a day for seven days. In some embodiments, the loading dose is about 44 mg of the compound of Formula (I) once a day for seven days. In some embodiments, the loading dose is about 46 mg of the compound of Formula (I) once a day for seven days. In some embodiments, the loading dose is about 48 mg of the compound of Formula (I) once a day for seven days. In some embodiments, the loading dose is about 50 mg of the compound of Formula (I) once a day for seven days.

In some embodiments, the loading dose is about 2 mg to about 100 mg of the compound of Formula (I) once a day for eight days. In some embodiments, the loading dose is about 5 mg to about 90 mg of the compound of Formula (I) once a day for eight days. In some embodiments, the loading dose is about 10 mg to about 80 mg of the compound of Formula (I) once a day for eight days. In some embodiments, the loading dose is about 10 mg to about 70 mg of the compound of Formula (I) once a day for eight days. In some embodiments, the loading dose is about 10 mg to about 60 mg of the compound of Formula (I) once a day for eight days. In some embodiments, the loading dose is about 10 mg to about 50 mg of the compound of Formula (I) once a day for eight days. In some embodiments, the loading dose is about 10 mg to about 40 mg of the compound of Formula (I) once a day for eight days. In some embodiments, the loading dose is about 10 mg to about 30 mg of the compound of Formula (I) once a day for eight days. In some embodiments, the loading dose is about 10 mg to about 20 mg of the compound of Formula (I) once a day for eight days. In some embodiments, the loading dose is about 2 mg of the compound of Formula (I) once a day for eight days. In some embodiments, the loading dose is about 3 mg of the compound of Formula (I) once a day for eight days. In some embodiments, the loading dose is about 4 mg of the compound of Formula (I) once a day for eight days. In some embodiments, the loading dose is about 5 mg of the compound of Formula (I) once a day for eight days. In some embodiments, the loading dose is about 10 mg of the compound of Formula (I) once a day for eight days. In some embodiments, the loading dose is about 11 mg of the compound of Formula (I) once a day for eight days. In some embodiments, the loading dose is about 12 mg of the compound of Formula (I) once a day for eight days. In some embodiments, the loading dose is about 13 mg of the compound of Formula (I) once a day for eight days. In some embodiments, the loading dose is about 14 mg of the compound of Formula (I) once a day for eight days. In some embodiments, the loading dose is about 15 mg of the compound of Formula (I) once a day for eight days. In some embodiments, the loading dose is about 16 mg of the compound of Formula (I) once a day for eight days. In some embodiments, the loading dose is about 17 mg of the compound of Formula (I) once a day for eight days. In some embodiments, the loading dose is about 18 mg of the compound of Formula (I) once a day for eight days. In some embodiments, the loading dose is about 19 mg of the compound of Formula (I) once a day for eight days. In some embodiments, the loading dose is about 20 mg of the compound of Formula (I) once a day for eight days. In some embodiments, the loading dose is about 22 mg of the compound of Formula (I) once a day for eight days. In some embodiments, the loading dose is about 24 mg of the compound of Formula (I) once a day for eight days. In some embodiments, the loading dose is about 26 mg of the compound of Formula (I) once a day for eight days. In some embodiments, the loading dose is about 28 mg of the compound of Formula (I) once a day for eight days. In some embodiments, the loading dose is about 30 mg of the compound of Formula (I) once a day for eight days. In some embodiments, the loading dose is about 32 mg of the compound of Formula (I) once a day for eight days. In some embodiments, the loading dose is about 34 mg of the compound of Formula (I) once a day for eight days. In some embodiments, the loading dose is about 36 mg of the compound of Formula (I) once a day for eight days. In some embodiments, the loading dose is about 38 mg of the compound of Formula (I) once a day for eight days. In some embodiments, the loading dose is about 40 mg of the compound of Formula (I) once a day for eight days. In some embodiments, the loading dose is about 42 mg of the compound of Formula (I) once a day for eight days. In some embodiments, the loading dose is about 44 mg of the compound of Formula (I) once a day for eight days. In some embodiments, the loading dose is about 46 mg of the compound of Formula (I) once a day for eight days. In some embodiments, the loading dose is about 48 mg of the compound of Formula (I) once a day for eight days. In some embodiments, the loading dose is about 50 mg of the compound of Formula (I) once a day for eight days.

In some embodiments, the loading dose is about 2 mg to about 100 mg of the compound of Formula (I) once a day for nine days. In some embodiments, the loading dose is about 5 mg to about 90 mg of the compound of Formula (I) once a day for nine days. In some embodiments, the loading dose is about 10 mg to about 80 mg of the compound of Formula (I) once a day for nine days. In some embodiments, the loading dose is about 10 mg to about 70 mg of the compound of Formula (I) once a day for nine days. In some embodiments, the loading dose is about 10 mg to about 60 mg of the compound of Formula (I) once a day for nine days. In some embodiments, the loading dose is about 10 mg to about 50 mg of the compound of Formula (I) once a day for nine days. In some embodiments, the loading dose is about 10 mg to about 40 mg of the compound of Formula (I) once a day for nine days. In some embodiments, the loading dose is about 10 mg to about 30 mg of the compound of Formula (I) once a day for nine days. In some embodiments, the loading dose is about 10 mg to about 20 mg of the compound of Formula (I) once a day for nine days. In some embodiments, the loading dose is about 2 mg of the compound of Formula (I) once a day for nine days. In some embodiments, the loading dose is about 3 mg of the compound of Formula (I) once a day for nine days. In some embodiments, the loading dose is about 4 mg of the compound of Formula (I) once a day for nine days. In some embodiments, the loading dose is about 5 mg of the compound of Formula (I) once a day for nine days. In some embodiments, the loading dose is about 10 mg of the compound of Formula (I) once a day for nine days. In some embodiments, the loading dose is about 11 mg of the compound of Formula (I) once a day for nine days. In some embodiments, the loading dose is about 12 mg of the compound of Formula (I) once a day for nine days. In some embodiments, the loading dose is about 13 mg of the compound of Formula (I) once a day for nine days. In some embodiments, the loading dose is about 14 mg of the compound of Formula (I) once a day for nine days. In some embodiments, the loading dose is about 15 mg of the compound of Formula (I) once a day for nine days. In some embodiments, the loading dose is about 16 mg of the compound of Formula (I) once a day for nine days. In some embodiments, the loading dose is about 17 mg of the compound of Formula (I) once a day for nine days. In some embodiments, the loading dose is about 18 mg of the compound of Formula (I) once a day for nine days. In some embodiments, the loading dose is about 19 mg of the compound of Formula (I) once a day for nine days. In some embodiments, the loading dose is about 20 mg of the compound of Formula (I) once a day for nine days. In some embodiments, the loading dose is about 22 mg of the compound of Formula (I) once a day for nine days. In some embodiments, the loading dose is about 24 mg of the compound of Formula (I) once a day for nine days. In some embodiments, the loading dose is about 26 mg of the compound of Formula (I) once a day for nine days. In some embodiments, the loading dose is about 28 mg of the compound of Formula (I) once a day for nine days. In some embodiments, the loading dose is about 30 mg of the compound of Formula (I) once a day for nine days. In some embodiments, the loading dose is about 32 mg of the compound of Formula (I) once a day for nine days. In some embodiments, the loading dose is about 34 mg of the compound of Formula (I) once a day for nine days. In some embodiments, the loading dose is about 36 mg of the compound of Formula (I) once a day for nine days. In some embodiments, the loading dose is about 38 mg of the compound of Formula (I) once a day for nine days. In some embodiments, the loading dose is about 40 mg of the compound of Formula (I) once a day for nine days. In some embodiments, the loading dose is about 42 mg of the compound of Formula (I) once a day for nine days. In some embodiments, the loading dose is about 44 mg of the compound of Formula (I) once a day for nine days. In some embodiments, the loading dose is about 46 mg of the compound of Formula (I) once a day for nine days. In some embodiments, the loading dose is about 48 mg of the compound of Formula (I) once a day for nine days. In some embodiments, the loading dose is about 50 mg of the compound of Formula (I) once a day for nine days.

In some embodiments, the loading dose is about 2 mg to about 100 mg of the compound of Formula (I) once a day for ten days. In some embodiments, the loading dose is about 5 mg to about 90 mg of the compound of Formula (I) once a day for ten days. In some embodiments, the loading dose is about 10 mg to about 80 mg of the compound of Formula (I) once a day for ten days. In some embodiments, the loading dose is about 10 mg to about 70 mg of the compound of Formula (I) once a day for ten days. In some embodiments, the loading dose is about 10 mg to about 60 mg of the compound of Formula (I) once a day for ten days. In some embodiments, the loading dose is about 10 mg to about 50 mg of the compound of Formula (I) once a day for ten days. In some embodiments, the loading dose is about 10 mg to about 40 mg of the compound of Formula (I) once a day for ten days. In some embodiments, the loading dose is about 10 mg to about 30 mg of the compound of Formula (I) once a day for ten days. In some embodiments, the loading dose is about 10 mg to about 20 mg of the compound of Formula (I) once a day for ten days. In some embodiments, the loading dose is about 2 mg of the compound of Formula (I) once a day for ten days. In some embodiments, the loading dose is about 3 mg of the compound of Formula (I) once a day for ten days. In some embodiments, the loading dose is about 4 mg of the compound of Formula (I) once a day for ten days. In some embodiments, the loading dose is about 5 mg of the compound of Formula (I) once a day for ten days. In some embodiments, the loading dose is about 10 mg of the compound of Formula (I) once a day for ten days. In some embodiments, the loading dose is about 11 mg of the compound of Formula (I) once a day for ten days. In some embodiments, the loading dose is about 12 mg of the compound of Formula (I) once a day for ten days. In some embodiments, the loading dose is about 13 mg of the compound of Formula (I) once a day for ten days. In some embodiments, the loading dose is about 14 mg of the compound of Formula (I) once a day for ten days. In some embodiments, the loading dose is about 15 mg of the compound of Formula (I) once a day for ten days. In some embodiments, the loading dose is about 16 mg of the compound of Formula (I) once a day for ten days. In some embodiments, the loading dose is about 17 mg of the compound of Formula (I) once a day for ten days. In some embodiments, the loading dose is about 18 mg of the compound of Formula (I) once a day for ten days. In some embodiments, the loading dose is about 19 mg of the compound of Formula (I) once a day for ten days. In some embodiments, the loading dose is about 20 mg of the compound of Formula (I) once a day for ten days. In some embodiments, the loading dose is about 22 mg of the compound of Formula (I) once a day for ten days. In some embodiments, the loading dose is about 24 mg of the compound of Formula (I) once a day for ten days. In some embodiments, the loading dose is about 26 mg of the compound of Formula (I) once a day for ten days. In some embodiments, the loading dose is about 28 mg of the compound of Formula (I) once a day for ten days. In some embodiments, the loading dose is about 30 mg of the compound of Formula (I) once a day for ten days. In some embodiments, the loading dose is about 32 mg of the compound of Formula (I) once a day for ten days. In some embodiments, the loading dose is about 34 mg of the compound of Formula (I) once a day for ten days. In some embodiments, the loading dose is about 36 mg of the compound of Formula (I) once a day for ten days. In some embodiments, the loading dose is about 38 mg of the compound of Formula (I) once a day for ten days. In some embodiments, the loading dose is about 40 mg of the compound of Formula (I) once a day for ten days. In some embodiments, the loading dose is about 42 mg of the compound of Formula (I) once a day for ten days. In some embodiments, the loading dose is about 44 mg of the compound of Formula (I) once a day for ten days. In some embodiments, the loading dose is about 46 mg of the compound of Formula (I) once a day for ten days. In some embodiments, the loading dose is about 48 mg of the compound of Formula (I) once a day for ten days. In some embodiments, the loading dose is about 50 mg of the compound of Formula (I) once a day for ten days.

In some embodiments, the loading dose is about 2 mg to about 100 mg of the compound of Formula (I) once a week. In some embodiments, the loading dose is about 5 mg to about 90 mg of the compound of Formula (I) once a week. In some embodiments, the loading dose is about 10 mg to about 80 mg of the compound of Formula (I) once a week. In some embodiments, the loading dose is about 10 mg to about 70 mg of the compound of Formula (I) once a week. In some embodiments, the loading dose is about 10 mg to about 60 mg of the compound of Formula (I) once a week. In some embodiments, the loading dose is about 10 mg to about 50 mg of the compound of Formula (I) once a week. In some embodiments, the loading dose is about 10 mg to about 40 mg of the compound of Formula (I) once a week. In some embodiments, the loading dose is about 10 mg to about 30 mg of the compound of Formula (I) once a week. In some embodiments, the loading dose is about 10 mg to about 20 mg of the compound of Formula (I) once a week. In some embodiments, the loading dose is about 2 mg of the compound of Formula (I) once a week. In some embodiments, the loading dose is about 3 mg of the compound of Formula (I) once a week. In some embodiments, the loading dose is about 4 mg of the compound of Formula (I) once a week. In some embodiments, the loading dose is about 5 mg of the compound of Formula (I) once a week. In some embodiments, the loading dose is about 10 mg of the compound of Formula (I) once a week. In some embodiments, the loading dose is about 11 mg of the compound of Formula (I) once a week. In some embodiments, the loading dose is about 12 mg of the compound of Formula (I) once a week. In some embodiments, the loading dose is about 13 mg of the compound of Formula (I) once a week. In some embodiments, the loading dose is about 14 mg of the compound of Formula (I) once a week. In some embodiments, the loading dose is about 15 mg of the compound of Formula (I) once a week. In some embodiments, the loading dose is about 16 mg of the compound of Formula (I) once a week. In some embodiments, the loading dose is about 17 mg of the compound of Formula (I) once a week. In some embodiments, the loading dose is about 18 mg of the compound of Formula (I) once a week. In some embodiments, the loading dose is about 19 mg of the compound of Formula (I) once a week. In some embodiments, the loading dose is about 20 mg of the compound of Formula (I) once a week. In some embodiments, the loading dose is about 22 mg of the compound of Formula (I) once a week. In some embodiments, the loading dose is about 24 mg of the compound of Formula (I) once a week. In some embodiments, the loading dose is about 26 mg of the compound of Formula (I) once a week. In some embodiments, the loading dose is about 28 mg of the compound of Formula (I) once a week. In some embodiments, the loading dose is about 30 mg of the compound of Formula (I) once a week. In some embodiments, the loading dose is about 32 mg of the compound of Formula (I) once a week. In some embodiments, the loading dose is about 34 mg of the compound of Formula (I) once a week. In some embodiments, the loading dose is about 36 mg of the compound of Formula (I) once a week. In some embodiments, the loading dose is about 38 mg of the compound of Formula (I) once a week. In some embodiments, the loading dose is about 40 mg of the compound of Formula (I) once a week. In some embodiments, the loading dose is about 42 mg of the compound of Formula (I) once a week. In some embodiments, the loading dose is about 44 mg of the compound of Formula (I) once a week. In some embodiments, the loading dose is about 46 mg of the compound of Formula (I) once a week. In some embodiments, the loading dose is about 48 mg of the compound of Formula (I) once a week. In some embodiments, the loading dose is about 50 mg of the compound of Formula (I) once a week.

In some embodiments, the loading dose is about 2 mg to about 100 mg of the compound of Formula (I) twice a week. In some embodiments, the loading dose is about 5 mg to about 90 mg of the compound of Formula (I) twice a week. In some embodiments, the loading dose is about 10 mg to about 80 mg of the compound of Formula (I) twice a week. In some embodiments, the loading dose is about 10 mg to about 70 mg of the compound of Formula (I) twice a week. In some embodiments, the loading dose is about 10 mg to about 60 mg of the compound of Formula (I) twice a week. In some embodiments, the loading dose is about 10 mg to about 50 mg of the compound of Formula (I) twice a week. In some embodiments, the loading dose is about 10 mg to about 40 mg of the compound of Formula (I) twice a week. In some embodiments, the loading dose is about 10 mg to about 30 mg of the compound of Formula (I) twice a week. In some embodiments, the loading dose is about 10 mg to about 20 mg of the compound of Formula (I) twice a week. In some embodiments, the loading dose is about 2 mg of the compound of Formula (I) twice a week. In some embodiments, the loading dose is about 3 mg of the compound of Formula (I) twice a week. In some embodiments, the loading dose is about 4 mg of the compound of Formula (I) twice a week. In some embodiments, the loading dose is about 5 mg of the compound of Formula (I) twice a week. In some embodiments, the loading dose is about 10 mg of the compound of Formula (I) twice a week. In some embodiments, the loading dose is about 11 mg of the compound of Formula (I) twice a week. In some embodiments, the loading dose is about 12 mg of the compound of Formula (I) twice a week. In some embodiments, the loading dose is about 13 mg of the compound of Formula (I) twice a week. In some embodiments, the loading dose is about 14 mg of the compound of Formula (I) twice a week. In some embodiments, the loading dose is about 15 mg of the compound of Formula (I) twice a week. In some embodiments, the loading dose is about 16 mg of the compound of Formula (I) twice a week. In some embodiments, the loading dose is about 17 mg of the compound of Formula (I) twice a week. In some embodiments, the loading dose is about 18 mg of the compound of Formula (I) twice a week. In some embodiments, the loading dose is about 19 mg of the compound of Formula (I) twice a week. In some embodiments, the loading dose is about 20 mg of the compound of Formula (I) twice a week. In some embodiments, the loading dose is about 22 mg of the compound of Formula (I) twice a week. In some embodiments, the loading dose is about 24 mg of the compound of Formula (I) twice a week. In some embodiments, the loading dose is about 26 mg of the compound of Formula (I) twice a week. In some embodiments, the loading dose is about 28 mg of the compound of Formula (I) twice a week. In some embodiments, the loading dose is about 30 mg of the compound of Formula (I) twice a week. In some embodiments, the loading dose is about 32 mg of the compound of Formula (I) twice a week. In some embodiments, the loading dose is about 34 mg of the compound of Formula (I) twice a week. In some embodiments, the loading dose is about 36 mg of the compound of Formula (I) twice a week. In some embodiments, the loading dose is about 38 mg of the compound of Formula (I) twice a week. In some embodiments, the loading dose is about 40 mg of the compound of Formula (I) twice a week. In some embodiments, the loading dose is about 42 mg of the compound of Formula (I) twice a week. In some embodiments, the loading dose is about 44 mg of the compound of Formula (I) twice a week. In some embodiments, the loading dose is about 46 mg of the compound of Formula (I) twice a week. In some embodiments, the loading dose is about 48 mg of the compound of Formula (I) twice a week. In some embodiments, the loading dose is about 50 mg of the compound of Formula (I) twice a week.

In some embodiments, the loading dose is about 2 mg to about 100 mg of the compound of Formula (I) three times a week. In some embodiments, the loading dose is about 5 mg to about 90 mg of the compound of Formula (I) three times a week. In some embodiments, the loading dose is about 10 mg to about 80 mg of the compound of Formula (I) three times a week. In some embodiments, the loading dose is about 10 mg to about 70 mg of the compound of Formula (I) three times a week. In some embodiments, the loading dose is about 10 mg to about 60 mg of the compound of Formula (I) three times a week. In some embodiments, the loading dose is about 10 mg to about 50 mg of the compound of Formula (I) three times a week. In some embodiments, the loading dose is about 10 mg to about 40 mg of the compound of Formula (I) three times a week. In some embodiments, the loading dose is about 10 mg to about 30 mg of the compound of Formula (I) three times a week. In some embodiments, the loading dose is about 10 mg to about 20 mg of the compound of Formula (I) three times a week. In some embodiments, the loading dose is about 2 mg of the compound of Formula (I) three times a week. In some embodiments, the loading dose is about 3 mg of the compound of Formula (I) three times a week. In some embodiments, the loading dose is about 4 mg of the compound of Formula (I) three times a week. In some embodiments, the loading dose is about 5 mg of the compound of Formula (I) three times a week. In some embodiments, the loading dose is about 10 mg of the compound of Formula (I) three times a week. In some embodiments, the loading dose is about 11 mg of the compound of Formula (I) three times a week. In some embodiments, the loading dose is about 12 mg of the compound of Formula (I) three times a week. In some embodiments, the loading dose is about 13 mg of the compound of Formula (I) three times a week. In some embodiments, the loading dose is about 14 mg of the compound of Formula (I) three times a week. In some embodiments, the loading dose is about 15 mg of the compound of Formula (I) three times a week. In some embodiments, the loading dose is about 16 mg of the compound of Formula (I) three times a week. In some embodiments, the loading dose is about 17 mg of the compound of Formula (I) three times a week. In some embodiments, the loading dose is about 18 mg of the compound of Formula (I) three times a week. In some embodiments, the loading dose is about 19 mg of the compound of Formula (I) three times a week. In some embodiments, the loading dose is about 20 mg of the compound of Formula (I) three times a week. In some embodiments, the loading dose is about 22 mg of the compound of Formula (I) three times a week. In some embodiments, the loading dose is about 24 mg of the compound of Formula (I) three times a week. In some embodiments, the loading dose is about 26 mg of the compound of Formula (I) three times a week. In some embodiments, the loading dose is about 28 mg of the compound of Formula (I) three times a week. In some embodiments, the loading dose is about 30 mg of the compound of Formula (I) three times a week. In some embodiments, the loading dose is about 32 mg of the compound of Formula (I) three times a week. In some embodiments, the loading dose is about 34 mg of the compound of Formula (I) three times a week. In some embodiments, the loading dose is about 36 mg of the compound of Formula (I) three times a week. In some embodiments, the loading dose is about 38 mg of the compound of Formula (I) three times a week. In some embodiments, the loading dose is about 40 mg of the compound of Formula (I) three times a week. In some embodiments, the loading dose is about 42 mg of the compound of Formula (I) three times a week. In some embodiments, the loading dose is about 44 mg of the compound of Formula (I) three times a week. In some embodiments, the loading dose is about 46 mg of the compound of Formula (I) three times a week. In some embodiments, the loading dose is about 48 mg of the compound of Formula (I) three times a week. In some embodiments, the loading dose is about 50 mg of the compound of Formula (I) three times a week.

In some embodiments, administering a maintenance dose may include administering about 2 mg to about 150 mg of the compound of Formula (I) daily to a patient in need thereof. In some embodiments, the maintenance dose is about 2 mg to about 100 mg of the compound of Formula (I) daily. In some embodiments, the maintenance dose is about 5 mg to about 90 mg of the compound of Formula (I) daily. In some embodiments, the maintenance dose is about 10 mg to about 80 mg of the compound of Formula (I) daily. In some embodiments, the maintenance dose is about 10 mg to about 70 mg of the compound of Formula (I) daily. In some embodiments, the maintenance dose is about 10 mg to about 60 mg of the compound of Formula (I) daily. In some embodiments, the maintenance dose is about 10 mg to about 50 mg of the compound of Formula (I) daily. In some embodiments, the maintenance dose is about 10 mg to about 40 mg of the compound of Formula (I) daily. In some embodiments, the maintenance dose is about 10 mg to about 30 mg of the compound of Formula (I) daily. In some embodiments, the maintenance dose is about 10 mg to about 20 mg of the compound of Formula (I) daily. In some embodiments, the maintenance dose is about 2 mg of the compound of Formula (I) daily. In some embodiments, the maintenance dose is about 3 mg of the compound of Formula (I) daily. In some embodiments, the maintenance dose is about 4 mg of the compound of Formula (I) daily. In some embodiments, the maintenance dose is about 5 mg of the compound of Formula (I) daily. In some embodiments, the maintenance dose is about 6 mg of the compound of Formula (I) daily. In some embodiments, the maintenance dose is about 7 mg of the compound of Formula (I) daily. In some embodiments, the maintenance dose is about 8 mg of the compound of Formula (I) daily. In some embodiments, the maintenance dose is about 9 mg of the compound of Formula (I) daily. In some embodiments, the maintenance dose is about 10 mg of the compound of Formula (I) daily. In some embodiments, the maintenance dose is about 11 mg of the compound of Formula (I) daily. In some embodiments, the maintenance dose is about 12 mg of the compound of Formula (I) daily. In some embodiments, the maintenance dose is about 13 mg of the compound of Formula (I) daily. In some embodiments, the maintenance dose is about 14 mg of the compound of Formula (I) daily. In some embodiments, the maintenance dose is about 15 mg of the compound of Formula (I) daily. In some embodiments, the maintenance dose is about 16 mg of the compound of Formula (I) daily. In some embodiments, the maintenance dose is about 17 mg of the compound of Formula (I) daily. In some embodiments, the maintenance dose is about 18 mg of the compound of Formula (I) daily. In some embodiments, the maintenance dose is about 19 mg of the compound of Formula (I) daily. In some embodiments, the maintenance dose is about 20 mg of the compound of Formula (I) daily. In some embodiments, the maintenance dose is about 22 mg of the compound of Formula (I) daily. In some embodiments, the maintenance dose is about 24 mg of the compound of Formula (I) daily. In some embodiments, the maintenance dose is about 26 mg of the compound of Formula (I) daily. In some embodiments, the maintenance dose is about 28 mg of the compound of Formula (I) daily. In some embodiments, the maintenance dose is about 30 mg of the compound of Formula (I) daily. In some embodiments, the maintenance dose is about 32 mg of the compound of Formula (I) daily. In some embodiments, the maintenance dose is about 34 mg of the compound of Formula (I) daily. In some embodiments, the maintenance dose is about 36 mg of the compound of Formula (I) daily. In some embodiments, the maintenance dose is about 38 mg of the compound of Formula (I) daily. In some embodiments, the maintenance dose is about 40 mg of the compound of Formula (I) daily. In some embodiments, the maintenance dose is about 42 mg of the compound of Formula (I) daily. In some embodiments, the maintenance dose is about 44 mg of the compound of Formula (I) daily. In some embodiments, the maintenance dose is about 46 mg of the compound of Formula (I) daily. In some embodiments, the maintenance dose is about 48 mg of the compound of Formula (I) daily. In some embodiments, the maintenance dose is about 50 mg of the compound of Formula (I) daily.

In some embodiments, administering a maintenance dose may include administering about 2 mg to about 150 mg of the compound of Formula (I) once a week to a patient in need thereof. In some embodiments, the maintenance dose is about 2 mg to about 100 mg of the compound of Formula (I) once a week. In some embodiments, the maintenance dose is about 5 mg to about 90 mg of the compound of Formula (I) once a week. In some embodiments, the maintenance dose is about 10 mg to about 80 mg of the compound of Formula (I) once a week. In some embodiments, the maintenance dose is about 10 mg to about 70 mg of the compound of Formula (I) once a week. In some embodiments, the maintenance dose is about 10 mg to about 60 mg of the compound of Formula (I) once a week. In some embodiments, the maintenance dose is about 10 mg to about 50 mg of the compound of Formula (I) once a week. In some embodiments, the maintenance dose is about 10 mg to about 40 mg of the compound of Formula (I) once a week. In some embodiments, the maintenance dose is about 10 mg to about 30 mg of the compound of Formula (I) once a week. In some embodiments, the maintenance dose is about 10 mg to about 20 mg of the compound of Formula (I) once a week. In some embodiments, the maintenance dose is about 2 mg of the compound of Formula (I) once a week. In some embodiments, the maintenance dose is about 3 mg of the compound of Formula (I) once a week. In some embodiments, the maintenance dose is about 4 mg of the compound of Formula (I) once a week. In some embodiments, the maintenance dose is about 5 mg of the compound of Formula (I) once a week. In some embodiments, the maintenance dose is about 6 mg of the compound of Formula (I) once a week. In some embodiments, the maintenance dose is about 7 mg of the compound of Formula (I) once a week. In some embodiments, the maintenance dose is about 8 mg of the compound of Formula (I) once a week. In some embodiments, the maintenance dose is about 9 mg of the compound of Formula (I) once a week. In some embodiments, the maintenance dose is about 10 mg of the compound of Formula (I) once a week. In some embodiments, the maintenance dose is about 11 mg of the compound of Formula (I) once a week. In some embodiments, the maintenance dose is about 12 mg of the compound of Formula (I) once a week. In some embodiments, the maintenance dose is about 13 mg of the compound of Formula (I) once a week. In some embodiments, the maintenance dose is about 14 mg of the compound of Formula (I) once a week. In some embodiments, the maintenance dose is about 15 mg of the compound of Formula (I) once a week. In some embodiments, the maintenance dose is about 16 mg of the compound of Formula (I) once a week. In some embodiments, the maintenance dose is about 17 mg of the compound of Formula (I) once a week. In some embodiments, the maintenance dose is about 18 mg of the compound of Formula (I) once a week. In some embodiments, the maintenance dose is about 19 mg of the compound of Formula (I) once a week. In some embodiments, the maintenance dose is about 20 mg of the compound of Formula (I) once a week. In some embodiments, the maintenance dose is about 22 mg of the compound of Formula (I) once a week. In some embodiments, the maintenance dose is about 24 mg of the compound of Formula (I) once a week. In some embodiments, the maintenance dose is about 26 mg of the compound of Formula (I) once a week. In some embodiments, the maintenance dose is about 28 mg of the compound of Formula (I) once a week. In some embodiments, the maintenance dose is about 30 mg of the compound of Formula (I) once a week. In some embodiments, the maintenance dose is about 32 mg of the compound of Formula (I) once a week. In some embodiments, the maintenance dose is about 34 mg of the compound of Formula (I) once a week. In some embodiments, the maintenance dose is about 36 mg of the compound of Formula (I) once a week. In some embodiments, the maintenance dose is about 38 mg of the compound of Formula (I) once a week. In some embodiments, the maintenance dose is about 40 mg of the compound of Formula (I) once a week. In some embodiments, the maintenance dose is about 42 mg of the compound of Formula (I) once a week. In some embodiments, the maintenance dose is about 44 mg of the compound of Formula (I) once a week. In some embodiments, the maintenance dose is about 46 mg of the compound of Formula (I) once a week. In some embodiments, the maintenance dose is about 48 mg of the compound of Formula (I) once a week. In some embodiments, the maintenance dose is about 50 mg of the compound of Formula (I) once a week.

In some embodiments, administering a maintenance dose may include administering about 2 mg to about 150 mg of the compound of Formula (I) twice a week to a patient in need thereof. In some embodiments, the maintenance dose is about 2 mg to about 100 mg of the compound of Formula (I) twice a week. In some embodiments, the maintenance dose is about 5 mg to about 90 mg of the compound of Formula (I) twice a week. In some embodiments, the maintenance dose is about 10 mg to about 80 mg of the compound of Formula (I) twice a week. In some embodiments, the maintenance dose is about 10 mg to about 70 mg of the compound of Formula (I) twice a week. In some embodiments, the maintenance dose is about 10 mg to about 60 mg of the compound of Formula (I) twice a week. In some embodiments, the maintenance dose is about 10 mg to about 50 mg of the compound of Formula (I) twice a week. In some embodiments, the maintenance dose is about 10 mg to about 40 mg of the compound of Formula (I) twice a week. In some embodiments, the maintenance dose is about 10 mg to about 30 mg of the compound of Formula (I) twice a week. In some embodiments, the maintenance dose is about 10 mg to about 20 mg of the compound of Formula (I) twice a week. In some embodiments, the maintenance dose is about 2 mg of the compound of Formula (I) twice a week. In some embodiments, the maintenance dose is about 3 mg of the compound of Formula (I) twice a week. In some embodiments, the maintenance dose is about 4 mg of the compound of Formula (I) twice a week. In some embodiments, the maintenance dose is about 5 mg of the compound of Formula (I) twice a week. In some embodiments, the maintenance dose is about 6 mg of the compound of Formula (I) twice a week. In some embodiments, the maintenance dose is about 7 mg of the compound of Formula (I) twice a week. In some embodiments, the maintenance dose is about 8 mg of the compound of Formula (I) twice a week. In some embodiments, the maintenance dose is about 9 mg of the compound of Formula (I) twice a week. In some embodiments, the maintenance dose is about 10 mg of the compound of Formula (I) twice a week. In some embodiments, the maintenance dose is about 11 mg of the compound of Formula (I) twice a week. In some embodiments, the maintenance dose is about 12 mg of the compound of Formula (I) twice a week. In some embodiments, the maintenance dose is about 13 mg of the compound of Formula (I) twice a week. In some embodiments, the maintenance dose is about 14 mg of the compound of Formula (I) twice a week. In some embodiments, the maintenance dose is about 15 mg of the compound of Formula (I) twice a week. In some embodiments, the maintenance dose is about 16 mg of the compound of Formula (I) twice a week. In some embodiments, the maintenance dose is about 17 mg of the compound of Formula (I) twice a week. In some embodiments, the maintenance dose is about 18 mg of the compound of Formula (I) twice a week. In some embodiments, the maintenance dose is about 19 mg of the compound of Formula (I) twice a week. In some embodiments, the maintenance dose is about 20 mg of the compound of Formula (I) twice a week. In some embodiments, the maintenance dose is about 22 mg of the compound of Formula (I) twice a week. In some embodiments, the maintenance dose is about 24 mg of the compound of Formula (I) twice a week. In some embodiments, the maintenance dose is about 26 mg of the compound of Formula (I) twice a week. In some embodiments, the maintenance dose is about 28 mg of the compound of Formula (I) twice a week. In some embodiments, the maintenance dose is about 30 mg of the compound of Formula (I) twice a week. In some embodiments, the maintenance dose is about 32 mg of the compound of Formula (I) twice a week. In some embodiments, the maintenance dose is about 34 mg of the compound of Formula (I) twice a week. In some embodiments, the maintenance dose is about 36 mg of the compound of Formula (I) twice a week. In some embodiments, the maintenance dose is about 38 mg of the compound of Formula (I) twice a week. In some embodiments, the maintenance dose is about 40 mg of the compound of Formula (I) twice a week. In some embodiments, the maintenance dose is about 42 mg of the compound of Formula (I) twice a week. In some embodiments, the maintenance dose is about 44 mg of the compound of Formula (I) twice a week. In some embodiments, the maintenance dose is about 46 mg of the compound of Formula (I) twice a week. In some embodiments, the maintenance dose is about 48 mg of the compound of Formula (I) twice a week. In some embodiments, the maintenance dose is about 50 mg of the compound of Formula (I) twice a week.

In some embodiments, administering a maintenance dose may include administering about 2 mg to about 150 mg of the compound of Formula (I) three times a week to a patient in need thereof. In some embodiments, the maintenance dose is about 2 mg to about 100 mg of the compound of Formula (I) three times a week. In some embodiments, the maintenance dose is about 5 mg to about 90 mg of the compound of Formula (I) three times a week. In some embodiments, the maintenance dose is about 10 mg to about 80 mg of the compound of Formula (I) three times a week. In some embodiments, the maintenance dose is about 10 mg to about 70 mg of the compound of Formula (I) three times a week. In some embodiments, the maintenance dose is about 10 mg to about 60 mg of the compound of Formula (I) three times a week. In some embodiments, the maintenance dose is about 10 mg to about 50 mg of the compound of Formula (I) three times a week. In some embodiments, the maintenance dose is about 10 mg to about 40 mg of the compound of Formula (I) three times a week. In some embodiments, the maintenance dose is about 10 mg to about 30 mg of the compound of Formula (I) three times a week. In some embodiments, the maintenance dose is about 10 mg to about 20 mg of the compound of Formula (I) three times a week. In some embodiments, the maintenance dose is about 2 mg of the compound of Formula (I) three times a week. In some embodiments, the maintenance dose is about 3 mg of the compound of Formula (I) three times a week. In some embodiments, the maintenance dose is about 4 mg of the compound of Formula (I) three times a week. In some embodiments, the maintenance dose is about 5 mg of the compound of Formula (I) three times a week. In some embodiments, the maintenance dose is about 6 mg of the compound of Formula (I) three times a week. In some embodiments, the maintenance dose is about 7 mg of the compound of Formula (I) three times a week. In some embodiments, the maintenance dose is about 8 mg of the compound of Formula (I) three times a week. In some embodiments, the maintenance dose is about 9 mg of the compound of Formula (I) three times a week. In some embodiments, the maintenance dose is about 10 mg of the compound of Formula (I) three times a week. In some embodiments, the maintenance dose is about 11 mg of the compound of Formula (I) three times a week. In some embodiments, the maintenance dose is about 12 mg of the compound of Formula (I) three times a week. In some embodiments, the maintenance dose is about 13 mg of the compound of Formula (I) three times a week. In some embodiments, the maintenance dose is about 14 mg of the compound of Formula (I) three times a week. In some embodiments, the maintenance dose is about 15 mg of the compound of Formula (I) three times a week. In some embodiments, the maintenance dose is about 16 mg of the compound of Formula (I) three times a week. In some embodiments, the maintenance dose is about 17 mg of the compound of Formula (I) three times a week. In some embodiments, the maintenance dose is about 18 mg of the compound of Formula (I) three times a week. In some embodiments, the maintenance dose is about 19 mg of the compound of Formula (I) three times a week. In some embodiments, the maintenance dose is about 20 mg of the compound of Formula (I) three times a week. In some embodiments, the maintenance dose is about 22 mg of the compound of Formula (I) three times a week. In some embodiments, the maintenance dose is about 24 mg of the compound of Formula (I) three times a week. In some embodiments, the maintenance dose is about 26 mg of the compound of Formula (I) three times a week. In some embodiments, the maintenance dose is about 28 mg of the compound of Formula (I) three times a week. In some embodiments, the maintenance dose is about 30 mg of the compound of Formula (I) three times a week. In some embodiments, the maintenance dose is about 32 mg of the compound of Formula (I) three times a week. In some embodiments, the maintenance dose is about 34 mg of the compound of Formula (I) three times a week. In some embodiments, the maintenance dose is about 36 mg of the compound of Formula (I) three times a week. In some embodiments, the maintenance dose is about 38 mg of the compound of Formula (I) three times a week. In some embodiments, the maintenance dose is about 40 mg of the compound of Formula (I) three times a week. In some embodiments, the maintenance dose is about 42 mg of the compound of Formula (I) three times a week. In some embodiments, the maintenance dose is about 44 mg of the compound of Formula (I) three times a week. In some embodiments, the maintenance dose is about 46 mg of the compound of Formula (I) three times a week. In some embodiments, the maintenance dose is about 48 mg of the compound of Formula (I) three times a week. In some embodiments, the maintenance dose is about 50 mg of the compound of Formula (I) three times a week.

In some embodiments, such a disclosed method may include, administering a loading dose of 30 mg daily for 5 days, followed by a maintenance dose of 30 mg twice a week, of the compound of Formula (I) to a patient in need thereof.

In some embodiments, such a disclosed method may include, administering a loading dose of 30 mg daily for 3 days, followed by a maintenance dose of 10 mg daily, of the compound of Formula (I) to a patient in need thereof.

In some embodiments, such a disclosed method may include, administering a loading dose of 20 mg daily for 3 days, followed by a maintenance dose of 6 mg daily, of the compound of Formula (I) to a patient in need thereof.

In some embodiments, such a disclosed method may include, administering a dose of 30 mg twice a week, of the compound of Formula (I) to a patient in need thereof.

In some embodiments, administration of the compound of Formula (I), may be effected by any appropriate route including, but not limited to, oral routes, intravenous routes, intramuscular routes, subcutaneous, intratumoral injection, intraarticular injection, and direct absorption through mucous membrane tissues. In some embodiments, administration of the compound of Formula (I), may be effected by oral routes. In some embodiments, administration of the compound of Formula (I), may be effected by intraarticular injection. In some embodiments, administration of the compound of Formula (I), may be effected by intratumoral injection.

In some embodiments, the most suitable administration in any given case will depend on the nature and severity of the condition being treated. The dosages can be conveniently presented in unit dosage form and prepared by any of the methods well-known in the pharmaceutical arts.

In some embodiments, provided herein is a method of treating graft versus host disease (GVHD), comprising administering to the patient a therapeutically effective amount of the formulation comprising the compound represented by Formula (I). In some embodiments, the graft versus host disease (GVHD) is chronic graft versus host disease (cGVHD). In some embodiments, the graft versus host disease (GVHD) is acute graft versus host disease (aGVHD).

In some embodiments, provided herein is a method of treating a neurodegenerative disease, comprising administering to the patient a therapeutically effective amount of the formulation comprising the compound represented by Formula (I). In some embodiments, the neurodegenerative disease is selected from the group consisting of Parkinson's disease (PD), Alzheimer's Disease (AD), frontotemporal dementia (FTD), amyotrophic lateral sclerosis (ALS), mild cognitive impairment, and Huntington's Disease (HD).

In some embodiments, provided herein is a method of treating a cancer selected from the group consisting of solid tumors, acute myeloid leukemia (AML), relapsed/refractory acute myeloid leukemia (AML), relapsed acute myeloid leukemia (AML), refractory acute myeloid leukemia (AML), myelodysplastic syndrome, acute lymphocytic leukemia (ALL), chronic lymphocytic leukemia (CLL), pancreatic ductal adenocarcinoma (PDAC), ovarian cancer, non-small cell lung cancer (NSCLC), prostate cancer, osteosarcoma, breast cancer, colon cancer, and glioblastoma, comprising administering to the patient a therapeutically effective amount of the formulation comprising the compound represented by Formula (I). In some embodiments, the solid tumor is selected from the group consisting of ovarian cancer, pancreatic cancer, prostate cancer, lung cancer, breast cancer, kidney cancer, liver cancer, cervical cancer, bone metastatic cancer, papillary thyroid cancer, non-small cell lung cancer, colon cancer, and gastrointestinal stromal tumor.

In some embodiments, provided herein is a method of treating tumors known to have expression of colony-stimulating factor 1 receptor (CSF-1R) or its ligands, colony stimulating factor-1 (CSF-1), or interleukin (IL)-34 (IL-34), comprising administering to the patient a therapeutically effective amount of the formulation comprising the compound represented by Formula (I).

In some embodiments, provided herein is a method of treating a disease or condition selected from the group consisting of metabolic diseases, rheumatoid arthritis, osteoarthritis, multiple sclerosis, autoimmune nephritis, lupus, Crohn's disease, asthma, chronic obstructive pulmonary diseases, osteoporosis, hypereosinophilic syndromes, mastocytosis, and histiocytosis, comprising administering to the patient a therapeutically effective amount of the formulation comprising the compound represented by Formula (I).

The disclosure contemplates administration of the formulation comprising the compound represented by Formula (I), to a patient in need thereof prior (neo-adjuvant) or after (adjuvant) surgery (e.g., surgical treatment of TGCT). In some embodiments, the formulation comprising the compound represented by Formula (I), is administered as an adjuvant. In some embodiments, the formulation comprising the compound represented by Formula (I), is administered as a neo-adjuvant. In some embodiments, the formulation comprising the compound represented by Formula (I), is administered as a neo-adjuvant and an adjuvant. In some embodiments, the formulation comprising the compound represented by Formula (I), is administered to a patient in need thereof as a neo-adjuvant for a period of 1 month to 6 months, followed by administration of the formulation comprising the compound represented by Formula (I), as an adjuvant for a period from 1 day to 100 years. In some embodiments, administration of the formulation comprising the compound represented by Formula (I), is administered to a patient in need thereof as a neo-adjuvant for a period of 1 month to 6 months, followed by administration of the formulation comprising the compound represented by Formula (I), as an adjuvant for a period from 1 day to 5 years. In some embodiments, administration of the formulation comprising the compound represented by Formula (I), is administered to a patient in need thereof as a neo-adjuvant for a period of 3 months to 6 months, followed by administration of the formulation comprising the compound represented by Formula (I), as an adjuvant for a period from 1 day to 5 years. In some embodiments, no administration of the formulation comprising the compound represented by Formula (I), occurs prior to surgery. In some embodiments, the formulation comprising the compound represented by Formula (I), is administered as an adjuvant for a period from 1 day to 100 years. In some embodiments, the formulation comprising the compound represented by Formula (I), is administered as an adjuvant for a period from 1 day to 5 years.

In some embodiments, provided herein is a method of treating a disease or condition of any other aspects of the disclosure, further comprising administering to the patient a therapeutically effective amount of one or more additional therapeutic agents. In some embodiments, the disease or condition is graft versus host disease (GVHD). In some embodiments, the disease or condition is chronic graft versus host disease (cGVHD). In some embodiments, the disease or condition is acute graft versus host disease (aGVHD). In some embodiments, the one or more additional therapeutic agents is selected from the group consisting of methoxsalen, abatacept, everolimus, alemtuzumab, antithymocyte globulin, autologous serum eye drops, azathioprine, belumosudil, bortezomib, mycophenolate mofetil, cyclophosphamide, cyclosporine, extracorporeal photopheresis, etanercept, imatinib mesylate, ibrutinib, interleukin-2, infliximab, ruxolitinib, methotrexate, muromab-CD3, pentostatin, denileukin diftitox, prednisone, prednisolone, tacrolimus, psoralen with ultraviolet A light, sirolimus, rituximab, methylprednisolone, budesonide, thalidomide, halofuginone, and hydroxychloroquine. In some embodiments, the one or more additional therapeutic agents is belumosudil.

In some embodiments, provided herein is a method of treating a disease or condition of any other aspects of the disclosure, further comprising administering to the patient a therapeutically effective amount of one or more additional therapeutic agents. In some embodiments, the disease or condition is a cancer selected from the group consisting of solid tumors, acute myeloid leukemia (AML), relapsed/refractory acute myeloid leukemia (AML), relapsed acute myeloid leukemia (AML), refractory acute myeloid leukemia (AML), myelodysplastic syndrome, acute lymphocytic leukemia (ALL), chronic lymphocytic leukemia (CLL), pancreatic ductal adenocarcinoma (PDAC), ovarian cancer, non-small cell lung cancer (NSCLC), prostate cancer, osteosarcoma, breast cancer, colon cancer, and glioblastoma. In some embodiments, the one or more additional therapeutic agents is an immunomodulatory therapeutic. In some embodiments, the one or more additional therapeutic agents is a chemotherapeutic agent. In some embodiments, the one or more additional therapeutic agents is an immunomodulatory therapeutic and a chemotherapeutic agent.

The compound represented by Formula (I), can be administered in combination with one or more additional therapeutic agents to treat a disorder described herein, such as cancer. For example, provided in the present disclosure is a formulation comprising the compound represented by Formula (I), one or more additional therapeutic agents, and a pharmaceutically acceptable excipient. In some embodiments, the compound represented by Formula (I), and one additional therapeutic agent is administered. In some embodiments, the compound represented by Formula (I), and two additional therapeutic agents are administered. In some embodiments, the compound represented by Formula (I), and three additional therapeutic agents are administered. Combination therapy can be achieved by administering two or more therapeutic agents, each of which is formulated and administered separately. For example, the compound represented by Formula (I), and an additional therapeutic agent can be formulated and administered separately. Combination therapy can also be achieved by administering two or more therapeutic agents in a single formulation, for example a formulation comprising the compound represented by Formula (I), as one therapeutic agent and one or more additional therapeutic agents. For example, the compound represented by Formula (I), and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases, even longer intervals are possible. While in many cases it is desirable that the two or more agents used in a combination therapy be present in within the patient's body at the same time, this need not be so.

“Combination therapy” (or “co-therapy”) includes the administration of the compound represented by Formula (I), and at least a second agent, e.g., an anti-PD1 or anti-PD-L1 therapeutic, an anti-PD1 or anti-PD-L1 antibody, an immune-check point inhibitor, or a chemotherapeutic agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination can be carried out over a defined time period (usually minutes, hours, days or weeks depending upon the combination selected) or until disease progression. Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single tablet or capsule having a fixed ratio of each therapeutic agent or in multiple, single unit doses for each of the therapeutic agents. Sequential or substantially simultaneous administration of each therapeutic agent can be effected by any appropriate route including, but not limited to, oral routes, intravenous routes, intramuscular routes, subcutaneous, intratumoral injection, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally, or all therapeutic agents may be administered by intravenous injection. The sequence in which the therapeutic agents are administered is not narrowly critical.

Combination therapy also can embrace the administration of the therapeutic agents as described above in further combination with other biologically active ingredients and non-drug therapies, including, but not limited to, radiation therapy. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The components of the combination may be administered to a patient simultaneously, sequentially or in combination. It will be appreciated that when using a formulation or method the components may be in the same pharmaceutically acceptable excipient and therefore administered simultaneously. They may be in separate pharmaceutical excipients such as conventional oral dosage forms which are taken simultaneously. The term “combination” further refers to the case where the compounds are provided in separate dosage forms and are administered sequentially.

Although not wishing to be bound by theory, it is thought that the administration of the compound represented by Formula (I), in accordance with the methods described herein, in combination with one or more anti-PD1 or anti-PD-L1 therapeutics may provide additive effects in significantly inhibiting primary tumor growth and modulating the immune system into an antitumoral state, which can be beneficial in the treatment of disorders associated with the proliferation, survival, or biological action of macrophages, including the treatment of TGCT. Examples of anti-PD1 or anti-PD-L1 therapeutics that may be administered in combination with CSF-1R inhibitors described herein include, but are not limited to, nivolumab, pidilizumab, cemiplimab, tislelizumab, AMP-224, AMP-514, and pembrolizumab.

The compound represented by Formula (I), can be used in combination with other immunomodulatory agents including but not limited to anti-PD-L1 therapeutics including atezolizumab, durvalumab, BMS-936559, and avelumab, anti-TIM3 therapeutics including TSR-022 and MBG453, anti-LAG3 therapeutics including relatlimab, LAG525, and TSR-033, CD40 agonist therapeutics including SGN-40, CP-870,893 and R07009789, anti-CD47 therapeutics including Hu5F9-G4, anti-CD20 therapeutics, anti-CD38 therapeutics, or other immunomodulatory therapeutics including thalidomide, lenalidomide, pomalidomide, mezigdomide, prednisone, and dexamethasone.

Sarcomas comprise a diverse group of malignancies including more than fifty subtypes of bone and soft tissue origin. In some embodiments, a method of treating a cancer comprises administering to a patient with locally advanced and metastatic high-grade sarcoma the compound represented by Formula (I), in combination with avelumab. In some embodiments, a method of treating a cancer comprises administering to a patient with locally advanced sarcoma the compound represented by Formula (I), in combination with avelumab. In some embodiments, a method of treating a cancer comprises administering to a patient with metastatic high-grade sarcoma the compound represented by Formula (I), in combination with avelumab. In some embodiments, a method of treating a cancer comprises administering to a patient with advanced metastatic sarcoma the compound represented by Formula (I), in combination with avelumab. In some embodiments, a method of treating a cancer comprises administering to a patient with undifferentiated pleomorphic sarcoma (UPS) the compound represented by Formula (I), in combination with avelumab. In some embodiments, a method of treating a cancer comprises administering to a patient with myxofibrosarcoma (MFS) the compound represented by Formula (I), in combination with avelumab. In some embodiments, a method of treating a cancer comprises administering to a patient with leiomyosarcoma (LMS) the compound represented by Formula (I), in combination with avelumab. In some embodiments, a method of treating a cancer comprises administering to a patient with dedifferentiated liposarcoma (DDLPS) the compound represented by Formula (I), in combination with avelumab.

The compound represented by Formula (I), can also be used in combination with one or more chemotherapeutic agents including but not limited to anti-tubulin agents (e.g., paclitaxel, paclitaxel protein-bound particles for injectable suspension, eribulin, abraxane, docetaxel, ixabepilone, vincristine or vinorelbine), LHRH antagonists including but not limited to leuprolide, goserelin, triptorelin, or histrelin, anti-androgen agents including but not limited to abiraterone, flutamide, bicalutamide, nilutamide, cyproterone acetate, enzalutamide, and apalutamide, anti-estrogen agents including but not limited to tamoxifen, fulvestrant, anastrozole, letrozole, and exemestane, DNA-alkylating agents (including cisplatin, carboplatin, oxaliplatin, cyclophosphamide, ifosfamide, and temozolomide), DNA intercalating agents (including doxorubicin, pegylated liposomal doxorubicin, daunorubicin, idarubicin, and epirubicin), 5-fluorouracil, capecitabine, cytarabine, decitabine, 5-aza cytidine, gemcitabine methotrexate, bortezomib, and carfilzomib.

The compound represented by Formula (I), can also be used in combination with targeted therapeutics including kinase inhibitors erlotinib, gefitinib, lapatanib, everolimus, temsirolimus, abemaciclib, LEE011, palbociclib, ribociclib, crizotinib, cabozantinib, sunitinib, pazopanib, sorafenib, regorafenib, axitinib, dasatinib, imatinib, nilotinib, vemurafenib, dabrafenib, trametinib, cobimetinib, binimetinib, idelalisib, quizartinib, avapritinib, BLU-667, BLU-263, Loxo 292, larotrectinib, and quizartinib, anti-estrogen agents including but not limited to tamoxifen, fulvestrant, anastrozole, letrozole, and exemestane, anti-androgen agents including but not limited to abiraterone acetate, enzalutamide, nilutamide, bicalutamide, flutamide, cyproterone acetate, steroid agents including but not limited to prednisone and dexamethasone, PARP inhibitors including but not limited to neraparib, olaparib, and rucaparib, topoisomerase I inhibitors including but not limited to irinotecan, camptothecin, and topotecan, topoisomerase II inhibitors including but not limited to etoposide, etoposide phosphate, and mitoxantrone, Histone Deacetylase (HDAC) inhibitors including but not limited to vorinostat, romidepsin, panobinostat, valproic acid, and belinostat, DNA methylation inhibitors including but not limited to DZNeP and 5-aza-2′-deoxycytidine, proteasome inhibitors including but not limited to bortezomib and carfilzomib, thalidomide, lenalidomide, pomalidomide, biological agents including but not limited to trastuzumab, ado-trastuzumab, pertuzumab, cetuximab, panitumumab, ipilimumab, tremelimumab, vaccines including but not limited to sipuleucel-T, and radiotherapy.

The represented by Formula (I), can also be used in combination with anti-angiogenic agents including AMG386, bevacizumab and aflibercept, and antibody-drug-conjugates (ADCs) including but not limited to gemtuzumab ozogamicin, brentuximab vedotin, trastuzumab emtansine, inotuzumab ozogamicin, polatuzumab vedotin, enfortumab vedotin, trastuzumab deruxtecan, sacituzumab govitecan, belantamab mafodotin, moxetumomab pasudotox, loncastuximab tesirine, wherein the payload contained in the ADCs includes but is not limited to a derivative of camptothecin, a pyrrolobenzodiazepine dimer (PBD), an indolinobenzodiazepine dimer (IGN), DM1, DM4, MMAE, or MMAF.

In some embodiments, the additional therapeutic agent is selected from a luteinizing hormone-releasing hormone (LHRH) analog, including goserelin and leuprolide.

In some embodiments, the additional therapeutic agent is selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, AZD2171, batabulin, of atumtunab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, RTA 744, SDX 102, talampanel, atrasentan, XR 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib, PD0325901, AZD-6244, capecitabine, L-Glutamic acid, N-[4-[2-(2-amino-4,7-dihydro-4-oxo-1H-pyrrolo[2,3-d]pyrimidin-5-yl)-ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258), 3-[5-(methylsulfonylpiperadinemethyl)-indolylj-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutanide, nilutamide, megestrol acetate, CP-724714, TAK-165, HKI-272, erlotinib, lapatanib, canertinib, ABX-EGF antibody, erbitux, EKB-569, PKI-166, GW-572016, Ionafarnib, BMS-214662, tipifarnib, amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilumumab, vemurafenib, and mixtures thereof.

In some embodiments of any other aspects of the disclosure, the therapeutically effective amount of the formulation comprising the compound represented by Formula (I), is administered orally.

In some embodiments, provided herein is a method of treating a tenosynovial giant cell tumor, comprising orally administering to the patient a therapeutically effective amount of the formulation comprising the compound represented by Formula (I). In some embodiments, the tenosynovial giant cell tumor is a diffuse-type tenosynovial giant cell tumor. In some embodiments, the tenosynovial giant cell tumor is a localized tenosynovial giant cell tumor.

In some embodiments, provided herein is a method of treating graft versus host disease (GVHD), comprising orally administering to the patient a therapeutically effective amount of the formulation comprising the compound represented by Formula (I). In some embodiments, the graft versus host disease (GVHD) is chronic graft versus host disease (cGVHD). In some embodiments, the graft versus host disease (GVHD) is acute graft versus host disease (aGVHD).

In some embodiments, provided herein is a method of treating a neurodegenerative disease, comprising orally administering to the patient a therapeutically effective amount of the formulation comprising the compound represented by Formula (I). In some embodiments, the neurodegenerative disease is selected from the group consisting of Parkinson's disease (PD), Alzheimer's Disease (AD), frontotemporal dementia (FTD), amyotrophic lateral sclerosis (ALS), mild cognitive impairment, and Huntington's Disease (HD).

In some embodiments, provided herein is a method of treating a cancer selected from the group consisting of solid tumors, acute myeloid leukemia (AML), relapsed/refractory acute myeloid leukemia (AML), relapsed acute myeloid leukemia (AML), refractory acute myeloid leukemia (AML), myelodysplastic syndrome, acute lymphocytic leukemia (ALL), chronic lymphocytic leukemia (CLL), pancreatic ductal adenocarcinoma (PDAC), ovarian cancer, non-small cell lung cancer (NSCLC), prostate cancer, osteosarcoma, breast cancer, colon cancer, and glioblastoma, comprising orally administering to the patient a therapeutically effective amount of the formulation comprising the compound represented by Formula (I). In some embodiments, the solid tumor is selected from the group consisting of ovarian cancer, pancreatic cancer, prostate cancer, lung cancer, breast cancer, kidney cancer, liver cancer, cervical cancer, bone metastatic cancer, papillary thyroid cancer, non-small cell lung cancer, colon cancer, and gastrointestinal stromal tumor.

In some embodiments, provided herein is a method of treating tumors known to have expression of colony-stimulating factor 1 receptor (CSF-1R) or its ligands, colony stimulating factor-1 (CSF-1), or interleukin (IL)-34 (IL-34), comprising orally administering to the patient a therapeutically effective amount of the formulation comprising the compound represented by Formula (I).

In some embodiments, provided herein is a method of treating a disease or condition selected from the group consisting of metabolic diseases, rheumatoid arthritis, osteoarthritis, multiple sclerosis, autoimmune nephritis, lupus, Crohn's disease, asthma, chronic obstructive pulmonary diseases, osteoporosis, hypereosinophilic syndromes, mastocytosis, and histiocytosis, comprising orally administering to the patient a therapeutically effective amount of the formulation comprising the compound represented by Formula (I).

In some embodiments, provided herein is a method of treating a disease or condition of any other aspects of the disclosure, further comprising administering to the patient a therapeutically effective amount of one or more additional therapeutic agents. In some embodiments, the disease or condition is graft versus host disease (GVHD). In some embodiments, the disease or condition is chronic graft versus host disease (cGVHD). In some embodiments, the disease or condition is acute graft versus host disease (aGVHD). In some embodiments, the one or more additional therapeutic agents is selected from the group consisting of methoxsalen, abatacept, everolimus, alemtuzumab, antithymocyte globulin, autologous serum eye drops, azathioprine, belumosudil, bortezomib, mycophenolate mofetil, cyclophosphamide, cyclosporine, extracorporeal photopheresis, etanercept, imatinib mesylate, ibrutinib, interleukin-2, infliximab, ruxolitinib, methotrexate, muromab-CD3, pentostatin, denileukin diftitox, prednisone, prednisolone, tacrolimus, psoralen with ultraviolet A light, sirolimus, rituximab, methylprednisolone, budesonide, thalidomide, halofuginone, and hydroxychloroquine. In some embodiments, the one or more additional therapeutic agents is belumosudil.

In some embodiments, provided herein is a method of treating a disease or condition of any other aspects of the disclosure, further comprising administering to the patient a therapeutically effective amount of one or more additional therapeutic agents. In some embodiments, the disease or condition is a cancer selected from the group consisting of solid tumors, acute myeloid leukemia (AML), relapsed/refractory acute myeloid leukemia (AML), relapsed acute myeloid leukemia (AML), refractory acute myeloid leukemia (AML), myelodysplastic syndrome, acute lymphocytic leukemia (ALL), chronic lymphocytic leukemia (CLL), pancreatic ductal adenocarcinoma (PDAC), ovarian cancer, non-small cell lung cancer (NSCLC), prostate cancer, osteosarcoma, breast cancer, colon cancer, and glioblastoma. In some embodiments, the one or more additional therapeutic agents is an immunomodulatory therapeutic. In some embodiments, the one or more additional therapeutic agents is a chemotherapeutic agent. In some embodiments, the one or more additional therapeutic agents is an immunomodulatory therapeutic and a chemotherapeutic agent.

In some embodiments, provided herein is a use of the formulation comprising the compound represented by Formula (I), in the manufacture of a medicament for the treatment of a tenosynovial giant cell tumor. In some embodiments, the tenosynovial giant cell tumor is a diffuse-type tenosynovial giant cell tumor. In some embodiments, the tenosynovial giant cell tumor is a localized tenosynovial giant cell tumor.

In some embodiments, provided herein is a use of the formulation comprising the compound represented by Formula (I), in the manufacture of a medicament for the treatment of graft versus host disease (GVHD). In some embodiments, the graft versus host disease (GVHD) is chronic graft versus host disease (cGVHD). In some embodiments, the graft versus host disease (GVHD) is acute graft versus host disease (aGVHD).

In some embodiments, provided herein is a use of the formulation comprising the compound represented by Formula (I), in the manufacture of a medicament for the treatment of a neurodegenerative disease. In some embodiments, the neurodegenerative disease is selected from the group consisting of Parkinson's disease (PD), Alzheimer's Disease (AD), frontotemporal dementia (FTD), amyotrophic lateral sclerosis (ALS), mild cognitive impairment, and Huntington's Disease (HD).

In some embodiments, provided herein is a use of the formulation comprising the compound represented by Formula (I), in the manufacture of a medicament for the treatment of a cancer selected from the group consisting of solid tumors, acute myeloid leukemia (AML), relapsed/refractory acute myeloid leukemia (AML), relapsed acute myeloid leukemia (AML), refractory acute myeloid leukemia (AML), myelodysplastic syndrome, acute lymphocytic leukemia (ALL), chronic lymphocytic leukemia (CLL), pancreatic ductal adenocarcinoma (PDAC), ovarian cancer, non-small cell lung cancer (NSCLC), prostate cancer, osteosarcoma, breast cancer, colon cancer, and glioblastoma. In some embodiments, the solid tumor is selected from the group consisting of ovarian cancer, pancreatic cancer, prostate cancer, lung cancer, breast cancer, kidney cancer, liver cancer, cervical cancer, bone metastatic cancer, papillary thyroid cancer, non-small cell lung cancer, colon cancer, and gastrointestinal stromal tumor.

In some embodiments, provided herein is a use of the formulation comprising the compound represented by Formula (I), in the manufacture of a medicament for the treatment of tumors known to have expression of colony-stimulating factor 1 receptor (CSF-1R) or its ligands, colony stimulating factor-1 (CSF-1), or interleukin (IL)-34 (IL-34.

In some embodiments, provided herein is a use of the formulation comprising the compound represented by Formula (I), in the manufacture of a medicament for the treatment of a disease or condition selected from the group consisting of metabolic diseases, rheumatoid arthritis, osteoarthritis, multiple sclerosis, autoimmune nephritis, lupus, Crohn's disease, asthma, chronic obstructive pulmonary diseases, osteoporosis, hypereosinophilic syndromes, mastocytosis, and histiocytosis.

In some aspects of the disclosure, in the use of the compound represented by Formula (I) of the compound represented by Formula (I), in the manufacture of a medicament for the treatment of a disease or condition, the amount of the compound represented by Formula (I) is about 1 mg to about 150 mg. In some embodiments, the amount of the compound represented by Formula (I) is about 2 mg to about 35 mg. In some embodiments, the amount of the anhydrous form of the compound represented by Formula (I) is about 25.0 mg, about 25.1 mg, about 25.2 mg, about 25.3 mg, about 25.4 mg, about 25.5 mg, about 25.6 mg, about 25.7 mg, about 25.8 mg, and about 25.9 mg. In some embodiments, the amount of the anhydrous form of the compound represented by Formula (I) is about 50.0 mg, about 50.1 mg, about 50.2 mg, about 50.3 mg, about 50.4 mg, about 50.5 mg, about 50.6 mg, about 50.7 mg, about 50.8 mg, and about 50.9 mg,

In some aspects of the disclosure, in the use of the anhydrous form of the compound represented by Formula (I) of the anhydrous form of the compound represented by Formula (I), in the manufacture of a medicament for the treatment of a disease or condition, the amount of the anhydrous form of the compound represented by Formula (I) is about 1 mg to about 150 mg. In some embodiments, the amount of the anhydrous form of the compound represented by Formula (I) is about 25.0 mg, about 25.1 mg, about 25.2 mg, about 25.3 mg, about 25.4 mg, about 25.5 mg, about 25.6 mg, about 25.7 mg, about 25.8 mg, and about 25.9 mg. In some embodiments, the amount of the anhydrous form of the compound represented by Formula (I) is about 50.0 mg, about 50.1 mg, about 50.2 mg, about 50.3 mg, about 50.4 mg, about 50.5 mg, about 50.6 mg, about 50.7 mg, about 50.8 mg, and about 50.9 mg.

In some aspects of the disclosure, in the use of the anhydrous form of a crystalline hydrochloride salt (e.g., monohydrochloride salt) of compound represented by Formula (I), in the manufacture of a medicament for the treatment of a disease or condition, the amount of the anhydrous form of the crystalline hydrochloride salt of the compound represented by Formula (I) is about 1 mg to about 150 mg. In some embodiments, the amount of the anhydrous form of the compound represented by Formula (I) is about 27.0 mg, about 27.1 mg, about 27.2 mg, about 27.3 mg, about 27.4 mg, about 27.5 mg, about 27.6 mg, about 27.7 mg, about 27.8 mg, and about 27.9 mg. In some embodiments, the amount of the anhydrous form of the crystalline hydrochloride salt of the compound represented by Formula (I) is about 54.0 mg, about 54.1 mg, about 54.2 mg, about 54.3 mg, about 54.4 mg, about 54.5 mg, about 54.6 mg, about 54.7 mg, about 54.8 mg, and about 54.9 mg.

In some aspects of the disclosure, in the use of a monohydrate form of a crystalline hydrochloride salt (e.g., monohydrate monohydrochloride salt or dihydrate hydrochloride salt) of compound represented by Formula (I), in the manufacture of a medicament for the treatment of a disease or condition, the amount of the monohydrate form of the crystalline hydrochloride salt represented by Formula (I) is about 1 mg to about 150 mg. In some embodiments, the monohydrate form of the crystalline hydrochloride salt of the compound represented by Formula (I) is about 28.0 mg, about 28.1 mg, about 28.2 mg, about 28.3 mg, about 28.4 mg, about 28.5 mg, about 28.6 mg, about 28.7 mg, about 28.8 mg, and about 28.9 mg. In some embodiments, the amount of the anhydrous form of the compound represented by Formula (I) is about 56.0 mg, about 56.1 mg, about 56.2 mg, about 56.3 mg, about 56.4 mg, about 56.5 mg, about 56.6 mg, about 56.7 mg, about 56.8 mg, and about 56.9 mg.

### Kits

Also provided herein are kits comprising one or more containers comprising one or more formulations, dosage forms, or capsules disclosed herein and optionally a notice in the form prescribed by a governmental agency regulating the manufacture, use or sale of pharmaceuticals or biological products, wherein the notice reflects approval by the agency of manufacture, use or sale for human administration for treating a condition, disease, or disorder described herein and/or instruction for use of the kit in treating a condition, disease, or disorder described herein in a subject in need thereof. In some embodiments, the kit comprises a specific amount of the individual doses in a package containing one or more formulations, dosage forms, or capsules disclosed herein. The specified amount of individual doses may contain about 1 to about 100 individual dosages, alternatively about 1 to about 60 individual dosages, alternatively about 10 to about 30 individual dosages, including, about 1, about 2, about 5, about 10, about 15, about 20, about 25, about 30, about 35, about 40, about 45, about 50, about 55, about 60, about 70, about 80, about 100, and include any additional increments thereof, for example, 1, 2, 5, 10 and multiplied factors thereof, (e.g., ×1, ×2, ×2.5, ×5, ×10, ×100, etc.). In some embodiments, the kit comprises the one or more formulations, dosage forms, or capsules disclosed herein in a blister pack. In some embodiments, the kit comprises the one or more formulations, dosage forms, or capsules disclosed herein contained within a bottle or container. For example, the bottle or container may contain about 10, about 25, about 50, about 75, about 100, about 125, about 150, about 175, or about 200 oral dosage forms such as capsules or tablets. In some embodiments, the kit further comprises a desiccant such as, but not limited to, silica (e.g., silica gel) and activated carbon (e.g., activated carbon pouch).

### Examples

The present disclosure is not to be limited in scope by the specific embodiments disclosed in the examples which are intended as illustrations of a few aspects of the disclosure and any embodiments that are functionally equivalent are within the scope of this disclosure. Indeed, various modifications in addition to those shown and described herein will become apparent to those skilled in the art and are intended to fall within the scope of the appended claims.

### Methods and Materials

The X-ray powder diffraction pattern of the acid salt of the crystalline compound of the formula (I) is characterized. Therefore, the X-ray powder diffraction pattern of the salt is collected on a Rigaku Ultima IV powder diffractometer operated in a reflective manner with Cu Kα radiation. The instrument is operated at room temperature by using a D/tex Ultra detector with Cu Kα irradiation (40 kV, 40 mA). The scanning area is from 3° to 450 in the 20 range, and the scanning speed is 20°/min. The diffraction pattern is analyzed by using Jade 5 software with 5.0.37 Materials Data, Inc, published in 2017.

XRPD samples are prepared by placing the samples on a monocrystalline silicon wafer and pressing the sample powder with a glass sheet or an equivalent object to ensure that the surfaces of the samples are flat at an appropriate height. Then a sample support is placed into the Rigaku Ultima IV XRPD instrument, and the X-ray powder diffraction pattern is collected by using the instrument parameters described above. Measurement differences associated with such X-ray powder diffraction analysis results are generated from multiple factors including: (a) errors in the sample preparation (e.g., the sample height), (b) instrument errors, (c) calibration differences, (d) operator errors (including those errors that occur when determining the peak position), and (e) nature of substances (e.g., a preferred orientation error). Calibration errors and sample height errors often cause all peaks to shift in the same direction. Generally, the calibration factor will align the determined peak position with the expected peak position within the range of the expected 2θ value±0.2°. The angle 2θ values (°) and intensity values (% of the highest peak value) of the polymorphic forms obtained by the embodiments of the present invention have been listed in Tables 1 to 13.

The experimental method for characterizing the acid salt of the crystalline compound of the formula (I) by using differential scanning calorimetry (DSC) comprises the steps of putting a small amount of powder of the acid salt of the crystalline compound of the formula (I) in an aluminum disk which is in coordination with the instrument and can be covered, covering with the aluminum disk after loading the samples, and then sending to the instrument for testing. The model of all the instruments used in the differential scanning calorimetry in the present invention is TA Q2000, the scanning parameters are set to that a nitrogen atmosphere is used, and the heating speed is 10° C./min.

The experimental method for characterizing the acid salt of the crystalline compound of the formula (I) by using thermogravimetric analysis (TGA) comprises the steps of putting a small amount of powder of the acid salt of the crystalline compound of the formula (I) in an aluminum disk in coordination with the instrument, and then sending to the instrument for testing after loading the samples. The model of all the instruments used in the thermogravimetric analysis in the present invention is TA Q500, the scanning parameters are set to that a nitrogen atmosphere is used, and the heating speed is 10° C./min.

The experimental method for characterizing the acid salt of the crystalline compound of the formula (I) by using dynamic vapor sorption (DVS) comprises the steps of putting a small amount of powder of the acid salt of the crystalline compound of the formula (I) in a precision sample tray in coordination with the instrument, and then sending to the instrument for testing after loading the samples. The model of all the instruments used in the dynamic vapor sorption in the present invention is DVS Intrinsic, the experimental parameters are set to that nitrogen is used as the carrier gas, the constant temperature is set to 25° C., the mass percent change rate per unit time (dm/dt) is equal to 0.01%/min as the determination criteria to achieve balance, the program humidity change cycle is set to that the initial relative humidity is 0%, the relative humidity at the end point is 90%, cycle is set to twice, and every 10% R.H. changes a step.

The reagents in the embodiments of the present invention are known and commercially available, or can be synthesized by using or in accordance with methods known in the art, and API raw materials are prepared according to the patent WO2018214867A1.

Unless otherwise specified, all reactions of the present invention are carried out under continuous magnetic stirring and a dry nitrogen or argon atmosphere, the solvent is a dry solvent, and the temperature unit is degrees Celsius (° C.).

Unless otherwise specified, all the crystalline forms described in the present invention may be crystal-free forms or crystal-containing forms, such as the crystal-containing forms, preferably, each subcrystal contains 1, 2, 3, 4 or 5 parts of subcrystal water, and more preferably, each subcrystal contains 1 or 2 parts of subcrystal water.

### Example 1. Preparation of Hydrochloride Crystalline Form I

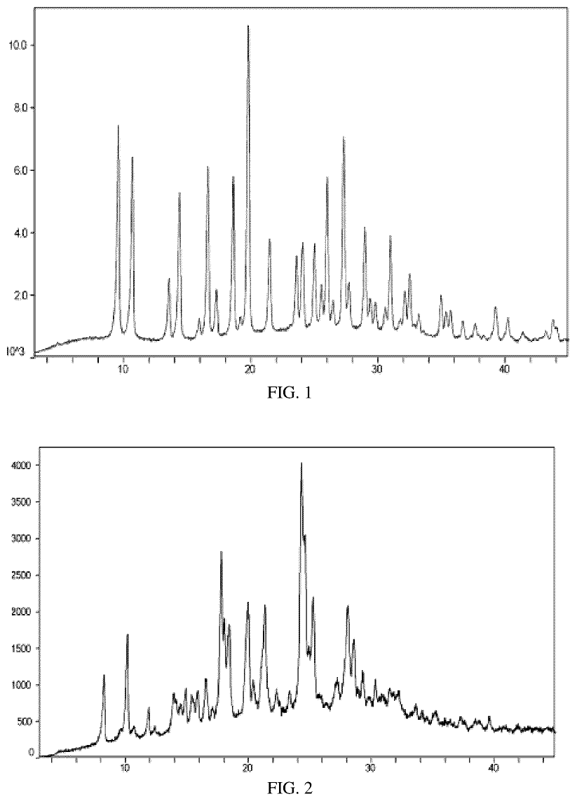

About 10 mg of the compound of the formula (I) was dissolved in 0.5 mL of methyl tertiary butyl ether, 0.1 mL of methyl tertiary butyl ether containing 2.35 μL of concentrated hydrochloric acid was added, stirring was performed at room temperature for 3 days, filtering was performed, a filter cake was dried in an oven at 50° C. for XRPD analysis, and the X-ray powder diffraction pattern was shown in FIG. 1.

### Example 2. Preparation of Hydrochloride Crystalline Form II

About 10 mg of the compound of the formula (I) was dissolved in 0.5 mL of ethyl acetate, 0.1 mL of ethyl acetate containing 2.35 μL of concentrated hydrochloric acid was added, stirring was performed at room temperature for 3 days, filtering was performed, a filter cake was dried in an oven at 50° C. for XRPD analysis, and the X-ray powder diffraction pattern was shown in FIG. 2.

### Example 3. Preparation of Hydrochloride Crystalline Form III

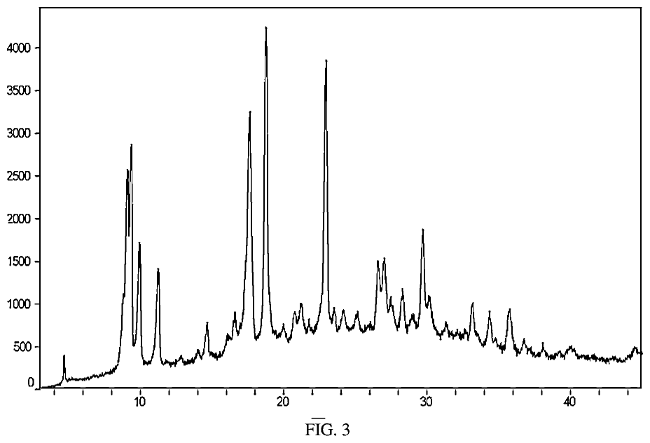

About 10 mg of the compound of the formula (I) was dissolved in 0.5 mL of acetone, 0.1 mL of acetone containing 2.35 μL of concentrated hydrochloric acid was added, stirring was performed at room temperature for 3 days, filtering was performed, a filter cake was dried in an oven at 50° C. for XRPD analysis, and the X-ray powder diffraction pattern was shown in FIG. 3.

### Example 4. Preparation of Hydrochloride Crystalline Form I

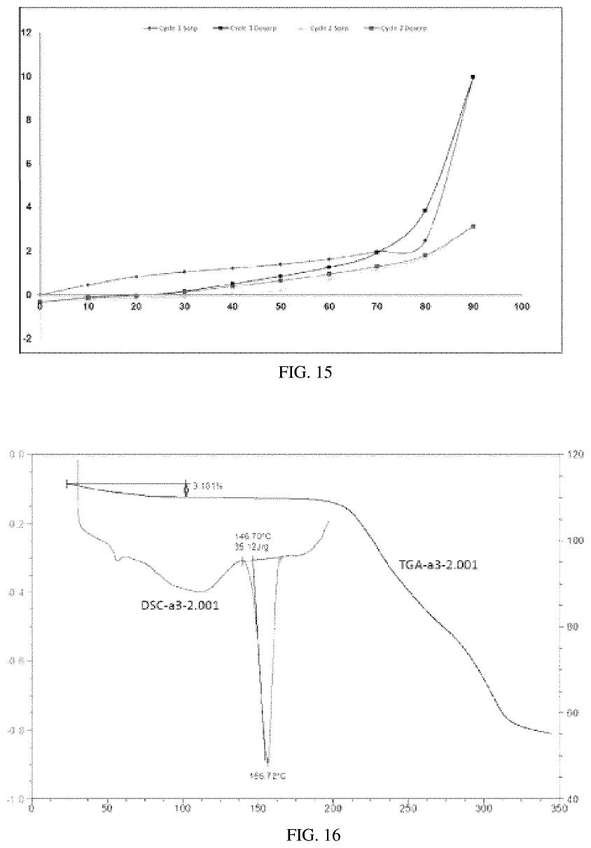

About 50 mg of the compound of the formula (I) was dissolved in 1.2 mL of methyl tertiary butyl ether, 1.2 mL of methyl tertiary butyl ether containing 11.7 μL of concentrated hydrochloric acid was added, stirring was performed at room temperature for 7 days, filtering was performed, a filter cake was dried in an oven at 50° C. for XRPD, DSC, TGA and DVS analysis, the X-ray powder diffraction pattern was consistent with that shown in FIG. 1, and the DSC, TGA and DVS analysis were shown in FIG. 14-15.

### Example 5. Preparation of Sulfate Crystalline Form I

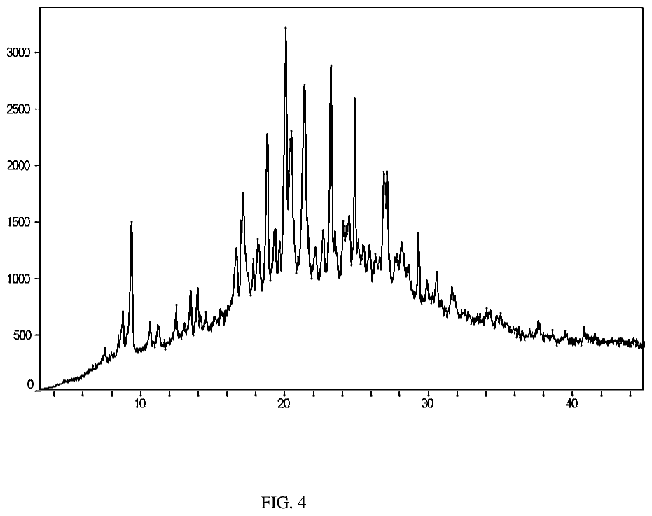

About 10 mg of the compound of the formula (I) was dissolved in 0.5 mL of methyl tertiary butyl ether, 0.1 mL of methyl tertiary butyl ether containing 4.67 μL of concentrated hydrochloric acid was added, stirring was performed at room temperature for 3 days, filtering was performed, a filter cake was dried in an oven at 50° C. for XRPD analysis, and the X-ray powder diffraction pattern was shown in FIG. 4.

### Example 6. Preparation of Phosphate Crystalline Form I

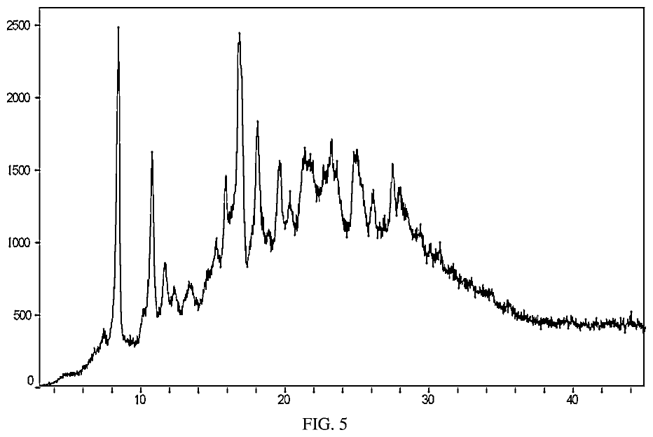

About 10 mg of the compound of the formula (I) was dissolved in 0.5 mL of methanol, 0.1 mL of methanol containing 2.74 μL of concentrated phosphoric acid was added, stirring was performed, standing was performed at −20° C. for 3 days, filtering was performed, a filter cake was dried in an oven at 50° C. for XRPD analysis, and the X-ray powder diffraction pattern was shown in FIG. 5.

### Example 7. Preparation of Phosphate Crystalline Form II

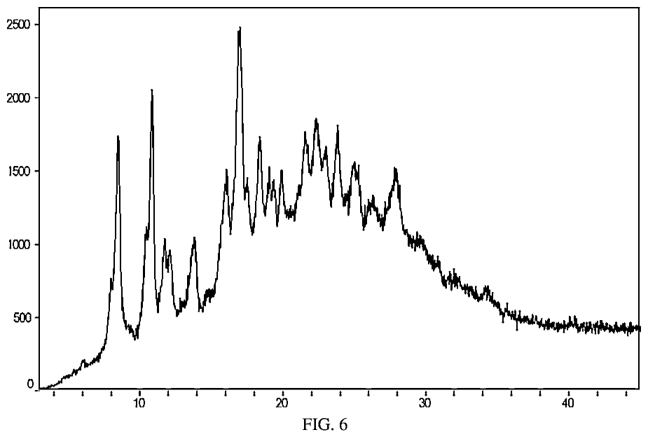

About 10 mg of the compound of the formula (I) was dissolved in 0.5 mL of ethyl acetate, 0.1 mL of ethyl acetate containing 2.74 μL of concentrated phosphoric acid was added, stirring was performed, standing was performed at −20° C. for 3 days, filtering was performed, a filter cake was dried in an oven at 50° C. for XRPD analysis, and the X-ray powder diffraction pattern was shown in FIG. 6.

### Example 8. Preparation of Phosphate Crystalline Form III

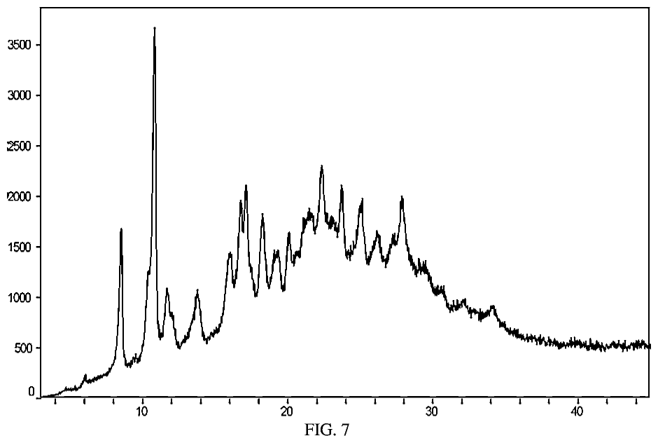

About 10 mg of the compound of the formula (I) was dissolved in 0.5 mL of 96% ethanol, 0.1 mL of 96% ethanol containing 2.74 μL of concentrated phosphoric acid was added, stirring was performed, standing was performed at −20° C. for 3 days, filtering was performed, a filter cake was dried in an oven at 50° C. for XRPD analysis, and the X-ray powder diffraction pattern was shown in FIG. 7.

### Example 9. Preparation of Phosphate Crystalline Form I

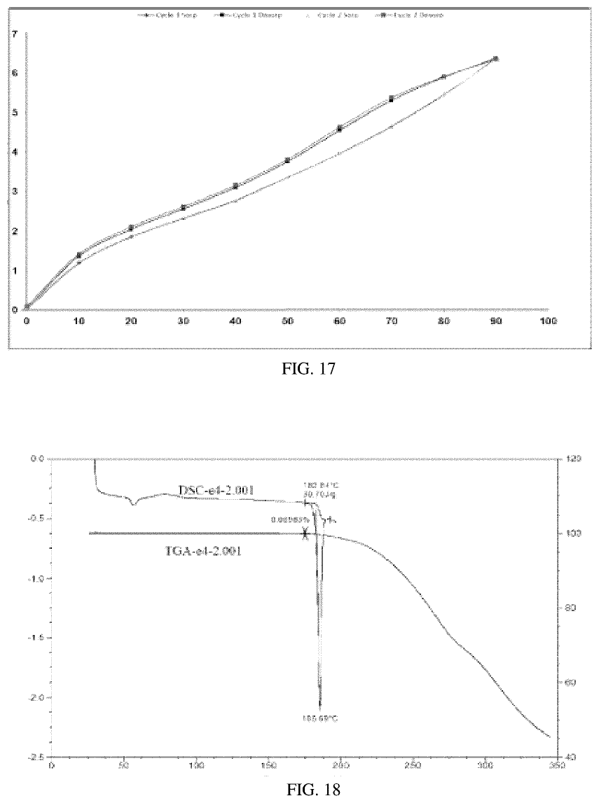

About 50 mg of the compound of the formula (I) was dissolved in 1.2 mL of methanol, 1.2 mL of methanol containing 13.7 μL of concentrated phosphoric acid was added, stirring was performed, standing was performed at −20° C. for 7 days, filtering was performed, a filter cake was dried in an oven at 50° C. for XRPD, DSC, TGA and DVS analysis, the X-ray powder diffraction pattern was shown in FIG. 5, and the DSC, TGA and DVS analysis were shown in FIG. 16-17.

### Example 10. Preparation of Mesylate Crystalline Form I

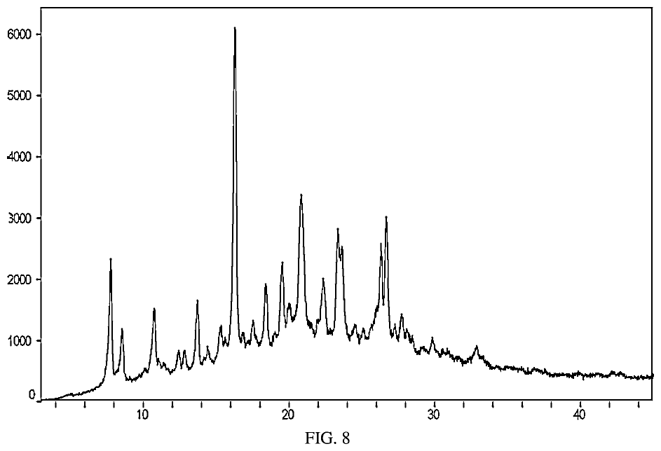

About 10 mg of the compound of the formula (I) was dissolved in 0.5 mL of tetrahydrofuran, 0.1 mL of tetrahydrofuran containing 2.29 μL of methanesulfonic acid was added, stirring was performed, standing was performed at −20° C. for 3 days, filtering was performed, a filter cake was dried in an oven at 50° C. for XRPD analysis, and the X-ray powder diffraction pattern was shown in FIG. 8.

### Example 11. Preparation of Mesylate Crystalline Form II

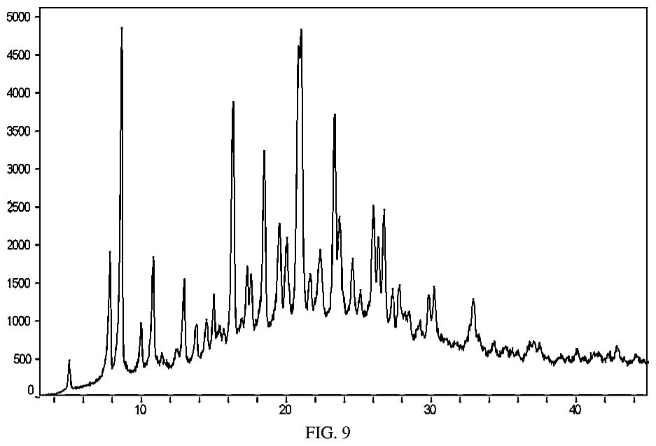

About 10 mg of the compound of the formula (I) was dissolved in 0.5 mL of acetone, 0.1 mL of acetone containing 2.29 μL of methanesulfonic acid was added, stirring was performed, standing was performed at −20° C. for 3 days, filtering was performed, a filter cake was dried in an oven at 50° C. for XRPD analysis, and the X-ray powder diffraction pattern was shown in FIG. 9.

### Example 12. Preparation of Mesylate Crystalline Form II

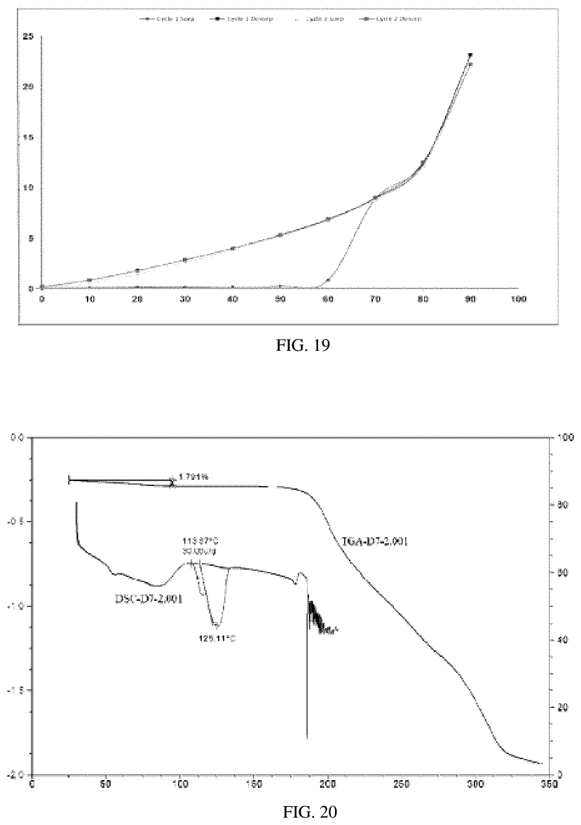

About 50 mg of the compound of the formula (I) was dissolved in 1.2 mL of acetone, 1.2 mL of acetone containing 11.4 μL of methanesulfonic acid was added, stirring was performed, standing was performed at −5° C. for 3 days, filtering was performed, a filter cake was dried in an oven at 50° C. for XRPD, DSC, TGA and DVS analysis, the X-ray powder diffraction pattern was shown in FIG. 9, and the DSC, TGA and DVS analysis were shown in FIG. 18-19.

### Example 13. Preparation of Citrate Crystalline Form I

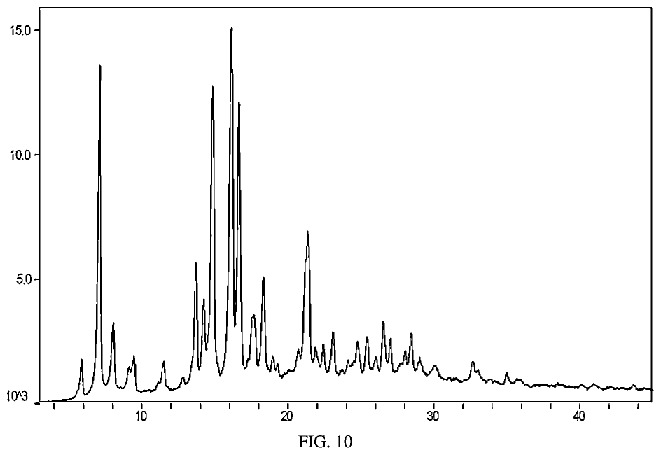

About 10 mg of the compound of the formula (I) was dissolved in 0.5 mL of acetonitrile, 4.57 mg of citric acid was added, stirring was performed at room temperature for 3 days, filtering was performed, a filter cake was dried in an oven at 50° C. for XRPD analysis, and the X-ray powder diffraction pattern was shown in FIG. 10.

### Example 14. Preparation of Malate Crystalline Form I

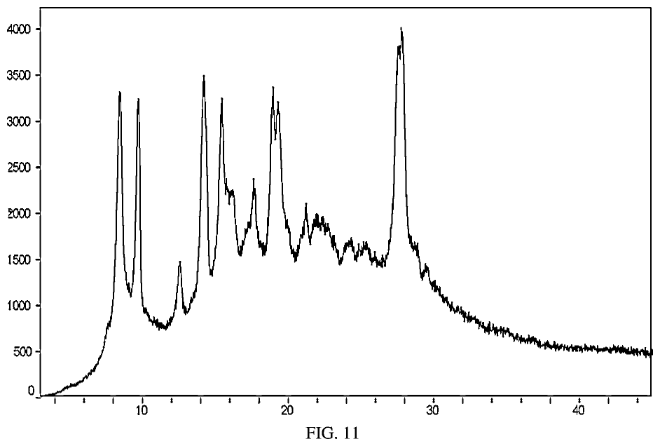

About 10 mg of the compound of the formula (I) was dissolved in 0.5 mL of ethyl acetate, 2.76 mg of malic acid was added, stirring was performed at room temperature for 3 days, filtering was performed, a filter cake was dried in an oven at 50° C. for XRPD analysis, and the X-ray powder diffraction pattern was shown in FIG. 11.

### Example 15. Preparation of Tartrate Crystalline Form I

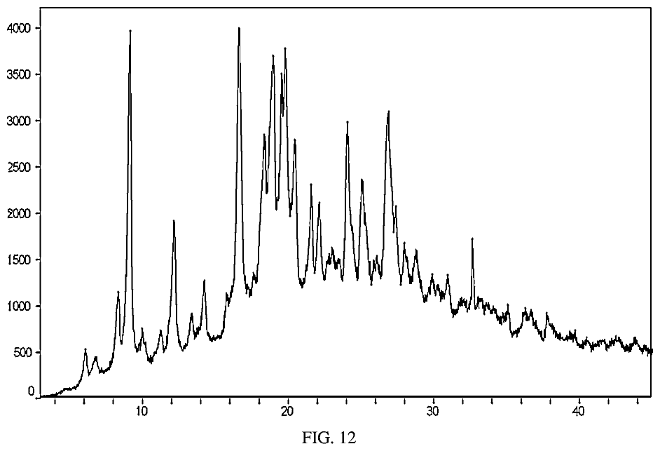

About 10 mg of the compound of the formula (I) was dissolved in 0.5 mL of acetonitrile, 3.57 mg of tartaric acid was added, stirring was performed, a solvent was volatilized, a solid was dried in an oven at 50° C. for XRPD analysis, and the X-ray powder diffraction pattern was shown in FIG. 12.

### Example 16. Preparation of Tartrate Crystalline Form I

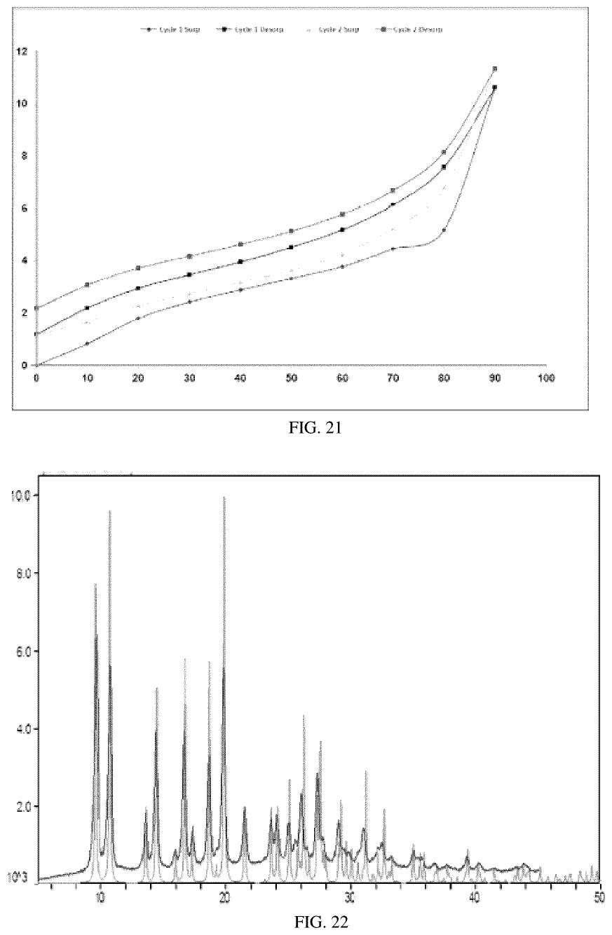

About 50 mg of the compound of the formula (I) was dissolved in 1.2 mL of acetonitrile, 17.87 mg of tartaric acid, 1 mL of acetonitrile and 0.2 mL of purified water were added, stirring was performed without precipitation, standing was performed at 5° C. for one week without precipitation, standing was performed at 5° C. for one week without precipitation, 1 mL of methyl tertiary butyl ether was added, stirring was performed, standing was performed at −20° C. for one week, filtering was performed, a filter cake was dried in an oven at 50° C. for XRPD, DSC, TGA and DVS analysis, the X-ray powder diffraction pattern was shown in FIG. 12, and the DSC, TGA and DVS analysis were shown in FIG. 20-21.

### Example 17. Preparation of Fumarate Crystalline Form I

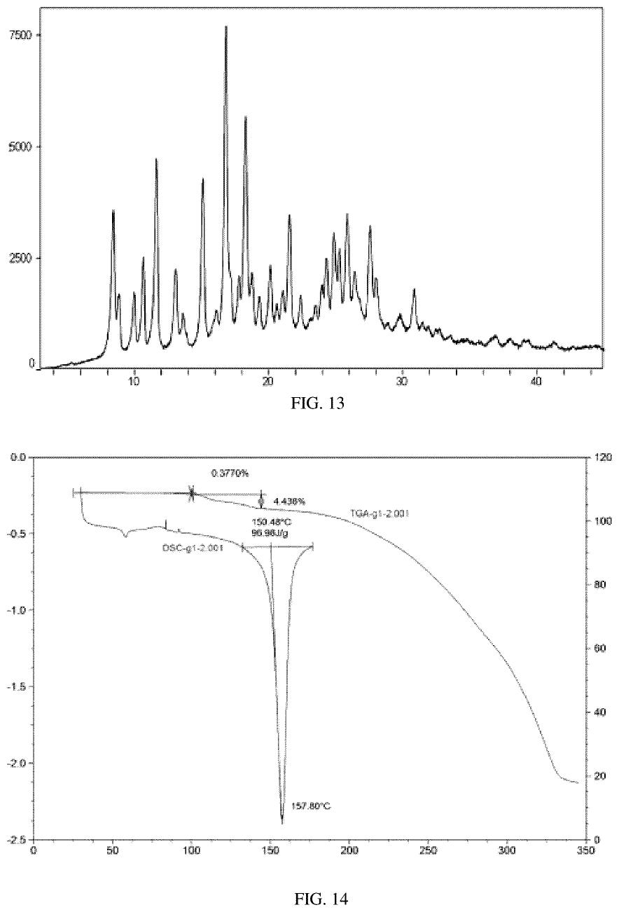

About 10 mg of the compound of the formula (I) was dissolved in 0.5 mL of methyl tertiary butyl ether, 2.76 mg of fumaric acid was added, stirring was performed, standing was performed at −20° C. for 3 days, filtering was performed, a filter cake was dried in an oven at 50° C. for XRPD analysis, and the X-ray powder diffraction pattern was shown in FIG. 13.

### Example 18. Structural Analysis of Hydrochloride Crystalline Form I

40 mg of the compound of the formula (I) was weighed and put in a 20 mL vial, 3 mL of acetone was added to dissolve the compound, and then 40 μL OF CONCENTRATED HYDROCHLORIC ACID (12M) WAS ADDED TO OBTAIN A SUSPENSION. Filtering was performed by using an FPTE syringe filter with a hole diameter of 0.22 mum to obtain a settled solution. 0.2 mL of filtrate was put in a 2 mL vial, the vial was sealed with a sealing film, small holes were made in the sealing film, and slow volatilization was performed at room temperature to obtain a small-sized platy monocrystal as a seed crystal.

40 mg of the compound of the formula (I) was weighed and put in a 20 mL vial, 3 mL of acetone was added to dissolve the compound, and then 40 μL of concentrated hydrochloric acid (12M) was added to obtain a suspension. Filtering was performed by using an FPTE syringe filter with a hole diameter of 0.22 mum to obtain a settled solution. 0.4 mL of filtrate was put in a 2 mL vial, then 0.3 mL of acetone was added, a small amount of the prepared platy seed crystal was added, the vial was sealed with a sealing film, small holes were made in the sealing film, and slow volatilization was performed at room temperature to obtain a small-sized platy monocrystal.

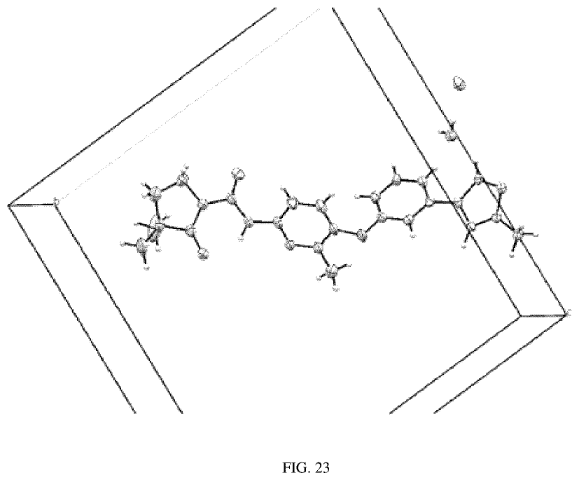

The appropriate monocrystal was selected to be detected on a Bruker APEX-II CCD single-crystal diffractometer. The temperature was maintained at 220 K during data collection. A single-crystal diffraction pattern was simulated as a powder crystal diffraction pattern through calculation with Mercury 3.10.2 (Build 189770) software and compared with the powder crystal diffraction pattern of the hydrochloride crystalline form I of the compound of the formula (I). As shown in FIG. 22, through comparison, it can be seen that the hydrochloride crystalline form I was consistent with the single-crystal simulated diagram and no excessive peak was generated, which indicated that the hydrochloride crystalline form I of the compound of the formula (I) in the present invention is a pure phase. Meanwhile, it can also be confirmed that the hydrochloride crystalline form I of the compound of the formula (I) is monohydrochloride monohydrate. The single-crystal cell structure was shown in FIG. 23.

| | |
|---|---|
| Molecular formula: | Z = 4 |
| C₂₂H₂₇CIN₆O₄ | |
| Mᵣ = 474.94 | F(000) = 1000.0 |
| Space group, Pca21 | Density Dₓ = 1.338 Mg m⁻³ |
| a = 6.910 (3) Å | Mo K radiation, = 0.71073 Å |
| b = 18.483 (6) Å | Linear absorption coefficient = 0.203 |
| | mm⁻¹ |
| c = 18.463 (7) Å | Test temperature T = 220 K |
| Cell volume V = 2358.0(15) Å³ | Single-crystal size: 0.22*0.2*0.05 mm |

### Example 19 Solubility Determination

About 2 mg of various crystalline acid salts and the free compound of the formula (I) were weighed respectively and put in a 2 mL glass vial, precise weighing was performed, about 100 μL of deionized water was added each time, ultrasonic treatment was performed, if the compound was not dissolved, deionized water was continued to add, ultrasonic treatment was performed till the compound was completely dissolved or the concentration was less than 0.2 mg/mL, the total volume of added water was recorded, the dissolution behavior and phenomenon were observed, and the solubility of each compound was calculated. The solubility test results were shown in the following table:

| | Preparation | | Dissolution |
|---|---|---|---|
| Crystalline Form | Method | Solubility S | Phenomenon |
| Hydrochloride crystalline | Example 4 | S > 20 mg/mL | Settled solution |
| form I | | | |
| Sulfate crystalline form I | Example 5 | S > 20 mg/mL | Settled solution |
| Phosphate crystalline | Example 9 | S > 20 mg/mL | Settled solution |
| form I | | | |
| Mesylate crystalline | Example 12 | S > 20 mg/mL | Settled solution |
| form II | | | |
| Citrate crystalline form I | Example 13 | S > 20 mg/mL | Settled solution |
| Malate crystalline form I | Example 14 | S > 20 mg/mL | Settled solution |
| Tartrate crystalline | Example 16 | S > 20 mg/mL | Settled solution |
| form I | | | |
| Fumarate crystalline | Example 17 | S > 20 mg/mL | Settled solution |
| form I | | | |
| Free compound | API raw | S < 1.5 mg/mL | Non-settling |
| | materials | | |

It can be seen from the above experimental results that after the free compound of the formula (I) is salted to obtain the crystalline compound, the solubility of all the salts in water is greatly improved, which can meet the development need of drug clinical preparations. Therefore, the solubility and drug release behavior of the free compound of the formula (I) can be significantly improved after the free compound is salted to obtain the crystalline compound.

### Example 20 Hygroscopic Behavior Test

The inventors of the present invention measured the hygroscopic weight gain results (hygroscopic weight gain/pre-hygroscopic weight*100%) of various crystalline forms at each relative humidity by using the dynamic vapor sorption method, the hygroscopicity of different crystalline compounds was evaluated, and the results obtained were as follows:

| | Weight | | | | | | | | | |
|---|---|---|---|---|---|---|---|---|---|---|
| | Relative humidity % | | | | | | | | | |
| Each crystal | 0.0 | 10.0 | 20.0 | 30.0 | 40.0 | 50.0 | 60.0 | 70.0 | 80.0 | 90.0 |
| Hydrochloride | 0.00 | 0.13 | 0.24 | 0.34 | 0.42 | 0.52 | 0.63 | 0.81 | 1.37 | 19.40 |
| crystalline form I | | | | | | | | | | |
| Phosphate crystalline | 0.02 | 1.19 | 1.86 | 2.32 | 2.78 | 3.37 | 3.97 | 4.66 | 5.46 | 6.383 |
| form I | | | | | | | | | | |
| Mesylate crystalline | 0.00 | 0.11 | 0.18 | 0.20 | 0.24 | 0.31 | 0.88 | 8.93 | 12.44 | 23.15 |
| form II | | | | | | | | | | |
| Tartrate crystalline | 0.00 | 0.83 | 1.79 | 2.42 | 2.88 | 3.32 | 3.77 | 4.44 | 5.17 | 10.61 |
| form I | | | | | | | | | | |

The above experimental results showed that when the relative humidity of the hydrochloride crystalline form I was less than 80%, the hygroscopic weight gain of the crystal was less than 1%, only when the relative humidity was increased to 80%, the hygroscopicity of the crystal began to slowly increase, and when the relative humidity was increased to 90%, the hygroscopicity of the crystal was increased significantly. The hygroscopic property of the hydrochloride crystalline form I preferably met the requirement of clinical preparation storage, and also met the requirement of a large amount of hygroscopicity in a high-humid environment to accelerate crystal dissolution, which was favorable for the clinical preparation granulation process.

The mesylate crystalline form II also has the similar hygroscopic property as the hydrochloride crystalline form I, and the difference only lies in that the hygroscopicity of the crystal of the mesylate crystalline form II is significantly increased when the relative humidity is increased to 70%. The hygroscopic property also preferably meets the requirements of the clinical preparation granulation process.

The hygroscopic property of the tartrate crystalline form I is that with the increase of relative humidity, the hygroscopic weight gain of the crystal will be increased equally, and a good linear relationship is formed. The hygroscopic property is favorable for developing special preparations in subsequent studies by the inventors.

The hygroscopicity of the phosphate crystalline form I is also characterized by the fact that the crystal of the crystalline form remains less hygroscopicity regardless of the ambient relative humidity, which can be used for storage, transportation and production in different areas and facilitates the preparation of special preparations.

It is well known that the drug hygroscopicity is an important physicochemical property that affects the production, storage and content of drugs. Different drug preparations can be developed according to different hygroscopic characteristics to meet different clinical development needs of drugs. The physical form with low hygroscopicity makes the drugs more stable in production, storage and content. Therefore, the above crystalline compounds have good hygroscopic properties, especially the hydrochloride crystalline form I and the phosphate crystalline form I, which have more advantages in terms of developability compared with other crystalline salts.

At least the crystalline forms of Examples 1-17 demonstrate the means for achieving solid-state forms of the compound of Formula (I). At least the crystalline forms of Examples 1-17 demonstrate the means for achieving pharmaceutically acceptable salt forms of the compound of Formula (I). At least the crystalline forms of Examples 1, 6, 11, and 16 demonstrate the means for achieving such hygroscopicity profiles.

### Example 21. XRPD Characterization of Solid Salt Forms of the Compound of Formula (I)

As a further preferred solution, the acid salt of the crystalline compound of formula (I) is hydrochloride, and the X-ray powder diffraction pattern (XRPD) includes four or more peaks at diffraction angles (2θ) of 9.52±0.2°, 19.72±0.2°, 10.64±0.2°, 14.32±0.2°, 16.56±0.2°, 18.52±0.2° and 27.20±0.2°.

As the most preferred solution, the acid salt of the crystalline compound of formula (I) is the hydrochloride, the X-ray powder diffraction pattern is substantially the same as the peak at the diffraction angle (2θ) shown in FIG. 1 (±0.2°), and the X-ray powder diffraction data are shown in Table 1:

| 2θ (°) | Intensity % | 2θ (°) | Intensity % |
|---|---|---|---|
| 9.52 | 100 | 27.20 | 39 |
| 19.72 | 77.4 | 25.88 | 29.1 |
| 10.64 | 50.3 | 21.40 | 25.5 |
| 14.32 | 50 | 24.00 | 19.5 |
| 16.56 | 43.2 | 23.54 | 19.3 |
| 18.52 | 39 | 28.92 | 18.8 |
| 30.92 | 16.6 | 24.92 | 15.1 |
| 13.50 | 15.9 | 27.66 | 10 |
| 17.22 | 15.2 | 32.40 | 9.8 |

The crystalline hydrochloride is designated as the hydrochloride crystalline form I, and the melting point is 157.8° C.

As a further preferred solution, the acid salt of the crystalline compound of the formula (I) is the hydrochloride, and the X-ray powder diffraction pattern (XRPD) includes four or more peaks at diffraction angles (2θ) of 24.32±0.2°, 17.78±0.2°, 24.58±0.2°, 19.96±0.2°, 10.18±0.2, 21.34±0.2°, 18.06±0.2°, 28.10±0.2° and 18.42±0.2°.

As the most preferred solution, the acid salt of the crystalline compound of formula (I) is the hydrochloride, the X-ray powder diffraction pattern is substantially the same as the peak at the diffraction angle (2θ) shown in FIG. 2 (±0.2°), and the X-ray powder diffraction data are shown in Table 2:

| 2θ (°) | Intensity % | 2θ (°) | Intensity % |
|---|---|---|---|
| 24.32 | 100 | 18.06 | 44.9 |
| 17.78 | 76 | 28.10 | 41.3 |
| 24.58 | 75.6 | 18.42 | 40.9 |
| 19.96 | 49.7 | 25.26 | 31.8 |
| 10.18 | 47.9 | 8.26 | 31 |
| 21.34 | 47.2 | 28.58 | 26.8 |
| 13.92 | 19 | 15.88 | 13.8 |
| 16.50 | 19 | 11.90 | 13.1 |
| 14.92 | 14 | 20.38 | 12.6 |

The crystalline hydrochloride is designated as the hydrochloride crystalline form II, and the melting point is 120.6° C.

As a further preferred solution, the acid salt of the crystalline compound of formula (I) is the hydrochloride, and the X-ray powder diffraction pattern (XRPD) includes four or more peaks at diffraction angles (2θ) of 18.74±0.2°, 22.94±0.2°, 17.64±0.2°, 9.38±0.24, 9.10±0.2°, 9.94±0.2°, 29.70±0.2° and 11.24±0.2°.

As the most preferred solution, the acid salt of the crystalline compound of formula (I) is the hydrochloride, the X-ray powder diffraction pattern is substantially the same as the peak at the diffraction angle (2θ) shown in FIG. 3 (±0.2°), and the X-ray powder diffraction data are shown in Table 3:

| 2θ (°) | Intensity % | 2θ (°) | Intensity % |
|---|---|---|---|
| 18.74 | 100 | 29.70 | 31.4 |
| 22.94 | 87.2 | 11.24 | 30.7 |
| 17.64 | 72.5 | 8.78 | 23.8 |
| 9.38 | 71 | 27.00 | 22.2 |
| 9.10 | 62 | 26.54 | 18.5 |
| 9.94 | 35.3 | 35.76 | 13.4 |
| 28.28 | 12.5 | 34.34 | 11.3 |
| 33.20 | 11.8 | 30.12 | 11 |
| 14.66 | 11.7 | 21.16 | 10.4 |

The crystalline hydrochloride is designated as the hydrochloride crystalline form III, and the melting point is 110.9° C.

As a further preferred solution, the acid salt of the crystalline compound of formula (I) is sulfate, and the X-ray powder diffraction pattern (XRPD) includes four or more peaks at diffraction angles (2θ) of 20.08±0.2°, 23.22±0.2°, 21.38±0.2°, 24.86±0.2°, 18.78±0.2°, 20.46±0.2° and 9.38±0.2°.

As the most preferred solution, the acid salt of the crystalline compound of formula (I) is the sulfate, the X-ray powder diffraction pattern is substantially the same as the peak at the diffraction angle (2θ) shown in FIG. 4 (0.2°), and the X-ray powder diffraction data are shown in Table 4:

| 2θ (°) | Intensity % | 2θ (°) | Intensity % |
|---|---|---|---|
| 20.08 | 100 | 9.38 | 53.8 |
| 23.22 | 86.6 | 26.88 | 44 |
| 21.38 | 77 | 17.12 | 43.5 |
| 24.86 | 70.9 | 29.30 | 27.1 |
| 18.78 | 60.2 | 16.64 | 24.3 |
| 20.46 | 56.9 | 18.12 | 20.5 |
| 24.06 | 20 | 8.78 | 18 |
| 24.48 | 19.3 | 19.34 | 18 |
| 28.14 | 18.8 | 13.97 | 17.5 |

The crystalline sulfate is designated as sulfate crystalline form I.

As a further preferred solution, the acid salt of the crystalline compound of formula (I) is phosphate, and the X-ray powder diffraction pattern (XRPD) includes four or more peaks at diffraction angles (2θ) of 8.44±0.2°, 16.82±0.2°, 10.78±0.2, 18.10±0.2°, 24.78±0.2, 19.62±0.2° and 23.24±0.2°.

As the most preferred solution, the acid salt of the crystalline compound of formula (I) is the phosphate, the X-ray powder diffraction pattern is substantially the same as the peak at the diffraction angle (2θ) shown in FIG. 5 (0.2°), and the X-ray powder diffraction data are shown in Table 5:

| 2θ (°) | Intensity % | 2θ (°) | Intensity % |
|---|---|---|---|
| 8.44 | 100 | 23.24 | 22.1 |
| 16.82 | 66.2 | 21.36 | 19.9 |
| 10.78 | 53.2 | 27.44 | 17.7 |
| 18.10 | 41.8 | 15.90 | 17.2 |
| 24.78 | 24.6 | 23.58 | 15.6 |
| 19.62 | 22.1 | 11.68 | 15.4 |
| 21.76 | 15.1 | 26.12 | 12.5 |
| 27.92 | 14.3 | 20.36 | 10.8 |
| 25.42 | 13.7 | 15.26 | 9.4 |

The crystalline phosphate is designated as the phosphate crystalline form I, and the melting point is 154.2° C.

As a further preferred solution, the acid salt of the crystalline compound of formula (I) is the phosphate, and the X-ray powder diffraction pattern (XRPD) includes four or more peaks at diffraction angles (2θ) of 10.86±0.2°, 8.48±0.2°, 17.02±0.2°, 10.46±0.2°, 18.38±0.2°, 7.98±0.2°, 23.82±0.2° and 16.06±0.2°.

As the most preferred solution, the acid salt of the crystalline compound of formula (I) is the phosphate, the X-ray powder diffraction pattern is substantially the same as the peak at the diffraction angle (2θ) shown in FIG. 6 (±0.2°), and the X-ray powder diffraction data are shown in Table 6:

| 2θ (°) | Intensity % | 2θ (°) | Intensity % |
|---|---|---|---|
| 10.86 | 100 | 23.82 | 33.3 |
| 8.48 | 90.6 | 16.06 | 32.7 |
| 17.02 | 90.2 | 22.34 | 31.8 |
| 10.46 | 42.3 | 11.76 | 30.9 |
| 18.38 | 36.9 | 13.82 | 30.9 |
| 7.98 | 35.5 | 27.96 | 28.8 |
| 12.10 | 26.5 | 25.02 | 21.7 |
| 21.54 | 25.2 | 19.94 | 21.3 |
| 19.06 | 22.1 | 17.52 | 20.2 |

This crystalline phosphate is designated as the phosphate crystalline form II, and the melting point is 153.8° C.

As a further preferred solution, the acid salt of the crystalline compound of formula (I) is the phosphate, and the X-ray powder diffraction pattern (XRPD) includes four or more peaks at diffraction angles (2θ) of 10.84±0.2°, 8.54±0.2°, 17.14±0.2°, 16.76±0.2°, 10.36±0.2°, 18.26±0.2, 27.88±0.2° and 22.34±0.2°.

As the most preferred solution, the acid salt of the crystalline compound of formula (I) is the phosphate, the X-ray powder diffraction pattern is substantially the same as the peak at the diffraction angle (2θ) shown in FIG. 7 (0.2°), and the X-ray powder diffraction data are shown in Table 7:

| 2θ (°) | Intensity % | 2θ (°) | Intensity % |
|---|---|---|---|
| 10.84 | 100 | 27.88 | 22.2 |
| 8.54 | 44.4 | 22.34 | 20.3 |
| 17.14 | 32.8 | 25.06 | 17.6 |
| 16.76 | 27.4 | 23.70 | 17.1 |
| 10.36 | 24.7 | 13.78 | 16.2 |
| 18.26 | 23.9 | 11.68 | 16.1 |
| 16.04 | 13.1 | 26.14 | 9.5 |
| 20.10 | 12.7 | 21.08 | 9.1 |
| 19.34 | 11.8 | 12.08 | 8.7 |

The crystalline phosphate is designated as the phosphate crystalline form III, and the melting point is 147.3° C.

As a further preferred solution, the acid salt of the crystalline compound of formula (I) is mesylate, and the X-ray powder diffraction pattern (XRPD) includes four or more peaks at diffraction angles (2θ) of 16.28±0.2°, 20.82±0.2°, 7.78±0.2°, 26.68±0.2°, 23.36±0.2°, 26.30±0.2° and 23.62±0.2°.

As the most preferred solution, the acid salt of the crystalline compound of formula (I) is the mesylate, the X-ray powder diffraction pattern is substantially the same as the peak at the diffraction angle (2θ) shown in FIG. 8 (0.2°), and the X-ray powder diffraction data are shown in Table 8:

| 2θ (°) | Intensity % | 2θ (°) | Intensity % |
|---|---|---|---|
| 16.28 | 100 | 23.62 | 27.8 |
| 20.82 | 42 | 10.74 | 20.7 |
| 7.78 | 39.2 | 19.54 | 20.4 |
| 26.68 | 36.5 | 13.72 | 20.2 |
| 23.36 | 33.2 | 18.40 | 18.5 |
| 26.30 | 29.3 | 22.34 | 17.2 |
| 8.54 | 14.8 | 15.34 | 8.5 |
| 25.96 | 9 | 17.54 | 7.4 |
| 27.72 | 8.6 | 12.44 | 6.3 |

The crystalline mesylate is designated as the mesylate crystalline form I, and the melting point is 184.4° C.

As a further preferred solution, the acid salt of the crystalline compound of formula (I) is the mesylate, and the X-ray powder diffraction pattern (XRPD) includes four or more peaks at diffraction angles (2θ) of 8.64±0.2°, 21.02±0.2°, 16.34±0.2°, 23.34±0.2°, 18.48±0.2°, 7.84±2°, 26.00±0.2° and 10.82±0.2°.

As the most preferred solution, the acid salt of the crystalline compound of formula (I) is the mesylate, the X-ray powder diffraction pattern is substantially the same as the peak at the diffraction angle (2θ) shown in FIG. 9 (10.20), and the X-ray powder diffraction data are shown in Table 9:

| 2θ (°) | Intensity % | 2θ (°) | Intensity % |
|---|---|---|---|
| 8.64 | 100 | 10.82 | 32 |
| 21.02 | 82.6 | 26.74 | 30.6 |
| 16.34 | 68.4 | 23.66 | 28.5 |
| 23.34 | 58.8 | 19.52 | 27.8 |
| 18.48 | 51.4 | 12.96 | 24.6 |
| 7.84 | 38.3 | 26.34 | 23.8 |
| 26.00 | 32.7 | 20.04 | 22.1 |
| 17.32 | 18.2 | 17.56 | 15.8 |
| 22.34 | 18.1 | 15.00 | 15.6 |
| 24.58 | 16.3 | 30.20 | 15.3 |

This crystalline mesylate is designated as the mesylate crystalline form II, and the melting point is 185.5° C.

As a further preferred solution, the acid salt of the crystalline compound of formula (I) is citrate, and the X-ray powder diffraction pattern (XRPD) includes four or more peaks at diffraction angles (2θ) of 16.14±0.2°, 7.12±0.2°, 14.86±0.2°, 16.64±0.2°, 21.34±0.2° and 13.70±0.2°.

As the most preferred solution, the acid salt of the crystalline compound of formula (I) is the citrate, the X-ray powder diffraction pattern is substantially the same as the peak at the diffraction angle (2θ) shown in FIG. 10 (±0.2°), and the X-ray powder diffraction data are shown in Table 10:

| 2θ (°) | Intensity % | 2θ (°) | Intensity % |
|---|---|---|---|
| 16.14 | 100 | 13.70 | 31.7 |
| 7.12 | 96.1 | 18.34 | 26.2 |
| 14.86 | 83 | 14.24 | 22 |
| 16.64 | 76.9 | 8.04 | 18.7 |
| 21.34 | 39.8 | 17.64 | 15.4 |
| 26.50 | 14 | 28.44 | 11.4 |
| 5.88 | 11.8 | 9.44 | 10.1 |
| 23.08 | 11.6 | 25.38 | 9.8 |

The crystalline citrate is designated as the citrate crystalline form I, and the melting point is 58.1° C.

As a further preferred solution, the acid salt of the crystalline compound of formula (I) is malate, and the X-ray powder diffraction pattern (XRPD) includes four or more peaks at diffraction angles (2θ) of 8.44±0.2°, 27.82±0.2°, 14.22±0.2°, 9.72±0.2°, 15.44±0.2°, 18.96±0.2° and 19.28±0.2°.

As the most preferred solution, the acid salt of the crystalline compound of formula (I) is the malate, the X-ray powder diffraction pattern is substantially the same as the peak at the diffraction angle (2θ) shown in FIG. 11 (10.20), and the X-ray powder diffraction data are shown in Table 11:

| 2θ (°) | Intensity % | 2θ (°) | Intensity % |
|---|---|---|---|
| 8.44 | 100 | 19.28 | 67.1 |
| 27.82 | 99.8 | 17.64 | 31.9 |
| 14.22 | 95.1 | 12.58 | 23.9 |
| 9.72 | 93.2 | 7.61 | 21.4 |
| 15.44 | 72.4 | 16.20 | 21 |
| 18.96 | 69.1 | 21.22 | 18.5 |
| 22.38 | 13.3 | 24.32 | 9 |
| 17.24 | 12.3 | 25.38 | 7.7 |
| 21.98 | 11.7 | 20.88 | 6.5 |

The crystalline malate salt is designated as the malate crystalline form I, and the melting point is 82.8° C.

As a further preferred solution, the acid salt of the crystalline compound of formula (I) is tartrate, and the X-ray powder diffraction pattern (XRPD) includes four or more peaks at diffraction angles (2θ) of 9.16±0.2°, 16.64±0.2°, 19.80±0.2°, 26.84±0.2°, 18.96±0.2°, 24.06±0.2° and 12.16±0.2°.

As the most preferred solution, the acid salt of the crystalline compound of formula (I) is the tartrate, the X-ray powder diffraction pattern is substantially the same as the peak at the diffraction angle (2θ) shown in FIG. 12 (±0.2°), and the X-ray powder diffraction data are shown in Table 12:

| 2θ (°) | Intensity % | 2θ (°) | Intensity % |
|---|---|---|---|
| 9.16 | 100 | 12.16 | 40.8 |
| 16.64 | 86.7 | 18.36 | 34.3 |
| 19.80 | 58.1 | 20.44 | 31 |
| 26.84 | 51.2 | 21.56 | 30.8 |
| 18.96 | 50.1 | 25.02 | 30.7 |
| 24.06 | 48.4 | 8.34 | 25.4 |
| 22.14 | 24.1 | 14.26 | 18 |
| 27.40 | 22.3 | 25.32 | 17.2 |
| 32.66 | 21.8 | 24.40 | 15.3 |

The crystalline tartrate is designated as the tartrate crystalline form I, and the melting point is 122.4° C.

As a further preferred solution, the acid salt of the crystalline compound of formula (I) is fumarate, and the X-ray powder diffraction pattern (XRPD) includes four or more peaks at diffraction angles (2θ) of 16.82±0.2°, 18.28±0.2°, 11.62±0.2°, 15.10±0.2°, 8.44±0.2°, 21.54±0.2° and 27.58±0.2°.

As the most preferred solution, the acid salt of the crystalline compound of formula (I) is the fumarate, the X-ray powder diffraction pattern is substantially the same as the peak at the diffraction angle (2θ) shown in FIG. 13 (±0.2°), and the X-ray powder diffraction data are shown in Table 13:

| 2θ (°) | Intensity % | 2θ (°) | Intensity % |
|---|---|---|---|
| 16.82 | 100 | 27.58 | 31.8 |
| 18.28 | 69.3 | 25.84 | 29.1 |
| 11.62 | 62.8 | 10.68 | 28.5 |
| 15.10 | 53.5 | 13.06 | 25.8 |
| 8.44 | 48.1 | 24.86 | 23.5 |
| 21.54 | 36.1 | 20.12 | 19 |
| 8.86 | 18.5 | 24.32 | 16.8 |
| 9.98 | 18.3 | 17.16 | 15.7 |
| 25.28 | 17.3 | 27.98 | 15 |

This crystalline fumarate is designated as the fumarate crystalline form I.

### Example 22. Formulations of Crystalline Salts of the Compound of Formula (I)

Formulations containing the crystalline salt forms of Examples 1-17 are prepared. Such formulations contain the crystalline salt forms in an amount sufficient to provide 25 or 50 mg of the compound of Formula (I).

### Example 23. A Clinical Study of the Treatment of TGCT in Patients Using Capsules of the Compound of Formula (I)

The goal of this clinical trial is to assess the efficacy and safety of the compound of Formula (I) in patients with Tenosynovial Giant Cell Tumor (TGCT). The main questions it aims to answer are: whether the compound of Formula (I) works well in patients with TGCT and whether the compound of Formula (I) is safe in patients with TGCT. During the study, patients receive a 50 mg or 75 mg once daily dose of the compound of Formula (I).

This study consists of part 1 and part 2. Part 1 is a double-blind phase, eligible patients will be randomized to treatment group to receive capsule formulations containing 25 mg or 50 mg of the Compound of Formula (I) for 24 weeks or matching placebo group for 24 weeks. Capsules may provide a pharmaceutically acceptable salt of the compound of Formula (I) or hydrate thereof, such as a hydrochloride monohydrate of the compound of Formula (I), in an amount sufficient to provide 25 mg or 50 mg of the compound of Formula (I).

Part 2 is an open-label treatment phase, and all patients entering this phase will receive open-label compound of Formula (I) after 24 weeks of dosing in the blinded study of Part 1 or withdrawal from the study.

### EQUIVALENTS

Those skilled in the art will recognize or be able to ascertain using no more than routine experimentation, numerous equivalents to the specific embodiments described specifically in this disclosure. Such equivalents are intended to be encompassed in the scope of the following claims.

1. A pharmaceutically acceptable formulation comprising a compound of Formula (I): 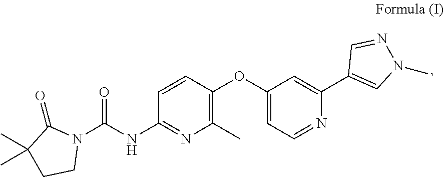
and one or more pharmaceutically acceptable excipients, wherein the compound is present in the pharmaceutically acceptable formulation as a free base, or a pharmaceutically acceptable salt of the compound and wherein the free base or pharmaceutically acceptable salt is in anhydrous or hydrate form.

2. The pharmaceutically acceptable formulation of claim 1, wherein the compound of Formula (I) is present in the pharmaceutically acceptable formulation as a monohydrate or dihydrate of a pharmaceutically acceptable salt.

3. The pharmaceutically acceptable formulation of claim 1, wherein the compound of Formula (I) is present in the pharmaceutically acceptable formulation as an anhydrous form or a monohydrate of a hydrochloride salt.

4. The pharmaceutically acceptable formulation of claim 1, wherein the one or more pharmaceutically acceptable excipients is a filler.

5. The pharmaceutically acceptable formulation of claim 1, wherein the one or more pharmaceutically acceptable excipients is lactose or lactose monohydrate.

6. The pharmaceutically acceptable formulation of claim 1, further comprising one or more disintegrants, one or more lubricants, or a combination thereof.

7. The pharmaceutically acceptable formulation of claim 1, further comprising one or more disintegrants.

8. The pharmaceutically acceptable formulation of claim 1, further comprising one or more lubricants.

9. A pharmaceutically acceptable unit formulation comprising:
(a) about 1% by weight to about 70% by weight, based on the weight of the pharmaceutically acceptable unit formulation, of a compound of Formula (I): 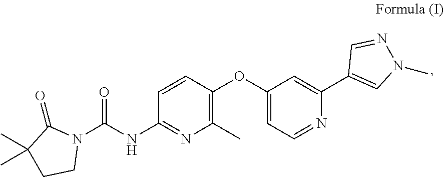
wherein the compound is present in the pharmaceutically acceptable unit formulation as a free base, or a pharmaceutically acceptable salt of the compound and wherein the free base or pharmaceutically acceptable salt is in anhydrous or hydrate form, and one or more pharmaceutically acceptable excipients, wherein the one or more pharmaceutically acceptable excipients comprise:
(b) about 20% by weight to about 97% by weight of one or more fillers based on the weight of the pharmaceutically acceptable unit formulation;
(c) about 1% by weight to about 20% by weight of one or more disintegrants based on the weight of the pharmaceutically acceptable unit formulation; and
(d) about 0.1% by weight to about 10% by weight of one or more lubricants based on the weight of the pharmaceutically acceptable unit formulation.

10. The pharmaceutically acceptable unit formulation of claim 9, comprising:
(a) about 10% by weight to about 30% by weight of the compound of Formula (I) based on the weight of the pharmaceutically acceptable unit formulation;
(b) about 70% by weight to about 90% by weight of one or more fillers based on the weight of the pharmaceutically acceptable unit formulation;
(c) about 1% by weight to about 10% by weight of one or more disintegrants based on the weight of the pharmaceutically acceptable unit formulation; and
(d) about 0.1% by weight to about 3% by weight of one or more lubricants based on the weight of the pharmaceutically acceptable unit formulation.

11. The pharmaceutically acceptable unit formulation of claim 9, wherein the one or more fillers is selected from the group consisting of calcium phosphate, dicalcium phosphate, dicalcium phosphate dihydrate, calcium sulfate, calcium sulfate dihydrate, starch, calcium carbonate, magnesium carbonate, magnesium oxide, kaolin (natural hydrated aluminum silicate), sodium chloride, partially gelatinized starch, anhydrous lactose, lactose monohydrate, lactose dihydrate, trehalose dihydrate, spray dried lactose, sucrose, dextrose, dextrates, dextrin, cellulose, microcrystalline cellulose, silicified microcrystalline cellulose, powdered cellulose, partially depolymerized cellulose, mannitol, granulated mannitol, spray dried mannitol, maltodextrin, maltitol, confectioner's sugar, compressible sugar, sorbitol, starch and talc.

12. (canceled)

13. The pharmaceutically acceptable unit formulation of claim 9, wherein the one or more disintegrants is selected from the group consisting of sodium starch glycolate, pregelatinized starch, clay, cellulose, alginic acid, alginate gum, polyvinylpyrrolidone, cross-linked polyvinylpyrrolidone, cross-linked calcium carboxymethylcellulose, cross-linked sodium carboxymethylcellulose, sodium croscarmellose, sodium carmellose, low substituted hydroxypropyl cellulose, low substituted hydroxypropyl cellulose sodium, guar gum, magnesium aluminum silicate, polacrilin potassium, powdered cellulose, sodium alginate and soy polysaccharides.

14. (canceled)

15. The pharmaceutically acceptable unit formulation of claim 9, wherein the one or more lubricants is selected from the group consisting of colloidal silica, magnesium trisilicate, talc, magnesium carbonate, magnesium oxide, glyceryl behaptate, mono, di and tri glyceryl behenate, bees wax, behenoyl polyoxyl-8 glycerides, hydrogenated vegetable oil, polyethylene glycol, ethylene oxide polymer, sodium lauryl sulfate, magnesium stearate, aluminum stearate, calcium stearate, sodium stearyl fumarate, stearic acid, magnesium lauryl stearate, mixtures of magnesium stearate with sodium lauryl sulfate.

16. (canceled)

17. The pharmaceutically acceptable unit formulation of claim 9, wherein the one or more pharmaceutically acceptable excipients is lactose monohydrate and the pharmaceutically acceptable unit formulation further comprises cross-linked polyvinylpyrrolidone and magnesium stearate.

18. The pharmaceutically acceptable unit formulation of claim 12, wherein the pharmaceutically acceptable unit formulation further comprises cross-linked polyvinylpyrrolidone and magnesium stearate.

19. The pharmaceutically acceptable unit formulation of claim 9, wherein the pharmaceutically acceptable unit formulation is for oral administration.

20. The pharmaceutically acceptable unit formulation of claim 9, wherein the pharmaceutically acceptable unit formulation is a solid dosage form.

21. The pharmaceutically acceptable unit formulation of claim 9, wherein the pharmaceutically acceptable unit formulation is a tablet or a capsule.

22. (canceled)

23. The pharmaceutically acceptable unit formulation of claim 9, wherein the pharmaceutically acceptable unit formulation comprises about 1% by weight to about 30% by weight of the compound of Formula (I) based on the weight of the pharmaceutically acceptable unit formulation.

24-32. (canceled)

33. The pharmaceutically acceptable unit formulation of claim 9, having an acceptance value of less than about 15 as determined by United States Pharmacopeia (USP) Uniformity of Dosage Unit <905>.

34-245. (canceled)
